(19) United States
(12) Patent Application Publication
(10) Pub. No.: US 2012/0036593 A1
(43) Pub. Date: Feb. 9, 2012

(54) Plant Promoter Operable in Endosperm and Uses Thereof

(75) Inventors: German Spangenberg, Victoria (AU); Ulrik John, Westgarth (AU); Carl Ramage, Mitcham (AU); Huihua Fu, Cary, NC (US); Rui-Guang Zhen, Chapel Hill, NC (US); Hee-Sook Song, Raleigh, NC (US)

(73) Assignee: BASF Plant Science Company GmbH, Ludwigshafen (DE)

(21) Appl. No.: 13/264,559

(22) PCT Filed: Apr. 16, 2010
(86) PCT No.: PCT/AU2010/000430

§ 371 Date: Oct. 25, 2011

Related U.S. Application Data
(60) Provisional application No. 61/170,171, filed on Apr. 17, 2009.

(51) Int. Cl. C12N 15/82; A01H 5/00; C12N 15/64; C12N 5/10; C12N 15/113; C12N 15/63

(52) U.S. Cl. 800/278; 536/24.1; 435/320.1; 435/419; 800/298; 435/468

(57) ABSTRACT

The present invention provides compositions of matter comprising plant-operable promoter sequences that confer selective/specific endosperm expression on genes to which they are operably connected and uses of such compositions to confer gene expression, especially in developing endosperm.

### RELATED APPLICATIONS

The application claims the benefit of priority from U.S. patent application No. 61/170,171 filed Apr. 17, 2009, the content of which is incorporated herein in its entirety.

### FIELD OF THE INVENTION

The present invention relates to compositions of matter comprising plant-operable promoter sequences and regulatory sequences derived therefrom and to uses of such compositions to confer gene expression, especially in developing endosperm.

### BACKGROUND OF INVENTION

### Description of the Related Art

To date plants have been genetically modified for a variety of reasons, including to confer pest resistance, e.g., by expressing antifungal or antibacterial proteins, or improving an agronomic trait, e.g., by modulating fruit ripening, or inducing sterility in a hybrid plant or for the large-scale production of proteins for industrial, pharmaceutical, veterinary and agricultural use. In this respect, advances in biotechnological research have produced an explosion of information in relation to the number of nucleic acids identified which, if appropriately expressed, are useful to produce improved plants, for example, plants resistant to pre-harvest sprouting, plants having an improved nutritional quality, plants having a pharmaceutical quality, plants in which reproductive development is controlled, plants having altered shape or size characteristics, plants capable of rapid regeneration following harvest, or plants having improved resistance to pathogens, amongst others.

However, a problem associated with the genetic improvement of agriculturally-important plants, for example, crops, is the manipulation of gene expression to produce plants which exhibit novel characteristics. In this respect, it is often desirable that a nucleic acid to be expressed in a plant is preferentially or selectively expressed, or expressed specifically, in one or more specific cell types, tissues or organs of the plant, or under specific environmental or developmental conditions, rather than constitutively expressed.

Moreover, as more genes having desirable agronomic or pharmaceutical value become available, the need for transformed plants with multiple genes will increase exponentially. These multiple exogenous genes must typically be controlled by separate regulatory sequences, to provide appropriate levels and patterns of expression which may not be the same for each structural gene or other transgene to be expressed. For example, some genes may need to be expressed constitutively whereas other genes will need to be expressed at certain developmental stages or locations in the transgenic organism. Accordingly, a variety of regulatory sequences having diverse effects is needed.

By “preferentially” as used throughout the specification and claims is meant that a promoter confers expression on a nucleic acid to which it is operably linked to a greater extent or higher level in one or more specific cell types, tissues or organs of a plant, or under specific environmental or developmental conditions than it does in one or more other cells, tissues or organs or under another condition. However, the term “preferentially” does not limit the expression of the nucleic acid to the one or more specific cell types, tissues or organs of a plant, or under specific environmental or developmental conditions. Rather, the level of expression need only be increased to a higher level, and preferably significantly increased.

By “selectively” is meant that a promoter confers expression on a nucleic acid to which it is operably linked to in one or more specific cell types, tissues or organs of a plant, or under specific environmental or developmental conditions.

By “specifically” is meant exclusively.

As used throughout this specification and in the claims that follow, and unless the context requires otherwise, the word “confer” and variations thereof such as “conferring” shall be taken to mean the ability of a promoter or an active fragment or derivative thereof, for example in the context of other factors such as DNA conformation and/or cis-acting DNA sequence(s) and/or trans-acting factor(s) and/or signalling pathway(s) and/or transcript structure and/or transcript processing, to produce expression or a pattern of expression of nucleic acid to which the promoter or active fragment or derivative is operably-connected in response to one or more developmental and/or environmental and/or hormonal and/or other stimuli that would normally elicit the expression or pattern of expression for nucleic acid to which the promoter is operably-connected in its native context.

As used throughout this specification and in the claims that follow, the term “promoter” is to be taken in its broadest context and includes transcriptional regulatory sequences of a classical genomic gene, including a basal promoter regulatory region comprising a TATA box which is required for transcription initiation with or without a CCAAT box sequence, and optional additional regulatory elements (e.g., upstream activating sequences, enhancers and silencers) which alter gene expression in response to developmental and/or hormonal and/or environmental stimuli, or in a tissue-specific or cell-type-specific manner. A promoter is usually, but not necessarily, positioned upstream, or 5′, of a structural gene, upon which it confers expression. Furthermore, the regulatory elements comprising a promoter are usually positioned within 2 kb of the start site of transcription of a plant gene.

As used throughout this specification and in the claims that follow, and unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

As used throughout this specification and in the claims that follow, the term “active fragment” in the context of a promoter shall be taken to mean a fragment or region or portion of a promoter that retains the ability of the promoter from which it is derived to initiate transcription. Such an active fragment need not necessarily confer expression or a pattern of expression on a nucleic acid to which it is operably connected in the same manner as the promoter from which it is derived. For example, an active fragment of a promoter induces the level of expression of a nucleic acid to a higher or lower degree than a promoter from which it is derived. Alternatively, or in addition, an active fragment of a promoter confers expression in a different cell, tissue or organ, or in fewer tissues or in an additional cell, tissue or organ to that in which a promoter from which it is derived confers expression. Methods for identifying such an active fragment will be apparent to the skilled artisan and/or described herein.

As used throughout this specification and in the claims that follow, the term “derivative” in the context of a promoter shall be taken to mean a promoter derived from a promoter as described herein according to any embodiment, e.g., a promoter comprising one or more additional regulatory elements, e.g., to increase or reduce or otherwise control expression of a nucleic acid operably connected thereto. The present invention also encompasses a derivative comprising a promoter as described herein according to any embodiment linked to another promoter, e.g., a bi-directional promoter. In this respect, the other promoter may also be a promoter as described herein according to any embodiment. The term “derivative” also encompasses a promoter comprising a variation in its sequence relative to a promoter as described herein according to any embodiment. For example, the sequence of such a derivative may include one or more of the following variations: a deletion, an insertion, a single or multiple point mutation or an alteration at a particular restriction enzyme site, provided that the derivative promoter retains its ability to initiate and/or suppress transcription of a nucleic acid linked thereto.

As used throughout this specification and in the claims that follow, the term “expression” or similar term such as “express” shall be taken to refer de minimis to transcription of a nucleic acid to produce RNA and to optionally encompass such transcription and subsequent translation of transcribed RNA to produce a peptide, polypeptide or protein. This definition is not to be limited to any specific cellular context and includes e.g., such expression obtained using in vitro expression systems or in isolated cells, tissues or organs.

Similarly, a “pattern of expression” refers to one or more of the timing, level, cellular location, sub-cellular location, tissue-selectivity or organ-selectivity of expression as hereinbefore defined, including the relative expression in one cell, tissue or organ compared to another cell, tissue or organ, and including the relative level or relative timing of expression such as at different developmental stages or in response to different environmental or hormonal stimuli.

As used throughout this specification and in the claims that follow, the term “operable” will be understood to mean the ability of a stated integer to function in a particular context albeit not necessarily only in that stated context.

As used throughout this specification and in the claims that follow, the terms “operably connected” and “in operable connection with” mean the positioning of a promoter of the present invention or active fragment or derivative thereof in spatial relation to another nucleic acid, (e.g., a transgene including a structural gene, open reading frame, reporter gene, or nucleic acid encoding a ribozyme, minizyme, RNAi molecule or other RNA) to thereby confer expression on said other nucleic acid by the promoter, active fragment or derivative. Thus, the relative positioning of the promoter, active fragment or derivative to the other nucleic acid produces a structure that confer a functional expression pattern on the other nucleic acid. A promoter is generally positioned 5′ (upstream) to the nucleic acid, the expression of which it controls. To construct heterologous promoter/nucleic acid combinations (e.g., promoter/transgene and/or promoter/selectable marker gene combinations), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

As used throughout this specification and in the claims that follow, the term “native context” in the present context shall be taken to mean a genomic gene in which a promoter naturally occurs in the genome of a plant, i.e., from which the promoter is isolated. The genomic gene in which a promoter is located in nature may be identified and/or subjected to sequence comparison using sequence analysis software available from, for example National Center for Biotechnology Information (NCBI) at the National Library of Medicine at the National Institutes of Health of the Government of the United States of America, Bethesda, Md., 20894, United States of America.

In angiosperms, the seed endosperm forms a nutritive tissue for the embryo. For example, the endosperm of cereals originates with a series of free-nuclear divisions, followed by cellularisation and the subsequent formation of a range of functional cellular domains. This tissue is complex in its structure and development, particularly in cereals. The uptake of assimilates by the growing endosperm is a critical process in seed development. The central area of the endosperm consists of large vacuolated cells that store the reserves of starch and highly-abundant storage proteins.

The ability to express a recombinant nucleic acid in endosperm is desirable for the production of heterologous proteins, e.g., for pharmaceutical or industrial purposes. For example, endosperm has evolved to permit the accumulation of large amounts of storage proteins in a small volume and a stable environment. Moreover, the small size of the endosperm permits recombinant proteins to reach a relatively high concentration in a small biomass, which is beneficial for extraction and downstream processing. Such downstream processing is also simplified as a result of low levels of compounds known to interfere with downstream processing steps, such as phenolics and alkaloids present in tobacco leaves and oxalic acid present in alfalfa. Furthermore, because seed is generally suitable for human and animal consumption, accumulation of proteins in developing seed is an attractive means for producing recombinant proteins for oral delivery to humans or animals, e.g., for production of a foodstuff having a pharmaceutical quality, e.g., an oral vaccine or for production of a foodstuff having an improved nutritional quality.

Accumulation of proteins in the seed of a plant is also particularly useful as the harvesting of seed is already a major feature of crop based agriculture and is relatively easy to implement using existing techniques. The selective expression of proteins in endosperm, as opposed to constitutive expression throughout the plant, has a reduced risk of interfering with vegetative plant growth. Moreover, such limited expression limits contact with non-target organisms, such as microbes in the biosphere and leaf-eating herbivores (Stoger et al., Current Opinion in Biotechnology, 16: 167-173, 2005). There is an ongoing need for regulatory sequences that are capable of conferring expression selectively or specifically in the endosperm e.g., because the majority of sequences isolated to date are leaky or non-selective in so far as they confer expression more generally in vegetative or floral tissues or reproductive organs, mature seeds or embryonic tissues, and/or because they are not operable in different species or confer different patterns of expression across species.

Only a few endosperm promoters are known in the art, and these are mostly derived from a few abundantly-expressed storage protein genes. Because of the difficulty in expressing multiple genes in plants from the same promoter, the small number of available promoters makes it difficult to modify or improve plant endosperm by gene stacking i.e., the expression of multiple transgenes. For example, competition between cis-acting elements for regulatory DNA binding proteins can reduce promoter efficiency such that expression of multiple transgenes under the control of the same promoter in the same cell may be reduced compared to when different promoters are employed.

It will be apparent to the skilled artisan from the foregoing that the genetic manipulation of seed endosperm is beneficial to agriculture, in permitting the production of pharmaceuticals for human or veterinary use and/or for improving or altering the nutritional quality of a foodstuff produced from a plant. Accordingly, promoters that confer expression in developing endosperm are clearly desirable to provide these benefits.

Conventional techniques of molecular biology, recombinant DNA technology are described, for example, in the following texts:
(i) Sambrook, Fritsch & Maniatis, Molecular Cloning: A Laboratory Manual, Cold Spring Harbor Laboratories, New York, Second Edition (1989), whole of Vols I, II, and III;
(ii) DNA Cloning: A Practical Approach, Vols. I and II (D. N. Glover, ed., 1985), IRL Press, Oxford, whole of text;
(iii) Oligonucleotide Synthesis: A Practical Approach (M. J. Gait, ed., 1984) IRL Press, Oxford, whole of text, and particularly the papers therein by Gait, ppl-22; Atkinson et al., pp 35-81; Sproat et al., pp 83-115; and Wu et al., pp 135-151;
(iv) Nucleic Acid Hybridization: A Practical Approach (B. D. Hames & S. J. Higgins, eds., 1985) IRL Press, Oxford, whole of text;
(v) Perbal, B., A Practical Guide to Molecular Cloning (1984);

### SUMMARY OF INVENTION

In work leading up to the present invention, the present inventors sought to provide such an isolated promoter by employing microarray technology, and subsequently isolating promoter sequences conferring expression in developing endosperm cells. As exemplified herein, the inventors identified two wheat transcripts that are expressed in developing endosperm in a tissue-selective and development-selective manner.

The inventors also identified transcripts in rice, barley, maize and sorghum having similar expression profiles by homology searching using the wheat transcript sequences. To isolate the promoters regulating expression of the wheat and maize transcripts, the inventors amplified nucleic acid upstream of the coding regions in wheat and maize genomic DNA, respectively, using primers in polymerase chain reactions (PCR). As exemplified herein, variants of the wheat promoters were obtained from rice and maize genomes.

The inventors have also demonstrated that the exemplary wheat promoters of the present invention confer selective and possibly specific expression on reporter genes to which they are operably connected in the developing endosperm of transgenic wheat and maize e.g., in the period form about 5-10 days after pollination (DAP) to about least about 25 DAP.

The exemplified promoters and methods for their isolation as described herein are thus representative of a class of promoters that in their native context confer selective/specific endosperm expression on genes to which they are operably connected.

Accordingly, the present invention provides an isolated promoter or an active fragment or derivative thereof capable of conferring selective expression on a gene to which it is operably connected in the endosperm of a developing plant seed, wherein said promoter in its native context confers endosperm-selective expression or preferential endosperm expression on a genomic gene comprising a sequence selected from the group consisting of:

(i) a sequence set forth in SEQ ID NO: 1 or 2;
(ii) a sequence encoding a polypeptide having at least about 50% identity to a polypeptide encoded by SEQ ID NO: 1 or 2 wherein said polypeptide is expressed selectively in endosperm of developing seed;
(iii) a sequence that hybridizes under at least moderate stringency conditions to a sequence at (i) or (ii) or a complementary sequence thereto wherein said hybridising sequence is expressed selectively in endosperm of developing seed; and
(iv) a sequence having homology to a sequence at (i) or (ii) as determined by homology searching using the BLASTN algorithm e.g., with a nucleotide mismatch penalty (−q) of at least −1 wherein said homologous sequence is expressed selectively in endosperm of developing seed.

The isolated promoter, active fragment or derivative is at least capable of conferring endosperm-selective expression or preferential endosperm expression on a gene to which it is operably connected in developing seed of a monocotyledonous plant e.g., wheat, maize, rice, barley or sorghum. Other sources of the promoter of the invention than those specifically recited herein are not to be excluded.

It will also be apparent that the promoter, active fragment or derivative may be isolated from a monocotyledonous plant e.g., wheat, maize, rice, barley or sorghum. In one example. the isolated promoter, active fragment or derivative is capable of conferring endosperm-selective expression or preferential endosperm expression on a gene to which it is operably connected during the period of from about 5 days after pollination (DAP) to at least about 25 DAP. It is to be understood that this selective expression means that the gene to which the promoter, fragment or derivative is connected is not expressed at a detectable level of transcript and/or protein e.g., as determined by conventional methods of transcript profiling or Northern hybridisation or RT-PCR or by immunological methods such as ELISA or by determining enzyme activity, in one or vegetative tissues or organs and/or one or more reproductive tissues or organs and/or one or more floral tissues or organs. For example, the promoter of the present invention does not confer detectable expression as determined by such methods in leaf and/or root and/or node and/or stem internode and/or glume and/or anther and/or ovary and/or pollen and/or husk and/or silk and/or embryo and/or mature seed endosperm.

In another example, the isolated promoter, active fragment or derivative of the present invention confers, induces or activates endosperm-specific expression on a gene to which it is operably connected i.e., expression is strictly localized to the developing endosperm.

Sequence analysis indicates that, notwithstanding the generally low sequence identity between different promoters, the isolated promoters, active fragments and derivatives thereof provided in accordance with the present invention possess structurally-conserved features which may permit their characterization and identification as a genus or sub-genus of endosperm-selective or endosperm-specific regulatory sequences. In one example, a promoter of the present invention comprises one or more nucleotide sequences set forth in Table 4 and/or Table 5 and/or Table 6 and/or Table 7 and/or Table 8 e.g., as determined by PLACE analysis of the regulatory sequences to identify cis-acting elements therein. In another example, an isolated promoter of the present invention comprises one or more nucleotide sequences as set forth in Table 1 i.e., corresponding to cis-acting elements conserved between five exemplified endosperm regulatory sequences. In yet another example, an isolated promoter of the present invention comprises a plurality of each element in the group consisting of an ARR1AT element, an ACGTATERD1 element, a CAATBOX1 element, a CACFTPPCA1 element, a CURECORECR element, a DOFCOREZM element, an EBOXBNNAPA element, a GATABOX element, a GT1CONSENSUS element, a GTGANTG10 element, and a MYCCONSENSUSAT element in the proximal 750 bp upstream of the translation start site of the corresponding genomic gene from which it is derived. In accordance with this example, each such element may be represented at least 2 or 3 or 4 or 5 or 6 times in the proximal 750 bp upstream of the translation start site of the corresponding genomic gene from which it is derived. Alternatively, or in addition, CACFTPPCA1 elements, DOFCOREZM elements and GT1CONSENSUS elements are also each represented at least 4 times in the proximal 750 bp upstream of the translation start site of the corresponding genomic gene from which the promoter is derived. Alternatively, or in addition, ARR1AT elements, CURECORECR elements, DOFCOREZM elements, EBOXBNNAPA elements, GTGANTG10 elements and MYCCONSENSUSAT elements are each represented at least 4 times in the proximal 750 bp upstream of the translation start site of the corresponding genomic gene from which the promoter is derived. Alternatively, or in addition, the isolated promoter, active fragment or derivative further comprises at least one element in the group consisting of an IBOXCORE element, a MYB2CONSENSUS element, a MYBCORE element and a WRKY71OS element in the proximal 750 bp upstream of the translation start site of the gene to which the promoter is operably connected in its native context. At least one element in the group consisting of a MYBSTI element, a MYBCOREATCYCB1 element and a PRECONSCRHSP70A element may also be represented in the proximal 750 bp upstream of the translation start site of the gene to which the promoter is operably connected in its native context.

A promoter of the present invention can thus comprise one or multiple copies of a sequence set forth in Tables 1 or 4-8 e.g., repeated in the promoter sequence with or without intervening sequences such as tandem repeat sequences, and/or in the opposing orientation e.g., in different species or alleles. A promoter of the present invention may also include reverse complement sequences of any sequence set forth in Tables 1 or 4-8 infra. e.g., in different species or alleles.

The sequences presented in Table 1 that are conserved across species, or between different homeologues or alleles within a species, can individually or collectively contribute to the expression of pattern of expression conferred by the promoter of the present invention, thereby explaining one or more conserved patterns of expression observed for the transcript operably connected to the promoter in different or the same species. Accordingly, representative examples of the promoter of the present invention, other than those examples arising by gene duplication, have low sequence identity overall notwithstanding conserved ability to confer expression in a particular temporal or spatial pattern and/or in response to one or more signals, e.g., environment, hormone, etc.

Those skilled in the art will also be aware that such short sequences are useful for conferring expression or a pattern of expression on a heterologous nucleic acid to which it is operably connected e.g., to activate, silence, enhance, repress or otherwise modulate expression and/or cell-type-specificity and/or developmental specificity of a nucleic acid to which it is operably connected.

In yet a further example, the isolated promoter, active fragment or derivative according comprises a nucleotide sequence selected from the group consisting of:

(i) a sequence selected from the group consisting of SEQ ID NOs: 3, 4, 5, 6, 7 and 8;
(ii) a sequence complementary to a sequence at (i);
(iii) a sequence having at least about 70% sequence identity to a sequence of (i) or (ii); and
(iv) a sequence amplifiable from genomic DNA using one or more amplification primers wherein each of said primers comprises a sequence of at least about 12 contiguous nucleotides in length derived from SEQ ID NO: 1 or 2 or a complementary sequence thereto.

For the purposes of nomenclature, the sequence set forth in SEQ ID NO: 3 comprises the promoter designated “WP05” from wheat that regulates endosperm expression of the genomic gene equivalent of the transcript set forth in SEQ ID NO: 1 or a homolog thereof in its native context. The sequence set forth in SEQ ID NO: 4 comprises a 2400 bp variant of the promoter designated “WP07” from wheat that regulates endosperm expression of the genomic gene equivalent of the transcript set forth in SEQ ID NO: 2 or a homolog thereof in its native context. The sequence set forth in SEQ ID NO: 5 comprises a 2066 bp variant of the promoter designated “WP07” from wheat that regulates endosperm expression of the genomic gene equivalent of the transcript set forth in SEQ ID NO: 2 or a homolog thereof in its native context. The sequence set forth in SEQ ID NO: 6 comprises a 330 bp 5′-upstream regulatory sequence of the rice gene locus designated “LOC_Os01 g01290.1” in its native context, wherein said rice gene is expressed in developing seed and identified by homology searching as described in the examples hereof. The sequence set forth in SEQ ID NO: 7 comprises a 5′-upstream regulatory sequence of the maize gene locus designated “ZmGSStuc11-12-04.64626.1” in its native context, wherein said maize gene is expressed in developing seed and identified by homology searching as described in the examples hereof. The sequence set forth in SEQ ID NO: 8 comprises a 5′-upstream regulatory sequence of the maize gene locus designated “ZmGSStuc11-12-04.16895.1” in its native context, wherein said maize gene is expressed in developing seed and identified by homology searching as described in the examples hereof. It is to be understood that the present invention clearly encompasses an isolated promoter, active fragment or derivative according comprising a nucleotide sequence selected individually or collectively from the group consisting of:

(i) a sequence selected from the group consisting of SEQ ID NOs: 3, 4, 5, 6, 7 and 8; and
(ii) a sequence complementary to any one or more of the sequences at (i).

It is also to be understood that the present invention extends nzutatis mutandis to an isolated promoter or an active fragment or derivative thereof comprising a sequence of nucleotides that in its native context confers endosperm expression on nucleic acid encoding a polypeptide encoded by SEQ ID NO: 1 or 2 or LOC_Os01 g01290.1 or ZmGSStuc11-12-04.64626.1 or ZmGSStuc11-12-04.16895.1 or a homolog of any one or more of said nucleic acid. Alternatively, or in addition, the promoter of the present invention will comprise a sequence that in its native context confers endosperm-selective or endosperm-specific expression on nucleic acid that hybridizes under at least moderate stringency conditions, and preferably high stringency conditions, to nucleic acid encoding a polypeptide encoded by SEQ ID NO: 1 or 2 or LOC_Os01g01290.1 or ZmGSStuc11-12-04.64626.1 or ZmGSStuc11-12-04.16895.1.

Alternatively, or in addition, the promoter of the present invention will comprise a sequence that in its native context confers endosperm-selective or endosperm-specific expression on nucleic acid that hybridizes under at least moderate stringency conditions, and preferably high stringency conditions, to a complement of nucleic acid encoding a polypeptide encoded by SEQ ID NO: 1 or 2 or LOC_Os01g01290.1 or ZmGSStuc11-12-04.64626.1 or ZmGSStuc11-12-04.16895.1.

Hybridization conditions will be known to the skilled artisan or are described herein. Due to the recognized low overall sequence identity between functionally-related promoters, low stringency hybridization conditions are preferred, however moderate or high stringency may be employed.

More preferably, a promoter of the present invention or an active fragment or derivative thereof comprises a nucleotide sequence that is amplifiable from genomic. DNA using one or more amplification primers wherein each of said primers comprises a sequence of at least about 12 contiguous nucleotides in length derived from a sequence set forth in SEQ ID NO: 1 or 2 or LOC_Os01 g01290.1 or ZmGSStuc11-12-04.64626.1 or ZmGSStuc11-12-04.16895.1, or a complementary sequence thereto.

In a particularly, preferred embodiment, a promoter of the present invention comprises a sequence selected from the group consisting of SEQ ID NO: 3, 4, 5, 6, 7, and 8, or a complementary sequence thereto or an active fragment or derivative of said sequence or complementary sequence.

The present invention also provides the use of a promoter as described herein according to any embodiment or an active fragment or derivative thereof in the production of an expression construct.

For example, a promoter of the present invention is particularly useful for the production of an expression construct for expressing nucleic acid to which it is operably connected in cells of developing endosperm, and preferably being preferentially or selectively expressed in endosperm and cells and tissues thereof.

The term “expression construct” is to be taken in its broadest context and includes an isolated promoter or active fragment or derivative placed in operable connection with a transgene.

As used herein, the term “transgene” shall be taken to mean nucleic acid other than that upon which the promoter of the invention confers expression or a pattern of expression in its native context i.e., “heterologous nucleic acid”. The general applicability of the present invention is not to be limited by the nature of the transgene. Suitable transgenes will be apparent to the skilled artisan based on the description herein, and include a nucleic acid encoding a polypeptide to be expressed in a developing endosperm or cell or tissue thereof or a nucleic acid capable of reducing expression of a nucleic acid in a developing endosperm or cell or tissue thereof, e.g., a short interfering RNA (siRNA) or RNAi or antisense RNA or micro RNA (miRNA). Preferably, the nucleic acid is capable of modulating expression of a polypeptide involved in endosperm development, starch or storage protein accumulation or biosynthesis or in conferring disease resistance or nutritional value on the seed. It will be understood from the foregoing that it is preferred for such expression to be modulated by virtue of the promoter conferring expression in the context of one or more factors required for expression, repression, inhibition or reduction to occur. Preferably, expression is modulated preferentially or selectively under these conditions. Additional suitable transgenes will be apparent to the skilled artisan based on the description herein, and clearly include transgenes encoding a polypeptide that confers a nutritional or pharmaceutical quality on a developing endosperm or encoding a polypeptide for production of a useful downstream product or bi-product e.g., starch, brewed or fermented beverages or foods, flour, flour-containing products such as bread, biscuits, pasta or noodles, starches, fatty acids, edible oils, paper, textiles, ethanol, polymers or other industrial application(s).

The present invention also provides a method for producing an expression construct, said method comprising linking a promoter of the present invention or active fragment or derivative as described herein according to any embodiment to a transgene such that the promoter is capable of conferring expression or a pattern of expression on said transgene in developing endosperm or a cell or tissue thereof.

Preferred cells tissues or organs for performing this embodiment are plant cells, tissues or organs, e.g., monocotyledonous plant cells, tissue or organs, such as from wheat, barley, maize, rice, sorghum, rye, millet (e.g. pearl millet or proso millet), buckwheat (e.g., of the family Polygonaceae), oat (e.g., Avena sativa) or a cell, tissue or organs from any other plant from the family Graminaceae, Gramineae or Poaceae. This includes any plant cell, tissue or organ having the ability to confer expression on the nucleic acid to which the promoter is operably-connected in its native context as herein before defined.

Preferred linkages between the promoter, active fragment or derivative and the transgene are covalent linkages. It is to be understood that, because the promoter, active fragment or derivative may confer expression at some distance from a transgene to which it is operably connected, the transgene need not be juxtaposed to the promoter, active fragment or derivative, i.e., there may be intervening sequence of up to about 2 kb in length, preferably up to about 1 kb in length, more commonly about 200-500 bp in length. Shorter intervening sequences such as the sequence of an intron of up to about 100 or 200 bp in length may also be employed.

Suitable methods for linking nucleic acids will be apparent to the skilled artisan and/or described herein and include enzymatic ligation, e.g., T4 DNA ligase, topoisomerase-mediated ligation e.g., using Vaccinia DNA topoisomerase I, recombination in cis or trans, e.g., using a recombinase or by random integration, amplification from one or more primer sequences including primer extension means, amplification from a vector, or chemical ligation, e.g., cyanogen bromide-mediated condensation of nucleic acids.

In a further example the present invention also provides an expression construct comprising a promoter of the present invention as described herein according to any embodiment operably connected to a transgene.

The present invention also provides the use of a promoter as described herein according to any embodiment or an active fragment or derivative thereof in the production of an expression vector. Preferably, the promoter is used operably linked to a transgene. The skilled artisan will be aware that an expression vector comprises sufficient genetic information to permit expression to be initiated from a promoter or active fragment or derivative e.g., by virtue of the presence of the promoter, active fragment or derivative and one or more transcription termination sequences and/or enhancer element sequences and/or intron sequences and/or intron splice junction sequences in operable connection therewith. An expression vector will generally also include one or more sequences to permit it to be maintained in a cell e.g., one or more selectable marker genes e.g., to confer antibiotic or herbicide resistance on cells comprising the expression construct, and one or more origins of replication e.g., for replication in bacterial cells or yeasts. An expression vector may also include one or more recombinase site sequences to permit excision of a portion of its DNA in a cell and/or to facilitate integration into host cell DNA.

The present invention also provides a method for producing an expression vector, said method comprising linking a promoter of the present invention or active fragment or derivative as described herein according to any embodiment to an empty vector to thereby produce an expression vector. As used herein, the term “empty vector” shall be taken to mean a vector without a promoter of the present invention or an active fragment or derivative thereof. The skilled artisan will be aware that exemplary vectors include plasmids, phagemids, cosmids, viral genome or subgenomic fragment, phage artificial chromosomes e.g., P1 artificial chromosomes, bacterial artificial chromosomes, yeast artificial chromosomes, or other nucleic acid capable of being maintained chromosomally or extra-chromosomally and/or replicating in a cell.

In one example, the process additionally comprises linking a transgene to the expression vector such that the promoter, active fragment or derivative and the transgene are in operable connection.

In a further alternative, the present invention provides a process for producing an expression vector, said method comprising linking an expression construct as described herein according to any embodiment to an empty vector to thereby produce an expression vector.

In the present context, the linkages between the various components of the expression vector and the means for achieving such linkage will be understood to be the same as for producing an expression construct of the present invention.

In one example, the method additionally comprises producing or obtaining an expression construct of the present invention.

In another example, the method comprises obtaining a promoter, active fragment or derivative of the invention and/or a transgene and/or an empty vector for use in producing an expression vector of the invention.

In a further example, the present invention also provides an expression vector comprising a promoter of the present invention or active fragment or derivative thereof.

Preferred expression vectors will comprise an expression construct of the present invention i.e., including a promoter of the present invention operably connected to a transgene. For example, the inventors have produced vectors for biolistic or Agrobacterium-mediated transformation of wheat, e.g., comprising a sequence set forth in SEQ ID NO: transformation of wheat, e.g., comprising a sequence set forth in SEQ ID NOs: 10-17, or for Agrobacterium-mediated transformation of maize, e.g., comprising a sequence set forth in SEQ ID NO: 18 or 19.

A promoter as described herein according to any embodiment or an active fragment or derivative thereof is also useful for the production of a transgenic plant or plant part, e.g., comprising a promoter, active fragment or derivative of the invention in operable connection with a transgene or in operable connection with an endogenous nucleic acid. By “endogenous nucleic acid” is meant nucleic acid of nuclear or organellar origin in a plant, plant cell or plant part that is made transgenic by virtue of the introduction of the promoter, active fragment or derivative. For example, such “endogenous nucleic acid” occurs naturally in the plant or plant part that is made transgenic by virtue of the introduction of a promoter, active fragment or derivative of the invention.

Accordingly, the present invention provides for use of a promoter, active fragment or derivative of the present invention in the production of a plant cell, plant tissue, plant organ or whole plant, e.g., for modulating endosperm expression of a transgene i.e., conferring expression on an endogenous or heterologous transgene preferentially or selectively in developing endosperm and/or for repressing or reducing expression of an endogenous transgene in developing endosperm.

The term “plant part” is to be understood to mean a cell, tissue or organ of a plant, or plurality of cells, tissues or organs of a plant, including any reproductive material e.g., seed, developing endosperm optionally including scutellum and/or aleurone and preferably developing endosperm. Preferred plant parts of the present invention comprise a promoter of the invention or active fragment or derivative thereof.

Alternatively, the present invention provides for use of a promoter, active fragment or derivative of the present invention in the preparation of an expression vector or expression construct for producing a plant cell, tissue or organ or whole plant, e.g., for conferring expression preferentially or selectively in developing endosperm optionally including aleurone and/or scutellum and/or for repressing or reducing expression in developing endosperm optionally including aleurone and/or scutellum.

In one example, a promoter, active fragment or derivative of the present invention is used to produce a plant or plant part in which the expression of an endogenous nucleic acid is altered, i.e., the promoter, active fragment or derivative is operably connected to an endogenous nucleic acid. For example, production of such a plant part or plant permits the expression of an endogenous nucleic acid to be enhanced or reduced. Such modulated expression is useful for, for example, inducible production of an expression product of interest, e.g., a protein of interest or for controlling the timing and/or location of expression of an expression product of interest, or for reducing levels of undesirable expression products or delaying their expression.

Alternatively, a promoter, active fragment or derivative is used to identify and/or isolate a nucleic acid that induces a phenotype of interest. For example, the promoter, active fragment or derivative is introduced into the genome of a plant or plant part such that it is operably connected to genomic nucleic acid to thereby produce a phenotype in said plant or plant part different to the phenotype of otherwise isogenic or near isogenic material lacking said promoter, active fragment or derivative at that genomic location. The nucleic acid operably linked to the promoter, active fragment or derivative in the genome of the plant is optionally identified and/or isolated using standard techniques, e.g., 5′ rapid amplification of cDNA ends (RACE) or 3′ RACE.

In another example, a promoter, active fragment or derivative of the present invention is used to confer expression as hereinbefore defined on a transgene in a plant part. It is to be understood that an expression construct or expression vector of the present invention is also used to produce a plant cell, plant part or whole plant for the purpose of conferring expression as hereinbefore defined on a plant part. In the case of a transgenic plant or a transgenic plant cell or a transgenic plant part comprising an expression construct, the expression construct can be integrated into the genome of the plant, plant cell or plant part or can be in an episome or is extra-chromosomal.

Preferably, a promoter, active fragment, derivative, expression construct or expression vector of the present invention is used to produce a plant or plant part having an altered phenotype compared to an otherwise isogenic plant part or plant not having the promoter, active fragment, derivative expression vector or expression construct. For example, a transgenic plant or plant part comprises an expression construct or expression vector of the present invention comprising a transgene or structural gene placed operably under control of a promoter of the present invention.

In one example, the open reading frame of a structural gene to be expressed under control of a promoter of the present invention confers or enhances disease or pest tolerance on a plant (e.g., an open reading frame from an insect resistance gene, a bacterial disease resistance gene, a fungal disease resistance gene, a viral disease resistance gene, a nematode disease resistance gene). In another example, the open reading frame of a structural gene to be expressed under control of a promoter of the present invention confers or enhances herbicide tolerance on a plant (e.g., a glyphosate resistance gene or phosphinothricin resistance gene). In another example, the open reading frame of a structural gene to be expressed under control of a promoter of the present invention modifies grain composition or quality, such as endosperm size, endosperm cell number, seed size, or other yield characteristic). In yet further examples, the open reading frame of a structural gene to be expressed under control of a promoter of the present invention modifies nutrient utilization, improves tolerance to a mycotoxin, improves or enhances environmental or other stress tolerance resistance (e.g., a drought tolerance gene, heat tolerance gene, cold tolerance gene, frost tolerance gene, flooding tolerance gene, salt tolerance gene, or oxidative stress tolerance gene), oil quantity and/or quality, amino acid or protein composition, and genes for expression of exogenous products such as enzymes, cofactors, and hormones from plants, other eukaryotes or prokaryotic organisms. Commercial traits in plants are also created through the modified expression of genes that alter starch or protein for the production of paper, textiles, ethanol, polymers or other materials with industrial uses.

In another example, the expression of an endogenous endosperm gene is reduced using a promoter of the present invention e.g., by means of expressing one or more transgenes comprising one or more antisense molecules, ribozymes (Haseloff et al. Nature 334, 585-591, 1988; Steinecke et al. EMBO J. 11, 1525 (1992); Perriman et al., Antisense Res. Dev. 3, 253 (1993)), co-suppression molecules, RNAi molecules (Napoli et al. Plant Cell 2, 279-289, 1990; U.S. Pat. No. 5,034,323; Sharp et al., Genes Dev. 13, 139-141, 1999; Zamore et al., Cell 101, 25-33, 2000; and Montgomery et al., PNAS USA 95, 15502-15507, 1998), hairpin structures (Smith et al. Nature 407, 319-320, 2000; WO 99/53050; and WO 98/53083), microRNAs (Aukerman et al., Plant Cell 15, 2730-2741, 2003), transcription factor-targeted genes (e.g., WO 01/52620; WO 03/048345; and WO 00/42219), repressor-encoding genes, transposons, or dominant-negative mutants in the endosperm under operable control of the promoter of the invention. The present invention clearly encompasses the use of other methods or combinations of any two or more of the above procedures known to those of skill in the art.

A promoter of the present invention or active fragment or derivative thereof has particular utility for modifying one or more grain traits by expressing a structural gene e.g., an open reading frame, or molecule to effect reduced transcription of an endogenous endosperm gene to which it is operably connected. Preferred grain traits include e.g., fatty acid content and/or composition, amino acid content and/or composition including the content of lysine-containing or sulfur-containing proteins and the content and/or composition of seed storage proteins, starch content and/or composition, growth regulatory proteins including cell cycle regulatory proteins, apoptosis or kernel abortion, and environmental stress genes. In another example, the transgene encodes a siRNA or antisense RNA or RNAi or miRNA that inhibits expression of a polypeptide in developing endosperm. Alternatively, the nucleic acid encodes an antibody fragment capable of binding to and inhibiting activity of a polypeptide in developing endosperm.

In a further example, a promoter, active fragment or derivative or expression construct or expression vector of the present invention is used to confer resistance to a disease or pest on a plant part or a whole plant. For example, an expression construct or expression vector comprises a transgene confers resistance to a plant disease or a plant pest when expressed such as a chitinase or a thaumatin-like protein, e.g., from wheat, or a coat protein from a pest (e.g., a barley yellow mosaic virus coat protein).

In a still further example, a transgene confers a pharmaceutical quality on a plant or plant part in which it is expressed. For example, the transgene encodes an immunogenic protein, such as, for example, a hepatitis B surface antigen.

The present also encompasses a use of a promoter, active fragment, derivative, expression construct or expression vector of the present invention to confer a nutritional quality on a plant or plant part. For example, an expression construct or expression vector comprises a transgene encoding a seed storage protein, a fatty acid pathway enzyme, a tocopherol biosynthetic enzyme, an amino acid biosynthetic enzyme or a starch branching enzymes. In one example, the transgene encodes a Brazil nut protein, a calcium-binding protein or an iron-binding protein.

The present also encompasses a use of a promoter, active fragment, derivative, expression construct or expression vector of the present invention to modify morphology of a plant or plant part. For example, an expression construct or expression vector comprises a transgene encoding a polypeptide involved in auxin synthesis or metabolism or cytokinin synthesis or metabolism (e.g., cytokinin oxidase). By altering the level of auxin and/or cytokinin in a plant or plant part, the morphology of the plant or plant part is modified.

It is to be understood that the promoter of the present invention has particular utility for the purposes of gene stacking, such as when used with a different promoter to express a plurality of structural genes or transgenes in the endosperm of a plant. In a further example, the promoter of the present invention is used in conjunction with one or more other promoters to express a plurality of structural genes or transgenes in the same or a different cell of the plant e.g., wherein such expression is simultaneous, contemporaneous or synchronous. For example, the promoter of the present invention or an active fragment or derivative thereof is utilized to express different structural genes or transgenes that, when expressed, modify the same biochemical pathway in the plant seed. Alternatively, the promoter of the present invention or an active fragment or derivative thereof is utilized to express functionally distinct or unrelated structural gene or transgene to a structural gene or transgene expressed under control of the other promoter in the plant seed. As will be known to the skilled artisan, gene stacking may be performed by simultaneous or sequential transformation processes involving the introduction of gene constructs to be expressed.

In one example of gene stacking, a construct comprising the promoter of the present invention or an active fragment or derivative thereof operably linked to a transgene or structural gene is introduced to plant endosperm that already expresses a transgene or structural gene under control of another promoter that confers or regulates expression in a number of different plant organs, tissues or cells, e.g., including the endosperm. In another example, a two component system is employed wherein two parent lines are produced each of which expresses a desired transgene under the control of a promoter such that one plant line comprises a promoter, active fragment or derivative thereof in accordance with the present invention and the other plant line comprises the other promoter and wherein the two transgenic plant lines are crossed to produce a progeny plant expressing both transgenes. In another example, a first construct comprising the promoter of the present invention or an active fragment or derivative thereof operably linked to a transgene or structural gene is introduced to plant endosperm alongside a second construct comprising a transgene or structural gene operably linked to a different promoter that confers or regulates expression in a number of different plant organs, tissues or cells, e.g., including the endosperm. Exemplary promoters that confer or regulate expression in a number of different plant organs, tissues or cells, e.g., including the endosperm are known in the art e.g., the p326 promoter, YP0144 promoter, YP0190 promoter, p13879 promoter, YP0050 promoter, p32449 promoter, 21876 promoter, YP0158 promoter, YP0214 promoter, YP0380 promoter, PT0848 promoter, PT0633 promoter, CaMV 35S promoter, mannopine synthase (MAS) promoter, the 1′ or 2′ promoters derived from T-DNA of Agrobacterium tumefaciens, figwort mosaic virus 34S promoter, actin promoters such as from rice, and ubiquitin promoter such as from maize (Ubi-1).

In another example of gene stacking, a construct comprising the promoter of the present invention or an active fragment or derivative thereof operably linked to a transgene or structural gene is introduced to plant endosperm that already expresses a transgene or structural gene under control of a mature endosperm promoter that confers or regulates expression in maturing endosperm albeit not necessarily exclusively or predominantly in the maturing endosperm. In another example, a two component system is employed wherein two parent lines are produced each of which expresses a desired transgene under the control of a promoter such that one plant line comprises a promoter, active fragment or derivative thereof in accordance with the present invention and the other plant line comprises the other promoter active in maturing endosperm and wherein the two transgenic plant lines are crossed to produce a progeny plant expressing both transgenes in the endosperm. In yet another example, a first construct comprising the promoter of the present invention or an active fragment or derivative thereof operably linked to a transgene or structural gene is introduced to plant endosperm alongside a second construct comprising a transgene or structural gene operably linked to a different promoter that confers or regulates expression in maturing endosperm albeit not necessarily exclusively or predominantly in the maturing endosperm.

In another example of gene stacking, a construct comprising the promoter of the present invention or an active fragment or derivative thereof operably linked to a transgene or structural gene is introduced to plant endosperm that already expresses a transgene or structural gene under control of a mature endosperm promoter that confers or regulates expression in the embryo sac or early endosperm albeit not necessarily exclusively or predominantly in the embryo sac/early endosperm. In yet another example, a first construct comprising the promoter of the present invention or an active fragment or derivative thereof operably linked to a transgene or structural gene is introduced to plant endosperm alongside a second construct comprising a transgene or structural gene operably linked to a different promoter that confers or regulates expression in embryo sac or early endosperm albeit not necessarily exclusively or predominantly in the embryo sac/early endosperm. By “embryo sac” or “early endosperm” is meant the polar nuclei and/or the central cell, or in precursors to polar nuclei and preceding cellularization. Exemplary promoters that are active in embryo sac or early endosperm include e.g., the Arabidopsis viviparous-1 gene promoter (see, GenBank No. U93215); the Arabidopsis Atmyc1 gene promoter (Urao et al., Plant Mol. Biol., 32: 571-57, 1996; Conceicao Plant, 5, 493-505, 1994); the Arabidopsis FIE gene promoter (see GenBank No. AF129516); the Arabidopsis MEA gene promoter; the Arabidopsis FIS2 gene promoter (see GenBank No. AF096096); the Arabidopsis FIE 1.1 gene promoter (U.S. Pat. No. 6,906,244), the maize MAC1 gene promoter (Sheridan et al., Genetics, 142, 1009-1020, 1996); and the maize Cat3 gene promoter (see GenBank No. L05934; Abler et al., Plant Mol. Biol., 22, 10131-1038), 1993.

The present invention also provides a method for producing a transgenic plant cell, said method comprising introducing a promoter, active fragment or derivative of the present invention or an expression construct or expression vector of the present invention into the plant cell. Suitable methods for introducing a nucleic acid into a plant cell will be apparent to the skilled artisan, e.g., transformation using CaCl₂ and variations thereof, PEG-mediated uptake to protoplasts, microparticle bombardment, electroporation, microinjection, vacuum-infiltration of tissue or Agrobacterium-mediated transformation. For example, a transgenic plant cell is produced by performing a method of Agrobacterium-mediated transformation as described in International Patent Application No. PCT/AU2007/000021.

Preferably, the method additionally comprises producing, providing or obtaining the promoter, active fragment, derivative, expression construct or expression vector.

In one example, a method for producing a transgenic plant cell of the present invention additionally comprises contacting the produced transgenic plant cell with a compound that induces callus formation and/or induces dedifferentiation of the transgenic cell (or a cell derived therefrom) and/or induces the production of an undifferentiated cell from said transgenic cell for a time and under conditions sufficient to produce a callus and/or dedifferentiated cell and/or undifferentiated cell. A suitable compound will be apparent to the skilled artisan e.g., a synthetic or natural auxin such as, for example, a compound selected from the group consisting of 2,4-dichlorophenoxyacetic acid, 3,6-dichloro-o-anisic acid, 4-amino-3,5,6-trichloropicolinic acid and mixtures thereof. By “callus” is meant a cluster or group of undifferentiated cells resulting from cell division in the absence of regeneration.

Those skilled in the art are aware that a transgenic plant cell can be used without undue experiment to produce a transgenic plant, e.g., by regeneration. By “regeneration” is meant a process by which a plant or plant part, especially a plantlet, is produced from a transgenic plant cell e.g., by a process of organogenesis or embryogenesis.

As used herein, the term “organogenesis” shall be taken to mean a process by which shoots and roots are developed sequentially from meristem centres.

As used herein, the term “embryogenesis” shall be taken to mean a process by which shoots and roots develop together in a concerted fashion (not sequentially), whether from somatic cells or gametes.

As used herein, the term “plantlet” shall be taken to mean a shoot or root that has developed from a plant cell, e.g., using in vitro techniques. For example, a plantlet is a shoot or root that has been induced to grow from a callus using a compound, such as, for example, indole-3-acetic acid, benzyladenine, indole-butyric acid, zeatin, α-naphthaleneacetic acid, 6-benzyl aminopurine, thidiazuron or kinetin, 2iP.

Based on the foregoing description, it will be apparent to the skilled artisan that the present invention provides for use of a transgenic plant cell comprising a promoter, active fragment, derivative, expression construct or expression vector of the present invention for the production of a transgenic plant or plantlet.

The present invention also provides a method for producing a transgenic plant or plantlet, said process comprising:

(i) providing, producing or obtaining a transgenic plant cell or callus comprising a promoter, active fragment, derivative, expression construct or expression vector of the present invention; and
(ii) regenerating a transgenic plant or plantlet from the transgenic plant cell or callus at (i), thereby producing a transgenic plant or plantlet.

In one example, the method is for producing a transgenic plant or plantlet in which a promoter, active fragment or derivative of the present invention confers expression as hereinbefore defined on a nucleic acid, e.g., a transgene, preferentially or selectively in developing endosperm optionally including aleurone and/or scutellum and/or for repressing or reducing expression of a nucleic acid preferentially or selectively in a developing endosperm.

Methods for regenerating a plant or plantlet from a plant cell or callus will be apparent to the skilled artisan and/or described herein. For example, a transgenic plant cell is contacted with a compound that induces callus formation and/or induces dedifferentiation of the transgenic cell (or a cell derived therefrom) and/or induces the production of an undifferentiated cell from said transgenic cell for a time and under conditions sufficient to produce a callus and/or dedifferentiated cell and/or undifferentiated cell, e.g., a compound described supra. Callus is generally contacted with a compound that induces shoot and/or root formation, e.g., a compound described supra for the production of a plantlet for a time and under conditions for a plantlet to form. To produce a whole plant a plantlet is grown for a time and under conditions for it to develop into a whole plant (e.g., grow to maturity).

In one example, the method for producing a transgenic plant or plantlet as described herein according to any embodiment additionally comprises providing or obtaining from the transgenic plant or plantlet, an offspring plant and/or seed and/or propagating material and/or reproductive material and/or germplasm, wherein said offspring plant, seed, propagating material or reproductive material comprises a promoter, active fragment, derivative, expression construct or expression vector of the present invention.

The present invention additionally provides a method for producing a transgenic seed from a plant, said method comprising providing, producing or obtaining a transgenic plant or plantlet as described herein according to any embodiment and growing or maintaining the transgenic plant or plantlet for a time and under conditions sufficient for seed to be produced. Optionally, the method additionally comprises obtaining seed comprising the introduced promoter, active fragment or derivative of the invention or expression construct or expression vector of the invention.

The present invention also provides a transgenic plant or plantlet or plant part or offspring plant or seed or propagating material or reproductive material or germplasm comprising a promoter, active fragment, derivative, expression construct or expression vector of the present invention. In one example, the plant or plantlet or plant part or offspring plant or seed or propagating material or reproductive material or germplasm comprises a promoter, active fragment or derivative operably connected to an endogenous nucleic acid of said plant or plantlet or plant part or offspring plant or seed or propagating material or reproductive material or germplasm.

In a preferred embodiment, the present invention provides a transgenic plant or plantlet or plant part or offspring plant or seed or propagating material or reproductive material or germplasm comprising a nucleic acid in operable connection with a promoter, active fragment or derivative of the present invention, e.g., comprising an expression construct or expression vector of the present invention. Preferably, the promoter, active fragment or derivative confers expression on the nucleic acid preferentially or selectively in developing endosperm and/or represses or reduces expression of the nucleic acid preferentially or selectively in developing endosperm.

The present invention additionally provides for use of a transgenic plant, plantlet or plant part for the production of a zygote and/or an offspring plantlet and/or an offspring plant.

Additionally, the present invention provides a method for breeding a transgenic plant. The term “breeding” is to be taken in its broadest context to mean any process by which a zygote and/or an offspring plantlet or plant is produced from or using a parent plant a part thereof or a cell thereof. For example, the term “breeding” encompasses sexual reproduction such as, cross-breeding or cross-pollination, whereby reproductive material, e.g., pollen from one plant is used to fertilize reproductive material, e.g., an egg cell within an ovule from another plant. The term “breeding” also encompasses sexual reproduction such as selfing or self-fertilization, whereby reproductive material from a plant, e.g., pollen is used to fertilize reproductive material, e.g., an egg cell within an ovule, from the same plant. The term “breeding” also encompasses vegetative forms of reproduction, such as the production of a plant from a stolon or a rhizome or a bulb or a tuber or a corm or a cutting or a graft or a bud. The term “breeding” also encompasses in vitro methods, e.g., in vitro fertilization and zygote culture.

In the case of sexual reproduction, the present invention provides a method for breeding a transgenic plant, said method comprising:

(i) providing, producing or obtaining a transgenic plant comprising a promoter, active fragment, derivative, expression construct or expression vector of the present invention; and
(ii) breeding the transgenic plant produced at (i) to thereby produce a zygote comprising a promoter, active fragment, derivative, expression construct or expression vector of the present invention.

Alternatively, the method comprises:

(i) providing, producing or obtaining plant reproductive material comprising a promoter, active fragment, derivative, expression construct or expression vector of the present invention; and
(ii) combining reproductive material of a plant with the reproductive material at (i) such that a zygote comprising a promoter, active fragment, derivative, expression construct or expression vector of the present invention is produced.

Preferably, the method additionally comprises growing the zygote to form a transgenic developing endosperm and/or a transgenic plantlet and/or a transgenic plant and/or a transgenic plant part, e.g., developing endosperm.

In one example, the step of obtaining a transgenic plant supra, comprises obtaining a seed or a plantlet or a pant part comprising a promoter, active fragment, derivative, expression construct or expression vector of the present invention, and growing said seed plantlet or plant or plant part to thereby obtain the transgenic plant.

In the case of cross-breeding, the transgenic plant is bred with or transgenic reproductive material is combined with a transgenic plant or transgenic reproductive material to produce a zygote, plant, plantlet or plant part homozygous or heterozygous for a promoter, active fragment, derivative, expression construct or expression vector of the present invention. Alternatively, the transgenic plant is bred with or transgenic reproductive material is combined with a wild-type plant or wild-type reproductive material to produce a zygote, plant, plantlet or plant part heterozygous for a promoter, active fragment, derivative, expression construct or expression vector of the present invention.

Preferably, a method of breeding of the present invention additionally comprises selecting or identifying a zygote, plantlet, plant part or whole plant comprising a promoter, active fragment, derivative, expression construct or expression vector of the present invention.

In one example, a method of breeding of the present invention additionally comprises detecting expression or a pattern of expression of a nucleic acid operably connected to a promoter, active fragment or derivative of the present invention in a plantlet, plant part or whole plant.

In the case of vegetative reproduction, the present invention provides a method comprising:

(i) providing, producing or obtaining a transgenic plant, plantlet or plant part comprising a promoter, active fragment, derivative, expression construct or expression vector of the present invention; and
(ii) maintaining the transgenic plant for a time and under conditions sufficient for the plant to reproduce vegetatively.

Suitable conditions will depend on the form of vegetative reproduction and will be apparent to the skilled artisan. For example, a lateral shoot from a plant is induced to form adventitious roots by burying the shoot and, following adventitious root formation, the shoot is separated from the parent plant and a new plant grown. Alternatively, or in addition, a plant or plantlet or plant part is induced to form a callus, e.g., by cutting a part of the plant, plant part or plantlet or using a process described supra, and the callus maintained under conditions sufficient to a plantlet or plant to grow.

As exemplified herein, a promoter as described herein according to any embodiment is useful for expressing a nucleic acid in a plant or a plant cell or a plant part, e.g., in developing endosperm or a cell or tissue thereof. Accordingly, the present invention provides for use of a promoter, active fragment, derivative, expression construct or expression vector of the present invention for conferring expression on a nucleic acid, e.g., a transgene in a plant cell or plant part, e.g., for conferring expression on a nucleic acid preferentially or selectively in developing endosperm optionally including and/or for repressing or reducing expression of a nucleic acid preferentially or selectively in developing endosperm.

The present invention also provides a method for expressing a nucleic acid in a plant or a plant cell or a plant part, said method comprising:

(i) providing, obtaining or producing a transgenic plant, transgenic plant cell or transgenic plant part comprising a promoter, active fragment, or derivative as described herein according to any embodiment operably connected to a nucleic acid; and
(ii) maintaining said transgenic plant or progeny for a time and under conditions sufficient for said nucleic acid to be expressed.

In one example, the promoter, active fragment or derivative is operably connected to a nucleic acid that is endogenous to the plant cell, plant part or plant. Alternatively, the promoter, active fragment or derivative is operably linked to a transgene, e.g., the transgenic plant, transgenic plant cell or transgenic plant part comprises an expression vector or expression construct of the present invention. Suitable transgenes are described herein and are to be taken to apply mutatis mutandis to the present embodiment of the invention.

In one example, a method for expressing a nucleic acid of the present invention is for conferring expression on the nucleic acid preferentially or selectively in developing endosperm and/or for repressing or reducing expression of the nucleic acid preferentially or selectively in developing endosperm.

Preferably, the method further comprises determining expression or a pattern of expression of the nucleic acid in a plant, plant cell or plant part.

As will be apparent to the skilled artisan based on the foregoing description, by modulating expression of a nucleic acid in a plant cell or plant part a phenotype or trait of a plant cell, plant part, plantlet or whole plant can also be modulated or a phenotype or trait can be conferred on a plant cell, plant part, plantlet or whole plant. Accordingly, the present invention provides for use of a promoter, active fragment, derivative, expression construct or expression vector for modifying a phenotype or trait in a plant cell, plant part, plantlet or whole plant or for conferring a phenotype or trait on a plant cell, plant part, plantlet or whole plant. For example, the plant cell, plant part, plantlet or whole plant has an improved nutritional quality or has a pharmaceutical quality. Alternatively, or in addition the plant part, plantlet or whole plant has modified morphology. Suitable nucleic acids, e.g., transgenes for modulating or conferring one or more traits described herein above are described herein and are to be taken to apply mutatis mutandis to the present embodiment of the invention.

The present invention also provides a method for modulating a phenotype or trait in a plant cell, plant part, plantlet or plant or for conferring a phenotype or trait on a plant cell, plant part, plantlet or plant, said method comprising:

(i) providing, producing or obtaining a plant cell, plant part, plantlet or plant comprising a promoter, active fragment or derivative of the present invention in operable connection with a nucleic acid that when expressed modulates a phenotype or trait in a plant cell, plant part, plantlet or plant or that when expressed confers a phenotype or trait on a plant cell, plant part, plantlet or whole plant; and
(ii) maintaining the plant cell, plant part, plantlet or plant at (i) for a time and under conditions sufficient for the nucleic acid to be expressed and the phenotype or trait to be modified or conferred.

Exemplary traits, phenotypes and nucleic acids are described herein above and are to be taken to apply mutatis mutandis to the present embodiment of the invention.

The present invention also provides a plant cell, plant part, plantlet or plant having a modified phenotype or trait or a new phenotype or trait, said plant cell, plant part, plantlet or plant comprising a promoter, active fragment or derivative of the present invention in operable connection with a nucleic acid that when expressed modulates a phenotype or trait in a plant cell, plant part, plantlet or plant or that when expressed confers a phenotype or trait on a plant cell, plant part, plantlet or whole plant.

Exemplary traits, phenotypes and nucleic acids are described herein above and are to be taken to apply mutatis mutandis to the present embodiment of the invention.

The present inventors have also provided a method for isolating new promoters, e.g., a promoter capable of conferring expression on a nucleic acid in developing endosperm or a cell or tissue thereof. For example, the inventors have provided a method for isolating an endosperm-selective promoter, said method comprising:

(i) identifying an expression product of a gene that is expressed at an increased level in a dormant embryo compared to the level that the expression product is expressed in an imbibed seed or imbibed embryo; and
(ii) isolating a promoter operably connected to said gene wherein said promoter confers expression selectively in endosperm.

Preferably, the method for isolating a promoter as described herein according to any embodiment comprises:

(i) determining the level of expression of a plurality of expression products in a dormant embryo;
(ii) determining the level of expression of a plurality of expression products in an imbibed seed or imbibed embryo;
(iii) identifying one or more expression products expressed at an increased level at (i) compared to (ii); and
(iv) isolating a promoter that confers expression on one or more expression products at (iii).

Preferably, the expression products detected are transcripts or mRNA encoded by a gene. For example, the transcripts or mRNA are detected using a microarray.

This specification contains nucleotide and amino acid sequence information prepared using PatentIn Version 3.5 presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and/or all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

As used herein the term “derived from” shall be taken to indicate that a specified integer may be obtained from a particular source albeit not necessarily directly from that source.

### BRIEF DESCRIPTION OF THE DRAWINGS

FIG. 1a provides graphical representations showing quality of immature embryo total RNA, labelled cRNA and fragmented cRNA samples used for Affymetrix GeneChip® Wheat Genome Arrays.

FIG. 1b provides graphical representations showing quality of 24 hr-imbibed seed total RNA, labelled cRNA and fragmented cRNA samples used for Affymetrix GeneChip® Wheat Genome Arrays.

FIG. 1c provides graphical representations showing quality of 48 hr-imbibed seed total RNA, labelled cRNA and fragmented cRNA samples used for Affymetrix GeneChip® Wheat Genome Arrays.

FIG. 2a is a copy of a photographic representation showing an agarose gel within which nucleic acid fragments from wheat amplified in a GenomeWalker™ assay have been resolved for the isolated of the WP05 promoter sequence. Molecular weight standard has been resolved in lane 6.

FIG. 2b is a copy of a photographic representation showing an agarose gel within which nucleic acid fragments from wheat amplified in a GenomeWalker™ assay have been resolved for the isolated of the WP07 promoter sequence. Molecular weight standard has been resolved in lane 5.

FIG. 3 is a representation of the vector designated pBSubi::bar-nos_R4R3 (SEQ ID NO: 10) which is a base vector for cloning a promoter and/or reporter gene. The vector comprises an Ubi::bar-nos selection cassette and the R4R3 multi-site Gateway™ entry point for promoter, reporter gene and termination sequence Entry Clones. This base vector was used to generate biolistic transformation vectors for each promoter.

FIG. 4 is a representation of the vector pPZP200 35S hph 35S R4R3 (SEQ ID NO: 11) containing the 35S::hph-35St selection cassette and the R4R3 multi-site Gateway™ entry point for promoter, reporter gene and termination sequence Entry Clones. This base vector was used to generate binary transformation vectors for each promoter.

FIG. 5 is a representation of the vector pMPB0098 (SEQ ID NO: 12) which is a binary vector for introducing the WP05 wheat promoter (SEQ ID NO: 3) into cells using Agrobacterium. This vector is derived from pPZP200 35S hph 35S R4R3 into which the wheat promoter, synthetic green fluorescent protein (sGFP) and NOS terminator has been inserted into the R4R3 multi-site Gateway™ entry point.

FIG. 6 is a representation showing the vector pMPB0099 (SEQ ID NO: 13) which is a vector for introduction of the WP05 wheat promoter (SEQ ID NO: 3) into cells using particle bombardment. This vector is derived from pBSubi::bar-nos_R4R3 into which the wheat promoter, synthetic green fluorescent protein (sGFP) and NOS terminator has been inserted into the R4R3 multi-site Gateway™ entry point.

FIG. 7 is a representation of the vector pMPB0084 (SEQ ID NO: 14) which is a binary vector for introducing the 2066 bp wheat promoter from wheat into cells using Agrobacterium. This vector is derived from pPZP200 35S hph 35S R4R3 into which the 2066 bp wheat promoter, synthetic green fluorescent protein (sGFP) and NOS terminator has been inserted into the R4R3 multi-site Gateway™ entry point.

FIG. 8 is a representation showing the vector pMPB0085 (SEQ ID NO: 15) which is a vector for introduction of the 2066 bp wheat promoter from wheat into cells using particle bombardment. This vector is derived from pBSubi::bar-nos_R4R3 into which the 2066 bp wheat promoter, synthetic green fluorescent protein (sGFP) and NOS terminator has been inserted into the R4R3 multi-site Gateway™ entry point.

FIG. 9 is a representation showing the vector pMPB0086 (SEQ ID NO: 16) which is a binary vector for introducing the 2400 bp wheat promoter from wheat into cells using Agrobacterium. This vector is derived from pPZP200 35S hph 35S R4R3 into which the 2400 bp wheat promoter, synthetic green fluorescent protein (sGFP) and NOS terminator has been inserted into the R4R3 multi-site Gateway™ entry point.

FIG. 10 is a representation showing the vector pMPB0087 (SEQ ID NO: 17) which is a vector for introduction of the 2400 bp wheat promoter from wheat into cells using particle bombardment. This vector is derived from pBSubi::bar-nos R4R3 into which the 2400 bp wheat promoter, synthetic green fluorescent protein (sGFP) and NOS terminator has been inserted into the R4R3 multi-site Gateway™ entry point.

FIG. 11 is a representation showing the vector RHF112qc (SEQ ID NO: 18) for expression of the WP05::GUS-nos expression cassette in transgenic maize.

comprising the maize pZMNP-20 promoter operably connected to an intron and a GUS reporter gene.

FIG. 12 is a representation showing the vector RHF121 (SEQ ID NO: 19) for expression of the 2400 bp WP07 promoter in the expression cassette WP07::GUS-nos in transgenic maize.

FIG. 13 is a schematic representation showing the process for used to transform wheat using biolistic transformation.

FIG. 14 provides photographic representations showing the various stages of biolistic transformation of wheat (MPB Bobwhite 26). Panel A shows donor plant production; panels B-D show zygotic embryo isolation and bombardment; panels E-H show callus induction and regeneration under glufosinate selection; panel I shows root formation under selection; panel J shows T0 plants growing under containment glasshouse conditions for recovery of transgenic offspring.

FIG. 15 provides photographic representations showing the various stages of Agrobacterium-mediated transformation of Arabidopsis thaliana using vacuum infiltration. Panel A shows wheat (MPB Bobwhite 26). Panel A shows Arabidopsis thaliana Columbia seeds germinated in small punnets; Panels B and C show approximately 4-week old seedlings used for floral dipping in Agrobacterium suspension under vacuum; Panel D shows Arabidopsis plants isolated and grown to maturity; Panels E and F show seeds surface sterilised and plated on selection media with putative transgenic plants being transferred to soil with an ARACON™ base and tube for T2 seed collection.

FIG. 16 provides photographic representations showing GFP expression driven by the wheat WP05 promoter at 10-14 DAP localized to the endosperm of transgenic seeds but not in embryo or non-transgenic seed.

FIG. 17 provides photographic representations showing GFP expression driven by the wheat WP05 promoter at 25-30 DAP localized to the endosperm of transgenic seeds but not in embryo or non-transgenic seed.

FIG. 18 provides photographic representations showing GFP expression driven by the wheat WP07 promoter at 10-14 DAP localized to the endosperm of transgenic seeds but not in embryo or non-transgenic seed.

FIG. 19 provides photographic representations showing GFP expression driven by the wheat WP07 promoter at 25-30 DAP localized to the endosperm of transgenic seeds but not in embryo or non-transgenic seed.

FIG. 20 provides photographic representations showing strong spatial expression of GUS reporter gene driven by the wheat WP05 promoter in the endosperm of transgenic maize seeds. Expression is visible at 5 DAP in endosperm of transgenic seed.

FIG. 21 provides photographic representations showing strong spatial expression of GUS reporter gene driven by the wheat WP07 promoter in the endosperm of transgenic maize seeds. Expression is visible at 10 DAP in endosperm of transgenic seed.

FIG. 22 provides a schematic representation of a sequence alignment between LOC_Os01 g01290.1 and ZmGSStuc11-12-04.64626.1 obtained from a BLASTn Search of Maize Genomic Assemblies using LOC_Os01g01290.1 as a query sequence with a nucleotide mismatch penalty of −1.

FIG. 23 provides a schematic representation of a sequence alignment between non-overlapping maize gene assemblies ZmGSStuc11-12-04.16895.1 and ZmGSStuc11-12-04.7167.1, obtained from a BLASTn Search of Maize Genomic Assemblies using DQ244863.1 as a query sequence.

FIG. 24 provides a schematic representation of a sequence alignment between DQ244863.1 and the sorghum gene assembly SbGSStuc11-12-04.1189.1, obtained from a BLASTn Search of Sorghum Genomic Assemblies using DQ244863.1 as a query sequence.

### DETAILED DESCRIPTION OF THE PREFERRED EMBODIMENTS

### Sequence Analysis Parameters for Determining a Promoter of the Invention

### a) Sequence Identity Limitations

In determining whether or not two amino acid sequences fall within the defined percentage identity limits herein, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment.

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984). As discussed supra BLAST is also useful for aligning nucleotide sequences and determining percentage identity.

Reference herein to a particular level of sequence identity using the term “at least” or “at least about” shall be taken to encompass any level of sequence identity greater than the recited level. Accordingly, the present invention encompasses a nucleotide sequence or an amino acid sequence at least about 80% identical to a recited sequence, or at least about 85% identical to a recited sequence, or at least about 90% identical to a recited sequence, or at least about 95% identical to a recited sequence, or at least about 98% or 99% identical to a recited sequence.

### b) Analysis of Cis-Acting Elements

Methods for determining whether or not a promoter comprises a cis-acting element will be apparent to the skilled artisan. For example, a promoter is isolated using a method known in the art and/or described herein and the sequence of a promoter is determined using a method known in the art and/or described, for example in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001). For example, a promoter or a fragment thereof of a nucleic acid comprising a sequence encoding a polypeptide comprising at least one minimum GILT domain is isolated using, for example, PCR-based genome walking, or by screening a library of nucleic acids, e.g., as described herein, and the sequence of the promoter determined using, for example, dideoxynucleotide-based sequencing. The sequence is then analysed to determine whether or not it comprises one or more of the cis-acting elements described herein-above.

The sequence of a promoter region may be analysed using suitable software to determine the cis-acting elements contained within that sequence. Suitable software includes:

(i) PLACE (Plant cis-acting DNA elements) as described in Higo et al., Nucl. Acids

Res. 27: 297-300, 1999, and available from National Institute of Agrobiological Sciences, Ibaraki, Japan;

(ii) Plant CARE (cis-acting regulatory elements) Motif Sampler as described in Thijs et al., J Comput Biol. 9: 447-464, 2002 and available from Flanders Interuniversity Institute for Biotechnology (VIB), Zwijnaarde, Belgium; and
(iii) PlantProm database as described in Shahmuradov et al., Nucleic Acids Res. 31:114-7, 2003.

As discussed herein above, the present inventors have identified a plurality of promoters, and by analyzing the sequences of these promoters have identified conserved cis-acting elements, e.g., conserved cis-acting elements from a promoter capable of conferring expression or a pattern of expression on a nucleic acid in a dormant embryo or a cell or tissue thereof. Exemplary cis-acting elements contained in the exemplified promoter sequences are set forth in Tables 4-8 hereof. Exemplary cis-acting elements that are conserved between the five exemplified are set forth in Table 1. Accordingly, it is preferable that a promoter as described herein according to any embodiment comprises one or more of the cis-acting elements set forth in Table 1.

TABLE 1

| Name of element | Sequence | Reference |
|---|---|---|
| ACGTATERD1 (ACGT-related | ACGT | Simpson et al., Plant J., |
| sequence required for etiolation- | | 33: 259-270, 2003 |
| induced expression of erd-1) | | |
| ARR1AT (ARR binding element) | NGATT | Sakai et al., Plant J., 24: |
| | | 703-711, 2000 |
| CACTFTPPCA1 (tetranucleotide | YACT | Gowik et al., Plant Cell, |
| (CACT) from mesophyll expression | | 16: 1077-1090, 2004 |
| module of phosphoenolpyruvate | | |
| carboxylase (PPCA1)) | | |
| CAATBOX1 (CAAT promoter | CAAT | Shirsat, et al., Mol. Gen. |
| consensus sequence) | | Genet., 215: 326-331, |
| | | 1989 |
| CURECORCR (Copper response | GTAC | Quinn et al., J. Biol. |
| element/oxygen response element | | Chem., 275: 6080-6089, |
| from Chlamydomonas) | | 2000 |
| DOFCOREZM (Core site for Dof | AAAG | Yanagisawa and Schmidt, |
| DNA binding) | | Plant J., 17: 209-214, |
| | | 1999 |
| EBOXBNNAPA (E-box napA | CANNTG | Stalberg et al., Planta |
| storage protein gene of Brassica napa | | 199: 515-519, 1996 |
| (R response element) | | |
| GATABOX (GATA Box) | GATA | Lamb and Chua, Plant |
| | | Cell, 1: 1147-1156, 1989 |
| GT1CONSENSUS (consensus GT1 | GRWAAW | Terzaghi and Cashmore |
| binding site) | | Annu. Rev. Plant Physiol. |
| | | Plant Mol. Biol, 46: 445- |
| | | 474, 1995 |
| GTGANTG10 (GTGA motif from | GTGA | Rogers et al., Plant Mol. |
| tobacco late pollen gene g10) | | Biol., 45: 577-585, 2001 |
| IBOXCORE (core sequence from I | GATAA | Terzaghi and Cashmore |
| box conserved in upstream region of | | Annu. Rev. Plant Physiol. |
| light-regulated genes) | | Plant Mol. Biol., 46: 445- |
| | | 474, 1995 |
| MYB2CONSENSUSAT | YACKG | Abe et al., Plant Cell 15: |
| (MYB recognition site | | 63-78 2003 |
| found in the promoters of | | |
| dehydration-responsive | | |
| gene rd22) | | |
| MYBCORE (core sequence | CNGTTR | Urao et al., Plant Cell |
| of binding site of MYB | | 5: 1529-1539 1993 |
| proteins) | | |
| MYBCOREATCYCB1 | AACGG | Planchais et al., Plant |
| (core sequence of binding | | Mol. Biol., 50: 111-127, |
| site for MYB from | | 2002 |
| Arabidopsis cyclin B1 gene) | | |
| MYBST1 (Core motif of a | GGATA | Baranowskij et al., |
| potato MYB homolog | | EMBO J 13: 5383-5392 |
| binding site) | | 1994 |
| MYCCONSENSASAT | CANNTG | Abe et al., Plant Cell 15: |
| (MYC recognition sequence | | 63-78 2003 |
| from dehydration- | | |
| responsive gene rd22) | | |
| PRECONSCRHSP70A | SCGAYNRNNNNNNNNNNNNNNNHD | Von Grommoff et al., |
| (consensus sequence of | | Nucl. Acids Res., 34: |
| plastid response element in | | 4767-4779, 2006 |
| promoter of HSP70 in | | |
| Chlamydomonas) | | |
| WRKY71OS (Core of | TGAC | Zhang et al., Plant |
| TGAC-containing W box | | Physiol., 134: 1500-1513, |
| from Amy32b promoter) | | 2004 |

It is to be understood that the precise number of any specific cis-acting element in a promoter of the present invention may vary according to length and additional elements to those specifically indicated in Table 1 are permissible. A skilled artisan can readily ascertain any number of variations to the elements presented in Table 1 from the data provided herein e.g., in Tables 4-8.

### Plant Source of a Promoter of the Invention

In one example, a promoter as described herein according to any embodiment is from wheat e.g., SEQ ID Nos: 3-5 hereof or comprising the repertoire of cis-acting elements presented in Table 4 and/or Table 5 or a repertoire of cis-acting elements conserved between those presented in Table 4 and Table 5 without necessary regard to their precise orientation and/or positioning in each individual sequence.

The term “wheat” is to be taken in its broadest context to mean an annual or biennial grass capable of producing erect flower spikes and light brown grains and belonging to the Aegilops-Triticum group including Triticum sp. and Aegilops sp. The term “wheat” thus extends to any of various annual cereal grasses of the genus Triticum such as those that are generally cultivated in temperate regions for their edible grain used to produce flour e.g., for use in breadstuffs and/or biscuits and/or noodles and/or pasta. Suitable species and/or cultivars will be apparent to the skilled artisan based on the description herein.

The term “wheat” also includes any tetraploid, hexaploid and allopolyploid (e.g., allotetraploid and allohexaploid) Aegilops sp. or Triticum sp. which carries the A genome and/or the B genome and/or D genome of the allohexaploid Triticum aestivum or a variant thereof. This includes A genome diploids (e.g., T. monococcum and T. urartu), B genome diploids (e.g., Aegilops speltoides and T. searsii) and closely-related S genome diploids (e.g., Aegilops sharonensis), D genome diploids (e.g., T. tauschii and Aegilops squarrosa), tetraploids (e.g., T. turgidum and T. dicoccum (AABB), Aegilops tauschii (AADD)), and hexaploids (e.g., T. aestivum and T. compactum). The term “wheat” may encompass varieties, cultivars and lines of Aegilops sp. or Triticum sp. but is not to be limited to any specific variety, cultivar or line thereof unless specifically stated otherwise.

Preferably, the wheat is T. aestivum or T. turgidum (formerly known as T. durum) or a variety, cultivar or line thereof, optionally selected for a seed quality trait e.g., yield, bread-making quality, biscuit-making quality, or noodle/pasta-making quality.

As will be apparent to the skilled artisan from the foregoing, many varieties of wheat are polyploid. Accordingly, any single wheat genome may comprise a plurality of promoters as defined herein to be part of the invention. The present invention clearly contemplates any and/or all of those promoters.

In another example of the invention, a promoter as described herein according to any embodiment is from maize e.g., SEQ ID Nos: 7 and 8 hereof or comprising the repertoire of cis-acting elements presented in Table 6 and/or Table 8 or a repertoire of cis-acting elements conserved between those presented in Table 6 and Table 8 without necessary regard to their precise orientation and/or positioning in each individual sequence. The term “maize” shall be taken to mean grass of the genus Zea. Preferably, the term maize encompasses any plant of the species Zea mays. The term maize includes such species as, for example, Z. mays indurata, Z. mays indenta, Z. mays everta, Z. mays saccharata, Z. mays amylacea, Z. mays tunicata and/or Z. mays Ceratina Kulesh.

In another example of the invention, a promoter as described herein according to any embodiment is from rice e.g., SEQ ID No: 6 hereof or comprising the repertoire of cis-acting elements presented in Table 5 without necessary regard to their precise orientation and/or positioning in each individual sequence. The term “rice” shall be taken to mean grass of the genus Oryza, including indica and japonica rice species and varieties. Preferably, the term rice encompasses any plant of the species Oryza sativa.

In further examples, a promoter as described herein according to any embodiment is from barley or sorghum or rye or millet (e.g. pearl millet or proso millet) or buckwheat (e.g., of the family Polygonaceae) or oat (e.g., Avena sativa) or a cell, tissue or organs from any other plant from the family Graminaceae, Gramineae or Poaceae.

### Isolation of Promoters

A promoter as described herein according to any embodiment is isolated using any of a variety of molecular biology techniques. For example, a promoter is isolated using polymerase chain reaction using primers based on the sequence of a promoter described herein, e.g., in any one or more of SEQ ID NOs: 3-9. For example, a pair of primers comprising at least about 20 to about 30 nucleotides that is capable of hybridizing to a nucleic acid comprising a sequence set forth in any one or more of SEQ ID NOs: 3-9 is produced. Preferably, one or both of the primers is capable of hybridizing to a plurality of sequences set forth in SEQ ID NOs: 3-9, i.e., the primers hybridize to a conserved region and/or are degenerate. Suitable methods for designing and producing primers for PCR are known in the art and/or described in Dieffenbach (ed) and Dveksler (ed) (In: PCR Primer: A Laboratory Manual, Cold Spring Harbour Laboratories, NY, 1995). These primers are then hybridized to different strands of a nucleic acid template, e.g., genomic DNA from a plant, and specific nucleic acid copies of the template are amplified enzymatically. Following amplification, the amplified nucleic acid is isolated using a method known in the art and, preferably cloned into a suitable vector. Such a method is useful for isolating a promoter from nucleic acid, preferably genomic DNA from any plant.

Alternatively, or in addition, an oligonucleotide is produced that is capable of hybridizing to a promoter described herein according to any embodiment. Preferably, the oligonucleotide is capable of hybridizing to a region of a promoter as described herein according to any embodiment that is conserved in a plurality of promoters.

Alternatively, or in addition, the oligonucleotide is capable of hybridizing to a plurality of promoters as described herein according to any embodiment under low or moderate stringency conditions. Such an oligonucleotide is then used to screen a nucleic acid library, e.g., a library comprising fragments of genomic DNA from a plant using a method known in the art and described, for example, in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001). A suitable fragment is then isolated and, if necessary, the promoter isolated from the fragment.

A suitable promoter may also be isolated based on its ability to confer expression in developing endosperm. For example, using one or more oligonucleotide primers that hybridize to a promoter of the invention RT-PCR is performed using mRNA from a developing endosperm to amplify a fragment of a cDNA comprising such a nucleic acid. This fragment is then used to isolate a promoter that confers expression or a pattern of expression on said mRNA. For example, as described herein, genome-walking is used to isolate a promoter. In such a method, genomic DNA from a plant is cleaved, e.g., using a restriction endonuclease and subsequently ligated to an adaptor having a known sequence. PCR is then performed using a primer capable of annealing to the adaptor and a primer capable of annealing to the fragment of cDNA. In this manner, sequence upstream or 5′ to the sequence linked to the promoter in its native context is isolated, including the promoter sequence.

Alternatively, an oligonucleotide is used to screen a genomic DNA library from a plant to isolate a fragment of genomic DNA comprising a gene or fragment thereof comprising the promoter. Sequence from the isolated genomic DNA fragment may then be used to isolate additional genomic DNA fragments. By analyzing the nucleotide sequence of the genomic DNA, e.g., using a method described herein, the sequence of a promoter is determined.

In-silico screening is also useful for identifying a suitable promoter. For example, the inventors have identified a number of conserved regions of a gene to which a promoter as described herein according to any embodiment is operably connected in nature. Based on one or more of these sequences, a database of sequences from a plant, e.g., a database comprising genomic DNA sequences is searched, and sequences homologous to the conserved region(s) identified. Sequence upstream of the identified region is then analysed to identify the sequence of a promoter operably connected thereto. In silico methods of promoter prediction are known in the art and described, for example, in Shahmuradov et al., Nucleic Acids Research 33:1069-1076, 2005, or using plant promoter prediction software available from the School of Biological Sciences, Royal Holloway University of London.

A promoter identified using any of the methods described supra should be tested empirically to determine its ability to confer expression on a nucleic acid, e.g., in a developing endosperm or a cell or tissue thereof. Suitable methods for testing a promoter will be apparent to the skilled artisan based on the description herein.

### Ability of a Promoter, Active Fragment or Derivative to Confer Endosperm Expression

Methods for determining the ability of a promoter or a fragment thereof or a derivative thereof to confer expression on nucleic acid include, for example, determining the ability of the promoter, fragment, derivative to induce expression of a reporter gene in a cell, tissue or organ of a plant.

For example, a promoter or a fragment or a derivative as described herein according to any embodiment is placed in operable connection with a reporter gene, e.g., a reporter gene that produces a detectable signal or a reporter gene that permits selection of a cell expressing the gene.

Reporter genes will be apparent to the skilled artisan and include, for example, a bar gene (bialaphos resistance gene), a bacterial neomycin phosphotransferase II (nptII) gene, a hygromycin phosphotransferase gene, an aacC3 gene, an aacC4 gene, a chloramphenicol acetyl transferase gene, a gene encoding 5-enolpyruvyl-shikimate-3-phosphate synthase or a gene encoding phosphinothricin synthase. Each of these genes confers resistance to a herbicide or an antibiotic. Alternatively, the reporter gene confers the ability to survive and/or grow in the presence of a compound in which an untransformed plant cell cannot grow and/or survive, e.g., a mana gene (Hansen and Wright, Trends in Plant Sciences, 4: 226-231, 1999), a cyanamide hydratase (Cah) gene (SEQ ID NO: 26) (as described in U.S. Ser. No. 09/518,988) or a D-amino oxidase, (DAAO) gene (Erikson et al., Nature Biotechnology, 22: 455-458, 2004).

Reporter genes that produce a detectable expression product when expressed include, for example, a β-glucuronidase gene (GUS; the expression of which is detected by the metabolism of 5-bromo-4-chloro-3-indolyl-1-glucuronide to produce a blue precipitate), a bacterial luciferase gene, a firefly luciferase gene (detectable following contacting a plant cell with luciferin), or a fluorescent reporter gene, e.g., monomeric discosoma red fluorescent protein (Campbell et al., Proc Natl Acad Sci USA. 99:7877-7882, 1992) or a monomeric GFP from Aequorea coerulescens (Gurskaya et al., Biochem J. 373:403-408, 2003).

Following linkage of a promoter or fragment, or derivative as described herein according to any embodiment to a suitable reporter gene, the resulting expression construct is transformed into a plant cell or plant part or plant, e.g., using a method as described herein. Expression of the reporter gene is then detected. For example, in the case of a selectable reporter gene, transformed plant cell, parts or plants are grown in the presence of a suitable herbicide or antibiotic, and only those embryos or cells expressing the reporter gene are capable of growing. In the case of a detectable reporter gene, a plant cell, plant part or whole plant is analysed to detect expression of the detectable reporter gene expression product, e.g., fluorescence or metabolism of a substrate to produce a detectable metabolite.

Alternatively, a plant cell or tissue is transformed using a method known in the art and/or described herein. The transformed cell or tissue is then used to regenerate a plant. Alternatively, the plant is bred, and offspring of the plant grown. This process provides an additional advantage in so far as it permits the level of expression of a reporter gene to be detected in a variety of tissues and at various developmental stages. In the case of identifying a promoter that confers expression of a nucleic acid in a developing endosperm, plants are grown until they produce seeds. Endosperm from the dormant seeds is then analysed to detect expression of a reporter gene Such a method permits the identification of promoters that preferentially or selectively express a reporter gene in a developing endosperm or a cell or tissue thereof.

The ability of a promoter to confer expression or a pattern of expression on a nucleic acid, e.g., in a developing endosperm or a cell or tissue may also be determined by determining the expression pattern of an expression product of a nucleic acid linked to the promoter in nature, for example, using Northern blotting, quantitative PCR, microarray analysis or an immunoassay. Suitable methods will be apparent to the skilled artisan and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

For example, as exemplified herein the present inventors have performed microarray analysis to detect the level of expression of a nucleic acid linked to a promoter as described herein according to any embodiment in various tissues. This process involves isolating mRNA from a variety of tissues from a plant, producing copy RNA (cRNA) and labelling the cRNA, e.g., using a fluorescent label such as Cy5. Copy RNA from a control tissue is also labelled with a different label to that used to label the test cRNA, e.g., Cy5, and the two samples mixed. The labelled cRNA is then contacted with a solid substrate having immobilized thereon an oligonucleotide capable of specifically hybridizing to a nucleic acid linked to the promoter of interest. Following a sufficient time for the labelled mRNA to hybridize to the oligonucleotide, the solid substrate is washed and the level of fluorescence of each label detected. In this manner the level of expression of the nucleic acid of interest in a test sample is determined relative to the level in a control sample. Using such a method, the present inventors showed that a transcript encoded by a gene operably connected to a promoter as described herein according to any embodiment is expressed at an increased level in a developing endosperm (test sample) relative to a mature seed, vegetative tissue or reproductive tissue in which an exemplified promoter of the invention does not confer significant expression (control sample).

The present inventors have also used quantitative RT-PCR to determine the level of expression of a nucleic acid linked to a promoter as described herein according to any embodiment. Suitable methods for performing such quantitative RT-PCR will be apparent to the skilled artisan and/or described for example, U.S. Pat. No. 6,174,670.

### Active Promoter Fragments

The present invention also encompasses a fragment of a promoter described herein according to any embodiment. In one example, such an active fragment retains the ability of the promoter to confer expression or a pattern of expression on a nucleic acid in a developing endosperm or a cell or tissue thereof. In this respect, the fragment need not confer the same level of expression or pattern of expression as a promoter from which it is derived. For example, the fragment induces expression of a nucleic acid to which it is operably connected to a lesser degree than a promoter from which it is derived, e.g., because it lacks a binding site for a transcription factor. Alternatively, a fragment may induce expression of a nucleic acid to which it is operably connected to a greater degree than a promoter from which it is derived, e.g., because it lacks a binding site for a protein that suppresses transcription.

In one example, the present invention provides an active fragment of a promoter as described herein according to any embodiment, said active fragment comprising at least about 200 base pairs (bp) or at least about 500 bp or at least about 700 bp or at least about 900 bp or at least about 1000 bp e.g., derived from an exemplified promoter set forth in the Sequence Listing.

In another example, an active promoter fragment of the present invention at least comprises a basal promoter regulatory region from a full-length promoter, such as a minimal sequence necessary and/or sufficient for transcription initiation in seed endosperm. A basal promoter regulatory region comprises a functional TATA box element e.g., positioned between about 15 and about 50 nucleotides upstream from the site of transcription initiation, and preferably between about 15 and about 40 nucleotides upstream from the site of transcription initiation, and more preferably between about 15 and about 30 or 35 nucleotides upstream from the site of transcription initiation. For the purposes of nomenclature, a basal promoter regulatory region in this context comprises the terminal 100 or 90 or 80 or 70 or 60 or 50 or 40 nucleotides of any one of SEQ ID Nos: 3-9 or a sequence complementary thereto.

Preferred basal promoter regulatory regions also comprise a CCAAT box element (e.g., the sequence CCAAT or GGGCG) positioned between about 40 and about 200 nucleotides or between about 50 and about 150 nucleotides or between about 60 and about 120 nucleotides upstream from the transcription start site. For the purposes of nomenclature, a basal promoter regulatory region in this context comprises the terminal 200 or 190 or 180 or 170 or 160 or 150 or 140 or 130 or 120 or 110 or 100 or 90 or 80 or 70 or 60 or 50 nucleotides of any one of SEQ ID Nos: 3-9 or a sequence complementary thereto.

Active fragments that comprise a basal promoter regulatory region and one or more upstream elements of the native promoter are also provided by the present invention. For example, active fragments may comprise the terminal 500 nucleotides, or the terminal 400 nucleotides or the terminal 300 nucleotides or the terminal 200 nucleotides of any one of SEQ ID Nos: 3-9 or a sequence complementary thereto.

Alternatively, such active fragments may be truncated at their 3′-ends compared to the promoter sequences set forth in any one of SEQ ID Nos: 3-9, e.g., by deletion of sequences downstream of the transcriptional start site. For example, active fragments may comprise a sequence from about 500 nucleotides to about 40 nucleotides upstream of the 3′-end of any one of SEQ ID Nos: 3-9 or complementary thereto, or from about 400 nucleotides to about 40 nucleotides upstream of the 3′-end of any one of SEQ ID Nos: 3-9 or complementary thereto, or from about 300 nucleotides to about 40 nucleotides upstream of the 3-end of any one of SEQ ID Nos: 3-9 or complementary thereto, or from about 200 nucleotides to about 40 nucleotides upstream of the 3′-end of any one of SEQ ID Nos: 3-9 or complementary thereto, or from about 400 nucleotides to about 50 nucleotides upstream of the 3′-end of any one of SEQ ID Nos: 3-9 or complementary thereto, or from about 500 nucleotides to about 60 nucleotides upstream of the 3′-end of any one of SEQ ID Nos: 3-9 or complementary thereto, or from about 300 nucleotides to about 70 nucleotides upstream of the 3′-end of any one of SEQ ID Nos: 3-9 or complementary thereto, or from about 200 nucleotides to about 80 nucleotides upstream of the 3′-end of any one of SEQ ID Nos: 3-9 or complementary thereto. Other fragments are not to be excluded. Such active fragments preferably comprise one or more conserved sequence motifs as disclosed herein.

Suitable methods for producing a fragment of a promoter as described herein according to any embodiment will be apparent to the skilled artisan and/or described for example in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001). For example, a previously isolated promoter is cleaved using any known method, e.g., using one or more restriction endonucleases and the resulting fragment(s) are then assayed to determine their ability to confer expression or a pattern of expression on a nucleic acid in developing endosperm or cell or tissue thereof. Alternatively, a fragment of a promoter as described herein according to any embodiment is amplified using a nucleic acid amplification reaction, e.g., PCR. The resulting fragment is then assayed to determine whether or not it is capable of conferring expression or a pattern of expression on a nucleic acid, e.g., in developing endosperm.

Suitable methods for determining the ability of a fragment to confer expression or a pattern of expression on a nucleic acid are described herein.

### Promoter Derivatives

Promoter derivatives encompassed by the present invention include a promoter derived from a promoter as described herein according to any embodiment, however comprising one or more additional regulatory elements, derived from either an exemplified promoter or a heterologous promoter. For example, such an additional regulatory element further enhances expression of a nucleic acid to which it is operably connected and/or alters the timing of expression of a sequence to which it is operably connected. For example, such a chimeric promoter that comprise the nucleotide sequence set forth in SEQ ID NO: 3, 4, 5, 6, 7, 8 or 9 may be modified by the inclusion of nucleic acid from a different endosperm-operable promoter to further enhance expression of a nucleic acid to which the promoter is operably connected in developing endosperm or a cell or tissue thereof. The performance of such embodiments is readily achievable by those skilled in the art.

Those skilled in the art will be aware that it is also possible to modify the level of structural gene expression and/or the timing of structural gene expression and/or the location of structural gene expression in a plant or plant part by mutation of a regulatory genetic sequence (e.g., cis-acting element or 5′-non-coding region, etc) within the promoter sequence to which a nucleic acid is operably connected. For example, to achieve such an objective, the promoter sequence of the present invention is subjected to mutagenesis to produce single or multiple nucleotide substitutions, deletions and/or additions.

Alternatively, or in addition, the arrangement of specific regulatory sequences within the promoter may be altered, including the deletion therefrom of certain regulatory sequences and/or the addition thereto of regulatory sequences derived from the same or a different promoter sequence.

Preferred derivatives of a promoter as described herein according to any embodiment comprise one or more functional cis-acting elements present in a promoter as described herein according to any embodiment, for example, a cis-acting element required for or associated with conferring expression or a pattern of expression.

Derivatives of the promoter can be produced by synthetic means or alternatively, derived from naturally-occurring sources.

For example, the promoter sequence may be derivatized without complete loss of function such that it at least comprises one or more of the following sequences:

(i) a 5′-non-coding region; and/or
(ii) one or more cis-regulatory regions, such as one or more functional binding sites for a transcriptional regulatory proteins or translational regulatory proteins, one or more upstream activator sequences, enhancer elements or silencer elements; and/or
(iii) a TATA box motif; and/or
(iv) a CCAAT box motif; and/or
(v) an upstream open reading frame (uORF); and/or
(vi) a transcriptional start site; and/or
(vii) a translational start site; and/or
(viii) a nucleotide sequence which encodes a leader sequence.

As used herein, the term “5′ non-coding region” shall be taken in its broadest context to include all nucleotide sequences which are derived from the upstream region of a gene, e.g., a gene expressed in developing endosperm, other than those sequences which encode amino acid residues comprising the polypeptide product of said gene. Such regions include an intron, e.g., an intron derived from a ubiquitin gene.

As used herein, the term “uORF” refers to a nucleotide sequence localised upstream of a functional translation start site in a gene and generally within the 5′-transcribed region (i.e. leader sequence), which encodes an amino acid sequence. Whilst not being bound by any theory or mode of action, a uORF functions to prevent over-expression of a structural gene sequence to which it is operably connected or alternatively, to reduce or prevent such expression.

Other derivative promoters contemplated by the present invention include, for example, a bi-directional promoter comprising a promoter as described herein according to any embodiment. Such a bi-directional promoter comprises, for example, (i) a promoter as described herein according to any embodiment and positioned to confer expression or a pattern of expression on a nucleic acid linked to, e.g., the 3′ end thereof; and (ii) a second promoter linked to the 5′ end of the promoter at (i) and positioned to confer expression or a pattern of expression on a nucleic acid linked to the 5′ end of the second promoter. Clearly, the second promoter may also be a promoter as described herein according to any embodiment.

### Expression Constructs and Expression Vectors

Following isolation of a promoter as described herein according to any embodiment, an expression construct may be produced. Such an expression construct comprises a promoter, active fragment or derivative as described herein according to any embodiment operably connected to a nucleic acid to be expressed, i.e., a transgene, e.g., a nucleic acid encoding a polypeptide of interest, or a nucleic acid that is transcribed to encode, e.g., a siRNA, ribozyme, microRNA or RNAi.

The present invention contemplates linking a promoter, active fragment or derivative as described herein according to any embodiment to any transgene. Suitable examples of transgenes will be apparent to the skilled artisan and/or described herein.

Methods for linking a promoter, active fragment or derivative as described herein according to any embodiment and a transgene will be apparent to the skilled artisan and include, for example, ligating the promoter, active fragment or derivative to the transgene, e.g., using T4 DNA ligase. Alternatively, or in addition a fusion of the promoter, active fragment or derivative and transgene is produced using recombinant means, e.g., splice-overlap extension. Suitable methods for linking two or more nucleic acids are also described in, for example, Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001).

Such an expression construct may comprise additional components, such as, for example, a sequence encoding a targeting sequence or a detectable label. Such an additional component may be located between the promoter and the transgene, e.g., such that it is expressed as a 5′ fusion with the polypeptide encoded by the transgene. Alternatively, the additional component may be located 3′ to the transgene.

A targeting sequence is a sequence of amino acids within a polypeptide that directs the polypeptide to a particular subcellular location. Targeting sequences useful for the performance of the invention are known in the art and described in, for example, Johnson et al., The Plant Cell 2:525-532, 1990; Mueckler et al. Science 229:941-945, 1985; Iturriaga et al. The Plant Cell 1:381-390, 1989; McKnight et al., Nucl. Acid Res. 18:4939-4943, 1990; Matsuoka and Nakamura, Proc. Natl. Acad. Sci. USA 88:834-838, 1991. Furthermore, the book entitled “Recombinant proteins from plants”, Eds. C. Cunningham and A. J. R. Porter, 1998 Humana Press Totowa, N.J. describe various suitable methods for the production of recombinant proteins in plants and methods for targeting the proteins to different compartments in the plant cell.

Suitable detectable markers include, for example, an epitope, e.g., influenza virus hemagglutinin (HA), Simian Virus 5 (V5), polyhistidine, c-myc, FLAG.

Alternatively, or in addition, a promoter, active fragment or derivative as described herein according to any embodiment is included in an expression vector. In this respect, such an expression vector may comprise a transgene operably connected to a promoter, active fragment or derivative as described herein according to any embodiment. Alternatively, or in addition, an expression vector may comprise a means for inserting a transgene such that it is in operable connection with the promoter, fragment or derivative. Such means include, for example, a multiple cloning site comprising one or more restriction endonuclease cleavage site(s). Additional means include one or more recombination site(s).

Additional components of an expression vector will be apparent to the skilled artisan and include, for example, an origin of replication, e.g., to permit replication of the vector in a bacterial cell, e.g., a ColE1 origin of replication.

An expression vector may also comprise a selectable marker, e.g., as described supra, operably connected to a promoter. For example, the selectable marker may be operably connected to a ubiquitous promoter, such as a promoter from ubiquitin (ubi) or from the cauliflower mosaic virus, e.g., CaMV 35S. Suitable promoters and selectable markers will be apparent to the skilled artisan.

In the case of an expression vector to be delivered into a plant using Agrobacterium-based transformation, the vector preferably comprises a left-border (LB) sequence and a right-border (RB) sequence that flank the transgene to be delivered into the plant cell, i.e., the transfer DNA. Such a vector may also comprise a suitable selectable marker for selection of bacteria comprising the vector, e.g., conferring resistance to ampicillin.

Preferably, the vector is a binary Ti plasmid or Ri plasmid. Binary Ti plasmids or Ri plasmids are produced based on the observation that the T-DNA (nucleic acid transferred to a plant cell) and the vir genes required for transferring the T-DNA may reside on separate plasmids (Hoekema et al., Nature, 303: 179-180, 1983). In this respect, the vir function is generally provided by a disarmed Ti plasmid resident in or endogenous to the Agrobacterium strain used to transform a plant cell.

Accordingly, a binary Ti plasmid or Ri plasmid comprises a transgene located within transfer-nucleic acid (e.g., T-DNA). Such transfer-nucleic acid comprising the transgene is generally flanked by or delineated by a LB and a RB.

Suitable binary plasmids are known in the art and/or commercially available. For example, a selection of binary Ti vectors includes pBIN19 (Bevan et al., Nucleic Acids Res., 12: 8711-8721, 1984); pC22 (Simoens et al., Nucleic Acids Res. 14: 8073-8090, 1986); pGA482 (An et al., EMBO J. 4: 277-284, 1985); pPCV001 (Koncz and Schell Mol. Gen. Genet. 204: 383-396, 1986); pCGN1547 (McBride and Summerfelt 14: 269-276, 1990); pJJ1881 (Jones et al., Transgenic Res. 1: 285-297, 1992); pPZP111 (Hajukiewicz et al., Plant Mol. Biol., 25: 989-994, 1994); and pGreen0029 (Hellens et al., Plant Mol. Biol., 42: 819-832, 2000).

Additional binary vectors are described in, for example, Hellens and Mullineaux Trends in Plant Science 5: 446-451, 2000. Variants of these plasmids e.g., as described herein or known in the art may also be employed.

Suitable Ri plasmids are also known in the art and include, for example, pRiA4b (Juouanin Plasmid, 12: 91-102, 1984), pRi1724 (Moriguchi et al., J. Mol. Biol. 307:771-784, 2001), pRi2659 (Weller et al., Plant Pathol. 49:43-50, 2000) or pRi1855 (O'Connell et al., Plasmid 18:156-163, 1987).

### Transgenes

As discussed supra, the present invention encompasses an expression construct or expression vector comprising a promoter, active fragment or derivative as described herein according to any embodiment linked to any transgene.

In one example, a transgene encodes a polypeptide that is to be expressed in developing endosperm or cell or tissue thereof of a plant. For example, the transgene encodes a polypeptide that is involved in biosynthesis of starch or storage protein. Expression of such a transgene is useful for prolonging grain filling or enhancing yield characteristics, or to enhance a nutritional quality of seed. Such an expression construct is useful for, for example, improving end-product traits, and includes, without limitation, those encoding seed storage proteins, fatty acid pathway enzymes, tocopherol biosynthetic enzymes, amino acid biosynthetic enzymes, and starch branching enzymes. For example, a suitable seed storage protein includes a zein (e.g., as described in U.S. Pat. Nos. 4,886,878, 4,885,357 and 5,215,912), 7S proteins (e.g., as described in U.S. Pat. Nos. 5,003,045, and 5,576,203), a brazil nut protein (e.g., as described in U.S. Pat. No. 5,850,024), a phenylalanine free protein (e.g., as described in PCT Publication WO 96/17064), albumin (e.g., as described in PCT Publication WO 97/35023).

Examples of fatty acid pathway enzymes include, for example, a thioesterase (e.g., as described in U.S. Pat. Nos. 5,512,482, 5,530,186 and 5,945,585), and a desaturase (e.g., as described in U.S. Pat. Nos. 5,689,050, 5,663,068 and 5,614,393). In one example, expression of a stearoyl-ACP desaturase-encoding gene is down-regulated to thereby increase stearic acid content of the seed e.g., Knultzon, et al., Proc. Natl. Acad. Sci. USA 89, 2624 (1992) and WO99/64579. In another example, oleic acid content is elevated or enhanced via FAD-2 gene modification and/or by decreasing linolenic acid content via FAD-3 gene modification e.g., U.S. Pat. Nos. 6,063,947; 6,323,392; and 6,372,965; and WO 93/11245. In another example, the content of conjugated linolenic or linoleic acid content is modified e.g., WO 01/12800. In another example, the expression of one or more genes selected from LEC1, AGP, Dek1, Superal1, mi1ps and lpa genes (e.g., lpa1, lpa3, hpt or hggt) is modified e.g., WO 02/42424, WO 98/22604, WO 03/011015, U.S. Pat. No. 6,423,886, U.S. Pat. No. 6,197,561, U.S. Pat. No. 6,825,397, US Patent Publication Nos. 20030079247, 20030204870, and WO 02/057439 and WO 03/011015, and Rivera-Madrid, et. al., Proc. Natl. Acad. Sci. 92, 5620-5624, 1995.

In another example to achieve a particularly high content of polyunsaturated fatty acid (PUFA; e.g., C₁₈—, C₂₀- or C₂₂-fatty acids having at least two or three or four or five or six double bonds) in transgenic plants, one or more PUFA biosynthesis genes is expressed under control of a promoter, active fragment or derivative of the present invention. Optionally, a plurality of such genes is expressed separately under the control of a plurality of promoters, active fragments or derivatives thereof, wherein at least one promoter, active fragment or derivative is a promoter, active fragment or derivative of the present invention, and one or more other promoters active in embryo and/or endosperm is employed in a gene stacking approach. For example, PUFA content is enhanced by altering expression of a polypeptide having acyl-CoA:lysophospholipid acyltransferase activity, e.g., wherein the acyl-CoA:lysophospholipid acyltransferases encoded by the nucleic acid sequence specifically convert C₁₆-, C₁₈-, C₂₀- or C₂₂-fatty acids, and optionally altering expression of one or more acyl-CoA dehydrogenase(s) and/or one or more acyl-ACP [=acyl carrier protein] desaturase(s) and/or one or more acyl-ACP thioesterase(s) and/or one or more fatty acid acyl transferase(s) and/or one or more fatty acid synthase(s) and/or one or more fatty acid hydroxylase(s) and/or one or more acetyl-coenzyme A carboxylase(s) and/or one or more acyl-coenzyme A oxidase(s) and/or one or more fatty acid desaturase(s) and/or one or more fatty acid acetylenases and/or one or more lipoxygenases and/or one or more triacylglycerol lipases and/or one or more allenoxide synthases and/or one or more hydroperoxide lyases and/or one or more fatty acid elongase(s). Particularly preferred transgenes to be expressed under control of a promoter of the present invention or an active fragment or derivative thereof include, for example, one or more M-desaturases and/or one or more Δ5-desaturases and/or one or more Δ6-desaturases and/or one or more Δ8-desaturases and/or one or more Δ9-desaturases and/or one or more Δ12-desaturases and/or one or more Δ5-elongases and/or one or more Δ6-elongases and/or one or more Δ9-elongases (US Pat. Pub. No. 20090094707). In such examples involving gene stacking, only one of the introduced transgenes e.g., a M-desaturase or Δ5-desaturases or Δ6-desaturase or Δ8-desaturase or Δ9-desaturase or Δ12-desaturase or Δ5-elongase or M-elongase or Δ9-elongase, need be placed operably under control of a promoter of the present invention in the sense or antisense orientation. Transgenic plants which contain the polyunsaturated fatty acids synthesized in the process according to the invention are marketed directly without there being any need for the oils, lipids or fatty acids synthesized to be isolated. Harvested material, plant tissue, reproductive tissue and cell cultures which are derived from the transgenic plant may also be used. Products of the transgenic plants according to the invention can also be isolated in the form of oils, fats, lipids and/or free fatty acids. Polyunsaturated fatty acids produced by this process can be obtained by harvesting the organisms, either from the crop in which they grow, or from the field e.g., by pressing or other extraction process such as cold-beating or cold-pressing or pre-treating seeds by comminution, steam or roasting and solvent-based extraction e.g., using warm hexane. Thereafter, the resulting products are processed further, i.e. refined to remove plant mucilage and suspended matter, desliming, and base extraction of fatty acids e.g., using sodium hydroxide, drying, bleaching, and deodorizing.

In another example, phosphorus content of the endosperm is modified by expressing a phytase-encoding gene under the control of a promoter, active fragment or derivative thereof in the endosperm to thereby enhance breakdown of phytate and increase the availability of free phosphate to the transformed plant. An Aspergillus niger phytase gene is disclosed e.g., by Van Hartingsveldt et al., Gene 127:87 (1993).

In another example, a gene that reduces phytate content is expressed operably under the control of a promoter or active fragment or derivative thereof according to the present invention. In maize, this is accomplished by expressing an LPA allele (e.g., Raboy et al., (1990) Maydica 35:383) and/or by altering inositol kinase activity (e.g., WO 02/059324, US Patent Publication No. 20030009011, WO 03/027243, US Pat. Publication No. 20030079247, WO 99/05298, U.S. Pat. No. 6,197,561, US. Pat. No. 6,291,224, U.S. Pat. No. 6,391,348, WO2002/059324, US Patent Publication No. 2003/0079247, WO 98/45448, WO 99/55882, WO 01/04147).

In yet another example, a promoter of the present invention or an active fragment or derivative thereof is employed to express a nutritional protein such as a phytase. Grain from graminaceous plants is also widely used as an animal feed for non-ruminant animals and phytase of Aspergillus niger is used as a supplement in animal feeds to improve the digestibility and also improve the bioavailability of phosphate and minerals. In one example, a promoter, active fragment or derivative as described herein according to any embodiment is used to express the phyA gene from A. niger in the developing endosperm.

In another example, the promoter, active fragment or derivative of the present invention is utilized to modify tocotrienol and/or tocopherol content. Tocotrienols are vitamin E-related compounds whose occurrence in plants is limited primarily to the seeds of monocots e.g., palm, wheat, rice and barley. Tocotrienols are structurally similar to tocopherols, including alpha-tocopherol which is a form of vitamin E. Tocopherols and tocotrienols are potent lipid-soluble antioxidants having considerable nutritive value in human and animal diets e.g., Packer et al. J. Nutr. 131:369 S-373S (2001), and as cholesterol lowering compounds e.g., Theriault et al. Clin. Biochem. 32, 309-319, 1999; Qureshii et al. J. Biol. Chem. 261, 10544-10550, 1986. By expressing 2-methyl-6-phytylbenzoquinol methyltransferase (VTE3) and/or tocopherol cyclase (VTE1) and/or gamma-tocopherol methyltransferase (VTE4) operably under control of a promoter of the present invention, the levels of one or more tocopherols in the seed endosperm is modified. Preferably, a gene encoding an enzyme selected from VTE1, VTE3 and VTE4 is expressed operably under control of the promoter, active fragment or derivative, and a different gene of the tocopherol biosynthetic pathway is expressed operably under the control of another promoter in the endosperm e.g., by gene stacking.

In another example, a gene encoding a homogentisate geranylgeranyl transferase (HGGT) enzyme is expressed operably under control of the promoter, active fragment or derivative of the present invention to modulate the level of a tocotrienol in the endosperm. In another example, the expression of transgenes encoding HGGT and VTE3 and VTE4 polypeptides is regulated in the endosperm wherein at least one of said transgenes is operably under control of a promoter, active fragment or derivative of the present invention. Further examples of tocopherol biosynthetic enzymes, the expression of which is modulated using a promoter of the present invention, include, for example, tyrA, slr1736, ATPT2, dxs, dxr, GGPPS, HPPD, GMT, MT1, tMT2, AANT1, sir 1737 (Kridl et al., Seed Sci. Res. 1:209:219 (1991); Keegstra, Cell 56(2):247-53 (1989); Nawrath et al., Proc. Natl. Acad. Sci. U.S.A. 91:12760-12764 (1994); Xia et al., J. Gen. Microbiol. 138:1309-1316 (1992); Lois et al., Proc. Natl. Acad. Sci. U.S.A. 95 (5):2105-2110 (1998); Takahashi et al. Proc. Natl. Acad. Sci. U.S.A. 95 (17), 9879-9884 (1998); Norris et al., Plant Physiol. 117:1317-1323 (1998); Bartley and Scolnik, Plant Physiol. 104:1469-1470 (1994); Smith et al., Plant J. 11: 83-92 (1997); WO 00/32757; WO 00/10380; Saint Gully et al., Plant Physiol., 100(2):1069-1071 (1992); Sato et al., J. DNA Res. 7 (1):31-63 (2000)).

In yet another example, the level of plant proteins, particularly modified proteins that improve the nutrient value of the plant, is increased by expressing one or more proteins having enhanced nutritional value or content of specific amino acids in the endosperm operably under control of a promoter of the present invention or an active fragment or derivative thereof. For example, hordothionin protein modifications are described in WO 94/16078; WO 96/38562; WO 96/38563 and U.S. Pat. No. 5,703,409. U.S. Pat. No. 6,127,600 and U.S. Pat. No. 6,080,913 also describe transgenes for increasing accumulation of essential amino acids in seeds. Lysine-enriched and/or sulfur-enriched albumins are also described in WO 97/35023 and U.S. Pat. No. 5,990,389 and U.S. Pat. No. 5,885,802 (high methionine) and U.S. Pat. No. 5,939,599 (high sulfur) and US Pat. No. 5,912,414 (increased methionine). U.S. Pat. No. 6,459,019 describes transgenes for increasing lysine and threonine content, and WO96/01905 describes transgenes for increasing threonine content. Examples of amino acid biosynthetic enzymes include anthranilate synthase (e.g., as described in U.S. Pat. No. 5,965,727, PCT Publications WO 97/26366, WO 99/11800, and WO 99/49058), tryptophan decarboxylase (e.g., as described in PCT Publication WO 99/06581), threonine decarboxylase (e.g., as described in U.S. Pat. Nos. 5,534,421, and 5,942,660; PCT Publication WO 95/19442), threonine deaminase (PCT Publications WO 99/02656 and WO 98/55601), dihydrodipicolinic acid synthase (e.g., as described in U.S. Pat. No. 5,258,300), diacylglycerol acyltransferase (e.g., as described in U.S. Patent Publications 20030115632A1 and 20030028923A1), and aspartate kinase (e.g., as described in U.S. Pat. Nos. 5,367,110, 5,858,749, and 6,040,160).

In yet another example, altered carbohydrate metabolism is effected, for example, by altering expression of a gene for an enzyme that affects the branching pattern of starch or a gene altering thioredoxin such as NTR and/or TRX (e.g., U.S. Pat. No. 6,531,648) and/or Bacillus subtilis levansucrase gene (e.g., Steinmetz, et al., (1985) Mol. Gen. Genet. 200:220) and/or an alpha-amylase gene (e.g., Pen, et al., (1992) Bio/Technology 10:292; Sogaard, et al., (1993) J. Biol. Chem. 268:22480) and/or a tomato invertase gene (Elliot, et al., (1993) Plant Mol. Biol. 21:515) and/or starch branching enzymes (e.g., U.S. Pat. Nos. 6,232,122 and 6,147,279 and PCT Publication WO 97/22703) including a maize endosperm starch branching enzyme II (Fisher, et al., (1993) Plant Physiol. 102:1045 and/or UDP-D-xylose 4-epimerase or Fragile-1 or Fragile-2 or Ref1 or HCHL or C4H gene (e.g., WO 99/10498) and/or an ADP-glucose pyrophosphorylase (AGP; e.g., U.S. Pat. No. 6,232,529. It is also within the scope of the invention to achieve indirect modification of fatty acid levels or composition by directly modifying starch or other carbohydrate content in view of the interrelationship of the starch and oil pathways, and vice versa.

In yet another example, the promoter of the present invention or an active fragment or derivative thereof is employed to modulate ethylene production and/or perception and/or endosperm apoptosis associated with ethylene production and/or perception. For example, by down-regulating ethylene production and/or reception, apoptosis of cereal endosperm is delayed or repressed e.g., Campbell and Drew, Planta 157:350-357 (1983); Drew et al, Planta 147:83-88 (1979); He et al., Plant Physiol. 112:1679-1685 (1996); Young et al., Plant Physiol. 119:737-751 (1997); Young and Gallie, Plant Mol. Biol. 39:915-926 (1999); Young and Gallie, Plant Mol. Biol. 42:397-414 (2000)). Ethylene perception in cereals most likely involves homologs of the Arabidopsis membrane-localized receptors ETR1, ERS1, ETR2, ERS2 and EIN4 (Chang et al., Science 262:539-544 (1993); Hua et al., Science 269:1712-1714 (1995), Hua et al., Plant Cell 10:1321-1332 (1998), Sakai et al., Proc. Natl. Acad. Sci. USA 95:5812-5817 (1998)), or products of the maize ethylene receptor genes ZmETR2 and ZmERS1, ZmETR9 and ZmETR40. The endosperm of cereals serves as the major storage organ for grain but undergoes cell death during mid to late seed development, regulated by ethylene. By down-regulating expression of an ethylene receptor gene in the endosperm, apoptosis of the organ is delayed or reduced or suppressed, thereby extending the period of grain filling and storage protein deposition.

In another example, a promoter, active fragment or derivative as described herein according to any embodiment is used to express a therapeutic protein, such as, for example, a vaccine or an antibody fragment. Improved ‘plantibody’ vectors (e.g., as described in Hendy et al. J. Immunol. Methods 231:137-146, 1999) and purification strategies render such a method a practical and efficient means of producing recombinant immunoglobulins, not only for human and animal therapy, but for industrial applications as well (e.g., catalytic antibodies). Moreover, plant produced antibodies have been shown to be safe and effective and avoid the use of animal-derived materials and therefore the risk of contamination with a transmissible spongiform encephalopathy (TSE) agent. Furthermore, the differences in glycosylation patterns of plant and mammalian cell-produced antibodies have little or no effect on antigen binding or specificity. In addition, no evidence of toxicity or human anti-mouse antibody (HAMA) has been observed in patients receiving topical oral application of a plant-derived secretory dimeric IgA antibody (see Larrick et al. Res. Immunol. 149:603-608, 1998).

For example, a promoter of the present invention or an active fragment or derivative thereof is employed to express a recombinant antibody in the endosperm e.g., an anti-CD4 antibody capable of inhibiting HIV-1 virus-to-cell or infected cell-to-uninfected cell transmission or for suppressing or reducing an inflammatory response or for treatment of CD-4 autoimmune disorders such as rheumatoid arthritis or psoriasis.

Various methods may be used to express recombinant antibodies in transgenic plants. For example, antibody heavy and light chains can be independently cloned into a nucleic acid construct, followed by the transformation of plant cells in vitro using the method of the invention. Subsequently, whole plants expressing individual chains are regenerated followed by their sexual cross, ultimately resulting in the production of a fully assembled and functional antibody (see, for example, Hiatt et al. Nature 342:76-87, 1989). In various examples, signal sequences may be utilized to promote the expression, binding and folding of unassembled antibody chains by directing the chains to the appropriate plant environment.

In another example, a transgene encoding a peptide or polypeptide capable of eliciting an immune response in a host is linked to a promoter, active fragment or derivative as described herein according to any embodiment. For example, a transgene encoding Hepatitis B surface antigen is inserted into a nucleic acid construct described herein and used to produce a transgenic plant using a method described herein according to any embodiment. In accordance with this embodiment, a food product produced using the plant or a part thereof is then administered to humans (e.g., fed to a human) as a medicinal foodstuff or oral vaccine.

Without detracting from the general applicability of the promoter, active fragment or derivative of the invention, the present invention also encompasses linking said promoter, active fragment or derivative to a nucleic acid that encodes a protein that confers or enhances protection against a plant pathogen, such as, for example, a seed-borne fungus, seed-borne virus, seed-borne bacterium, or insect that feeds on the seed. Such proteins are known to those skilled in the art and include, for example, a range of structurally and functionally diverse plant defense proteins or pathogenesis-related proteins (e.g., chitinase, in particular acid chitinase or endochitinase; beta-glucanase in particular beta9-1,3-glucanase; ribosome-inactivating protein (RIP); a-kafirin polypeptide e.g., α-kafirin, β-kafirin, γ-kafirin; Hevea brasiliensis hevein; potato win1 or win2 proteins, or related protein from wheat such as, for example, wheatwin or WPR4 or, related protein from barley such as, for example, barwin); thionin, in particular K-thionin; thaumatin or thaumatin-like protein such as zeamatin; a proteinase inhibitor such as, for example, trypsin or chymotrypsin; or sormatin), virus coat proteins, and proteins that convert one or more pathogen toxins to non-toxic products. Nucleic acid encoding such proteins are publicly available and/or described in the scientific literature. The structures of such genes and their encoded proteins are fully described in the database of the National Center for Biotechnology Information of the US National Library of Medicine, 8600 Rockville Pike, Bethesda, Md. 20894, USA.

A promoter or active fragment or derivative as described herein according to any embodiment may also be placed in operable connection with a nucleic acid encoding a polypeptide for recombinant production of that polypeptide. As discussed supra, tissues of plant seeds, e.g., a dormant embryo, are useful for the production of recombinant polypeptides. Accordingly, the present invention provides a method for producing a recombinant polypeptide, e.g., for commercial purposes.

It is to be understood that the present invention also extends to the production of transgenic plants that express transgenes that do not encode a protein. For example, the transgene encodes an interfering RNA, an antisense RNA, a ribozyme, an abzyme, co-suppression molecule, gene-silencing molecule or gene-targeting molecule, which prevents or reduces the expression of a nucleic acid of interest.

Suitable methods for producing interfering RNA or a ribozyme, or an abzyme are known in the art.

For example, a number of classes of ribozymes have been identified. One class of ribozymes is derived from a number of small circular RNAs that are capable of self-cleavage and replication in plants. Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of transgenes encoding a ribozyme capable of selectively cleaving a target RNA is described, for example, in Haseloff et al. Nature, 334:585-591 (1988).

Alternatively, a transgene expresses a nucleic acid capable of inducing sense suppression of a target nucleic acid. For example, a transgene is produced comprising nucleic acid configured in the sense orientation as a promoter of a target nucleic acid. Such a method is described, for example, in Napoli et al., The Plant Cell 2:279-289 1990; or U.S. Pat. No. 5,034,323.

To reduce or prevent expression of a nucleic acid by sense suppression, the transgene need not be absolutely identical to the nucleic acid. Furthermore, the transgene need not comprise the complete sequence of the nucleic acid to reduce or prevent expression of said nucleic acid by sense-suppression.

RNA interference is also useful for reducing or preventing expression of a nucleic acid. Suitable methods of RNAi are described in Marx, Science, 288:1370-1372, 2000. Exemplary methods for reducing or preventing expression of a nucleic acid are described in WO 99/49029, WO 99/53050 and WO0/75164. Briefly a transgene is produced that expresses a nucleic acid that is complementary to a sequence of nucleotides in the target nucleic acid. The transgene additionally expresses nucleic acid substantially identical to said sequence of nucleotides in the target nucleic acid. The two nucleic acids expressed by the transgene are capable of hybridizing and reducing or preventing expression of the target nucleic acid, presumably at the post-transcriptional level.

MicroRNA or miRNA is a small double stranded RNA that regulates or modulates the expression of target messenger RNAs either by mRNA cleavage, translational repression/inhibition or heterochromatic silencing (see for example Ambros, 2004, Nature, 431, 350-355; Bartel, 2004, Cell, 116, 281-297; Cullen, 2004, Virus Research., 102, 3-9; He et al., 2004, Nat. Rev. Genet., 5, 522-531; and Ying et al., 2004, Gene, 342, 25-28). Such microRNA can be expressed using a promoter, active fragment or derivative as described herein according to any embodiment. Alternatively, a nucleic acid is capable of conferring expression or a pattern of expression on a miRNA using a promoter, active fragment or derivative as described herein according to any embodiment.

### Plant Transformation or Transfection

Following production of a suitable expression construct or expression vector the construct or vector is introduced into a plant cell or tissue. Means for introducing recombinant DNA into plant tissue or cells include, but are not limited to, transformation using CaCl₂ and variations thereof, e.g., as described by Hanahan (1983), direct DNA uptake into protoplasts (Krens et al, Nature 296, 72-74, 1982; Paszkowski et al., EMBO J. 3, 2717-2722, 1984), PEG-mediated uptake to protoplasts (Armstrong et al., Plant Cell Rep. 9, 335-339, 1990) microparticle bombardment, electroporation (Fromm et al., Proc. Natl. Acad. Sci. (USA), 82, 5824-5828, 1985), microinjection of DNA (Crossway et al., Mol. Gen. Genet. 202, 179-185, 1986), microparticle bombardment of tissue explants or cells (Christou et al, Plant Physiol. 87, 671-674, 1988; Sanford, Part. Sci. Technol. 5, 27-37, 1988), vacuum-infiltration of tissue with nucleic acid, or in the case of plants, T-DNA-mediated transfer from 30 Agrobacterium to the plant tissue as described essentially by An et al., EMBO J. 4, 277-284, 1985; Herrera-Estrella et al., Herrera-Estella et al., Nature 303, 209-213, 1983; Herrera-Estella et al., EMBO J. 2, 987-995, 1983; or Herrera-Estella et al., In: Plant Genetic Engineering, Cambridge University Press, N.Y., pp 63-93, 1985.

Particle bombardment-mediated transformation also delivers naked nucleic acid into plant cells (Sanford et al., J. Part. Sci. Technol. 5: 27, 37, 1987). This technique involves the acceleration of dense nucleic acid-coated microparticles, e.g., gold or tungsten particles, to a sufficient velocity to penetrate the plant cell wall and nucleus. The introduced nucleic acid is then incorporated into the plant genome, thereby producing a transgenic plant cell. This cell is then used to regenerate a transgenic plant. Exemplary apparatus and procedures are disclosed by Stomp et al. (U.S. Pat. No. 5,122,466) and Sanford and Wolf (U.S. Pat. No. 4,945,050). Suitable methods are also exemplified herein. Examples of microparticles suitable for use in such systems include 1 to 5 micron gold spheres. The DNA construct may be deposited on the microparticle by any suitable technique, such as by precipitation.

Alternatively, an expression construct or expression vector is introduced into a plant protoplast. To produce a protoplast, it is necessary to remove the cell wall from a plant cell. Methods for producing protoplasts are known in the art and described, for example, by Potrykus and Shillito, Methods in Enzymology 118, 449-578, 1986. Naked nucleic acid (i.e., nucleic acid that is not contained within a carrier, vector, cell, bacteriophage or virus) is introduced into a plant protoplast by physical or chemical permeabilization of the plasma membrane of the protoplast (Lörz et al., Mol. Gen. Genet. 199: 178-182, 1985 and Fromm et al., Nature, 319: 791-793, 1986).

The preferred physical means for introducing nucleic acid into protoplasts is electroporation, which comprises the application of brief, high-voltage electric pulses to the protoplast, thereby forming nanometer-sized pores in the plasma membrane. Nucleic acid is taken up through these pores and into the cytoplasm. Alternatively, the nucleic acid may be taken up through the plasma membrane as a consequence of the redistribution of membrane components that accompanies closure of the pores. From the cytoplasm, the nucleic acid is transported to the nucleus where it is incorporated into the genome.

The preferred chemical means for introducing nucleic acid into protoplasts utilizes polyethylene glycol (PEG). PEG-mediated transformation generally comprises treating a protoplast with nucleic acid of interest in the presence of a PEG solution for a time and under conditions sufficient to permeabilize the plasma membranes of the protoplast. The nucleic acid is then taken up through pores produced in the plasma membrane and either maintained as an episomal plasmid or incorporated into the genome of the protoplast.

In another example of this invention, the expression vector or construct is introduced into a plant cell by electroporation (Fromm et al., Proc. Natl. Acad. Sci. USA 82:5824, 1985). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells with the transformed gene can be accomplished using phenotypic markers.

Cauliflower mosaic virus (CaMV) is also useful as a vector for introducing an expression vector or construct into plant cells (Hohn et al., (1982) “Molecular Biology of Plant Tumors,” Academic Press, New York, pp. 549-560; Howell, U.S. Pat. No. 4,407,956). CaMV viral DNA genome is inserted into a parent bacterial plasmid creating a recombinant DNA molecule that can be propagated in bacteria. After cloning, the recombinant plasmid is again cloned and further modified by introduction of the desired nucleic acid. The modified viral portion of the recombinant plasmid is then excised from the parent bacterial plasmid, and used to inoculate the plant cells or plants.

A further method for introducing an expression construct into plant cells is to infect a plant cell, an explant, a meristem or a seed with Agrobacterium tumefaciens transformed with the expression construct. Under appropriate conditions known in the art, the transformed plant cells are grown to form shoots, roots, and develop further into plants. The expression construct is introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., Proc. Natl. Acad. Sci. USA 80:4803, 1984).

There are presently at least three different ways to transform plant cells with Agrobacterium: (1) co-cultivation of Agrobacterium with cultured isolated protoplasts; (2) transformation of cells or tissues with Agrobacterium, or (3) transformation of seeds, apices or meristems with Agrobacterium.

Method (1) uses an established culture system that allows culturing protoplasts and plant regeneration from cultured protoplasts.

Method (2) implies (a) that the plant cells or tissues can be transformed by Agrobacterium and (b) that the transformed cells or tissues can be induced to regenerate into whole plants.

Method (3) uses micropropagation. In the binary system, to have infection, two plasmids are needed: a T-DNA containing plasmid and a vir plasmid. Any one of a number of T-DNA containing plasmids can be used, the main issue being that one be able to select independently for each of the two plasmids.

After transformation of the plant cell or plant, those plant cells or plants transformed by the Ti plasmid so that the desired DNA segment is integrated can be selected by an appropriate phenotypic marker expressed by the transformation vector. These phenotypic markers include, but are not limited to, antibiotic resistance, herbicide resistance or a trait detectable by visual observation. Other phenotypic markers are known in the art and may be used in this invention.

Alternatively, the transformed plants are produced by an in planta transformation method using Agrobacterium tumefaciens, such as, for example, the method described by Bechtold et al., CR Acad. Sci. (Paris, Sciences de la vie/Life Sciences) 316, 1194-1199, 1993 or Clough et al., Plant J 16: 735-74, 1998, wherein A. tumefaciens is applied to the outside of the developing flower bud and the binary vector DNA is then introduced to the developing microspore and/or macrospore and/or the developing seed, so as to produce a transformed seed. Those skilled in the art will be aware that the selection of tissue for use in such a procedure may vary, however it is preferable generally to use plant material at the zygote formation stage for in planta transformation procedures.

In a further example, a graminaceous plant is transformed using a method comprising contacting a mature embryo, e.g., a wheat embryo from a seed that has completed grain filling, with an Agrobacterium comprising an expression vector for a time and under conditions sufficient for the expression vector to be delivered to one or more cells of the mature embryo. Such transformation may additionally comprise removing the seed coat and or performing the transformation in the presence of Soytone™, both of which improve transformation efficiency. The transformed cells may be used to regenerate a plant or plant part.

The present invention also encompasses products of repeated cycles of transformation employing transformed plant cells or plant parts comprising a promoter, active fragment or derivative of the present invention or a transgene placed operably under the control of said promoter, active fragment or derivative or a gene construct comprising said transgene operably under the control of said promoter, active fragment or derivative.

In one example, gene stacking is performed sequentially or simultaneously. In one example of simultaneous gene stacking, a plant cell, plant tissue, plant organ or whole plant is transformed with two gene constructs wherein at least one of said gene constructs comprises a promoter, active fragment or derivative or transgene or gene construct of the present invention. In an example of sequential gene stacking, a transformed first plant cell comprising a first promoter, active fragment or derivative or transgene or gene construct is transformed with a second gene construct different to that used to produce the first plant cell, tissue, organ or whole plant e.g., wherein the second gene construct comprises a second transgene placed operably under the control of a second promoter that is different to the first promoter of the first plant cell, tissue, organ or whole plant. For example, the second gene construct or second transgene may comprise a second promoter, active fragment or derivative of the present invention different to a first promoter, active fragment or derivative of the invention present in the first plant cell, tissue, organ or plant. In another example, the second promoter is operable in the seed, preferably in the endosperm of a plant e.g., a promoter that confers or regulates expression in a number of different plant organs, tissues or cells, e.g., including the endosperm, or regulates such expression predominantly or exclusively in the endosperm, including early endosperm and/or maturing endosperm. In another example, the second promoter is operable in the embryo of plant seed. In another example, the second gene construct may further comprise a second transgene different to the first transgene i.e., wherein the promoters regulating each transgene are different. For example, the first and second transgenes are utilized to express functionally distinct or structurally distinct or unrelated first and second structural genes or transgenes. Such different transgenes may catalyse or regulate different steps in the same biochemical pathway, or entirely different biochemical pathways, and/or they may act in concert i.e., cooperatively to produce one or more desired traits. Preferably, different selectable markers are used to monitor the first and second and subsequent transformations.

Specific examples of first and second transgenes for such gene stacking approaches will be apparent from the disclosure herein of exemplary promoters that may be used in combination with a promoter, active fragment or derivative of the present invention, and the disclosure herein of exemplary transgenes that may be expressed in plants e.g., operably under the control of a promoter, active fragment or derivative of the present invention. It is to be understood that, in gene stacking approaches, the description of transgenes that may be expressed in plants e.g., operably under the control of a promoter, active fragment or derivative of the present invention apply mutatis mutandis to second gene constructs and second transgenes of this example.

Regeneration and Propagation of a Plant from a Transformed Cell/Plastid

A whole plant may be regenerated from the transformed or transfected cell, in accordance with procedures known in the art. Plant tissue capable of subsequent clonally propagation, whether by organogenesis or embryogenesis, may be transformed with a vector or construct as described herein according to any embodiment.

The term “organogenesis”, as used herein, means a process by which shoots and roots are developed sequentially from meristematic centres.

The term “embryogenesis”, as used herein, means a process by which shoots and roots develop together in a concerted fashion (not sequentially), whether from somatic cells or gametes.

Plant regeneration from cultural protoplasts is described, for example, in Evans et al., “Protoplast Isolation and Culture—Handbook of Plant Cell Cultures 1” (MacMillan Publishing Co., 1983) and Binding “Regeneration of Plants”—Plant Protoplasts, pp 21-73 (CRC Press, Boca Raton, 1985). Regeneration varies from species to species. Generally, a suspension of transformed protoplasts is produced (e.g., using a method described herein). In some species the transformed protoplast is then induced to form an embryo and then to the stage of ripening and germination. Such induction involves, for example, the addition of compounds to the culture media of the protoplast, for example, glutamic acid and/or proline in the case of corn or alfalfa.

In an example, a plant or a plant part or a plantlet is regenerated using a transformed graminaceous plant cell produced using a method described herein. Preferably, a transformed cell is contacted with a compound that induces callus formation for a time and under conditions sufficient for callus formation. Alternatively, or in addition, a transgenic plant cell is contacted with a compound that induces cell de-differentiation for a time and under conditions sufficient for a cell to de-differentiate. Alternatively, or in addition, a transgenic plant cell is contacted with a compound that induces growth of an undifferentiated cell for a time and under conditions sufficient for an undifferentiated cell to grow. Compounds that induce callus formation and/or induce production of undifferentiated and/or de-differentiated cells will be apparent to the skilled artisan and include, for example, an auxin, e.g., 2,4-D, 3,6-dichloro-o-anisic acid (dicambia), 4-amino-3,5,6-thrichloropicolinic acid (picloram) or thidiazuron (TDZ).

Such a medium may additionally comprise one or more compounds that facilitate callus formation/de-differentiation or growth of undifferentiated cells. For example, Mendoza and Kaeppler (In vitro Cell Dev. Biol., 38: 39-45, 2002) found that media comprising maltose rather than sucrose enhanced the formation of calli in the presence of 2,4-D.

Alternatively, or in addition, the embryonic cell is additionally contacted with myo-inositol. Studies have indicated that myo-inositol is useful for maintaining cell division in a callus (Biffen and Hanke, Biochem. J. 265: 809-814, 1990).

Similarly, casein hydrolysate appears to induce cell division in a callus and maintain callus morphogenetic responses. Accordingly, in another example, the embryonic graminaceous plant cell is additionally contacted with casein hydrolysate.

Suitable culture medium and methods for inducing callus formation and/or cell de-differentiation and/or the growth of undifferentiated cells from mature embryonic graminaceous plant cells are known in the art and/or described in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004 and Delporte et al., Plant Cell, Tissue and Organ Culture, 80: 139-149, 2005.

Following callus induction, cell de-differentiation and/or growth of undifferentiated cells, the plant cells and/or a cell derived therefrom (e.g., a callus derived therefrom or a de-differentiated or undifferentiated cell thereof) is contacted with a compound that induces shoot formation for a time and under conditions sufficient for a shoot to develop. Suitable compounds and methods for inducing shoot formation are known in the art and/or described, for example, in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004, Murashige and Skoog, Plant Physiol., 15: 473-479, 1962 or Kasha et al., (In: Gene manipulation in plant improvement II, Gustafson ed., Plenum Press, 1990). For example, a callus or an undifferentiated or de-differentiated cell is contacted with one or more plant growth regulator(s) that induces shoot formation. Examples of suitable compounds (i.e., plant growth regulators) include indole-3-acetic acid (IAA), benzyladenine (BA), indole-butyric acid (IBA), zeatin, a-naphthaleneacetic acid (NAA), 6-benzyl aminopurine (BAP), thidiazuron, kinetin, 21P or combinations thereof.

Suitable sources of media comprising compounds for inducing shoot formation are known in the art and include, for example, Sigma-Aldrich Pty Ltd (Sydney, Australia).

Alternatively, or in addition, the callus or an undifferentiated or de-differentiated cell is maintained in or on a medium that does not comprise a plant growth modulator for a time and under conditions sufficient to induce shoot formation and produce a plantlet.

At the time of shoot formation or following shoot formation the callus or an undifferentiated or de-differentiated cell is preferably contacted with a compound that induces root formation for a time and under conditions sufficient to initiate root growth and produce a plantlet.

Suitable compounds that induce root formation are known to the skilled artisan and include a plant growth regulator, e.g., as described supra.

Suitable methods for inducing root induction are known in the art and/or described in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004, Murashige and Skoog, Plant Physiol., 15: 473-479, 1962 or Kasha et al., (In: Gene manipulation in plant improvement II, Gustafson ed., Plenum Press, 1990).

In an example of the invention, a callus and/or de-differentiated cell and/or undifferentiated cell is contacted with media comprising zeatin for a time and under conditions sufficient to induce shoot formation and contacted with medium comprising NAA for a time and under conditions sufficient to induce root formation.

Plantlets are then grown for a period of time sufficient for root growth before being potted (e.g., in potting mix and/or sand) and being grown.

The generated transformed plants may be propagated by a variety of means, such as by clonal propagation or classical breeding techniques. For example, a first generation (or T1) transformed plant may be selfed to give homozygous second generation (or T2) transformant, and the T2 plants further propagated through classical breeding techniques. In this respect, the skilled artisan will be aware that the term “selfed” refers to the process of selfing, which is discussed supra.

The present invention also encompasses products of repeated cycles of transformation employing plant material transformed with a promoter, active fragment or derivative of the present invention or a transgene placed operably under the control of said promoter, active fragment or derivative or a gene construct comprising said transgene operably under the control of said promoter, active fragment or derivative.

In one example, gene stacking is performed. In one example of gene stacking, a first plant cell, first plant tissue or first plant organ or first whole plant comprising a first promoter, active fragment or derivative or transgene or gene construct is transformed with a second gene construct different to that used to produce the first plant cell, tissue, organ or whole plant e.g., wherein the second gene construct comprises a second transgene placed operably under the control of a second promoter that is different to the first promoter of the first plant cell, tissue, organ or whole plant. For example, the second gene construct or second transgene may comprise a second promoter, active fragment or derivative of the present invention different to a first promoter, active fragment or derivative of the invention present in the first plant cell, tissue, organ or plant. In another example, the second promoter is operable in the seed, preferably in the endosperm of a plant e.g., a promoter that confers or regulates expression in a number of different plant organs, tissues or cells, e.g., including the endosperm, or regulates such expression predominantly or exclusively in the endosperm, including early endosperm and/or maturing endosperm. In another example, the second promoter is operable in the embryo of plant seed. In another example, the second gene construct may further comprise a second transgene different to the first transgene i.e., wherein the promoters regulating each transgene are different. For example, the first and second transgenes are utilized to express functionally distinct or structurally distinct or unrelated first and second structural genes or transgenes. Such different transgenes may catalyse or regulate different steps in the same biochemical pathway, or entirely different biochemical pathways, and/or they may act in concert i.e., cooperatively to produce one or more desired traits.

Specific examples of first and second transgenes for such gene stacking approaches will be apparent from the disclosure herein of exemplary promoters that may be used in combination with a promoter, active fragment or derivative of the present invention, and the disclosure herein of exemplary transgenes that may be expressed in plants e.g., operably under the control of a promoter, active fragment or derivative of the present invention. It is to be understood that, in gene stacking approaches, the description of transgenes that may be expressed in plants e.g., operably under the control of a promoter, active fragment or derivative of the present invention apply mutatis mutandis to second gene constructs and second transgenes of this example.

The present invention also encompasses products of traditional breeding or asexual or clonal propagation employing plant material transformed with a promoter, active fragment or derivative of the present invention or a transgene placed operably under the control of said promoter, active fragment or derivative or a gene construct comprising said transgene operably under the control of said promoter, active fragment or derivative.

In one example, gene stacking is performed. In one example of gene stacking, a first plant comprising a first promoter, active fragment or derivative or transgene or gene construct is cross sexually with a second plant expressing one or more desired traits or having a desired genetic background, and progeny carrying the first promoter, active fragment or derivative or transgene or gene construct and expressing the desired trait(s) are identified and optionally, isolated. As will be known to those skilled in the art, if the parents of such a cross do not each contribute the same genetic material to their progeny, then such progeny plants are heterozygous for the parentally-derived first promoter, active fragment or derivative or transgene or gene construct and the desired trait(s). In another example, the heterozygous progeny are then selfed and the homozygous progeny identified and optionally, isolated. Where such crosses are intended to introgress a promoter, active fragment or derivative or transgene or gene construct of the invention into a desired genetic background, repeated backcrossing is performed between the progeny of each cross and a plant comprising the desired genetic background. Generally, sufficient backcrosses are performed to ensure that the introduced promoter, active fragment or derivative or transgene or gene construct of the primary transformant is present in a genetic background that is substantially or significantly the same as the desired genetic background.

In another example, the one or more desired traits present in a parent of such a breeding or crossing program is/are conferred by a second gene construct different to the first gene construct of the other parent or is conferred by a second transgene placed operably under the control of a second promoter that is different to the first promoter of the other parent. For example, the second gene construct or second transgene may comprise a second promoter, active fragment or derivative of the present invention different to the first promoter, active fragment or derivative. In another example, the second promoter is operable in the seed, preferably in the endosperm of a plant e.g., a promoter that confers or regulates expression in a number of different plant organs, tissues or cells, e.g., including the endosperm, or regulates such expression predominantly or exclusively in the endosperm, including early endosperm and/or maturing endosperm.

In another example, the second promoter is operable in the embryo of plant seed. In another example, the second gene construct may further comprise a second transgene different to the first transgene i.e., wherein the promoters regulating each transgene are different. For example, the first and second transgenes are utilized to express functionally distinct or structurally distinct or unrelated first and second structural genes or transgenes. Such different transgenes may catalyse or regulate different steps in the same biochemical pathway, or entirely different biochemical pathways, and/or they may act in concert i.e., cooperatively to produce one or more desired traits.

Specific examples of first and second transgenes for such gene stacking approaches will be apparent from the disclosure herein of exemplary promoters that may be used in combination with a promoter, active fragment or derivative of the present invention, and the disclosure herein of exemplary transgenes that may be expressed in plants e.g., operably under the control of a promoter, active fragment or derivative of the present invention. It is to be understood that, in gene stacking approaches, the description of transgenes that may be expressed in plants e.g., operably under the control of a promoter, active fragment or derivative of the present invention apply mutatis mutandis to second transgenes of this example.

As will be apparent from the foregoing, the present invention additionally provides progeny or reproductive tissue of a genetically modified cell or organism of the invention, subject to the proviso that the progeny or reproductive tissue comprises nucleic acid encoding the fusion protein of the invention.

The generated transformed organisms contemplated herein may take a variety of forms. For example, they may be chimeras of transformed cells and non-transformed cells; clonal transformants (e.g., all cells transformed to contain the expression construct or vector); grafts of transformed and untransformed tissues (e.g., in plants, a transformed root stock grafted to an untransformed scion).

### Identification of Additional Promoters

As discussed herein-above, the inventors have also provided a method for identifying or isolating a promoter capable of conferring expression or a pattern of expression on a nucleic acid, e.g., in developing endosperm of a plant or a cell or tissue thereof. In a preferred example, the method comprises:

(i) determining the level of expression of a plurality of expression products in a dormant embryo;
(ii) determining the level of expression of a plurality of expression products in control tissue or cell or plant part;
(iii) identifying one or more expression products expressed at an increased level at (i) compared to (ii); and
(iv) isolating a promoter that confers expression on the one or more expression products at (iii) in developing endosperm.

A suitable control plant part, tissue or cell will be apparent to the skilled artisan and include any plant part, tissue or cell that is not from a dormant embryo. Preferably, the control plant part, tissue or cell is from a non-dormant seed or embryo, e.g., from an imbibed embryo or seed or from a germinating embryo or seed.

Preferably, the expression products detected are transcripts or mRNA encoded by a gene. For example, the transcripts or mRNA are detected using a microarray.

In one example, the level of expression in a dormant embryo is compared to the level of expression in a plurality of control tissues, cells or plant parts. For example, the plurality of control tissues, cells or plant parts includes a plant part, tissue or cell is from a non-dormant seed or embryo and a non-embryonic plant part, non-embryonic tissue or non-embryonic cell. In this manner, a promoter that confers expression on a nucleic acid preferentially or selectively in developing endosperm or a cell or tissue thereof is identified.

In one example, the method as described herein according to any embodiment additionally comprises:

(v) optionally, determining the structure of the promoter, e.g., the sequence of the promoter;
(vi) optionally, providing the structure of the promoter; and
(vii) providing the promoter.

In one example, the promoter is provided in an expression vector. The present invention clearly extends to the direct product of any method of identification or isolation of a promoter described herein.

The present invention is further described with reference to the following non-limiting examples.

### Example 1

### Identification of Wheat Genes Expressed Selectively in Developing Wheat Seeds

This example provides support for the seed-selective expression of two wheat genes, which are regulated in their native context by the wheat promoters of the present invention designated WP05 and WP07.

Affymetrix GeneChip® Wheat Genome Arrays were interrogated with probes derived from different RNA samples (immature embryo, embryos from seeds imbibed for 24 hours or 48 hours) and candidate genes exhibiting a seed-specific expression profile were identified.

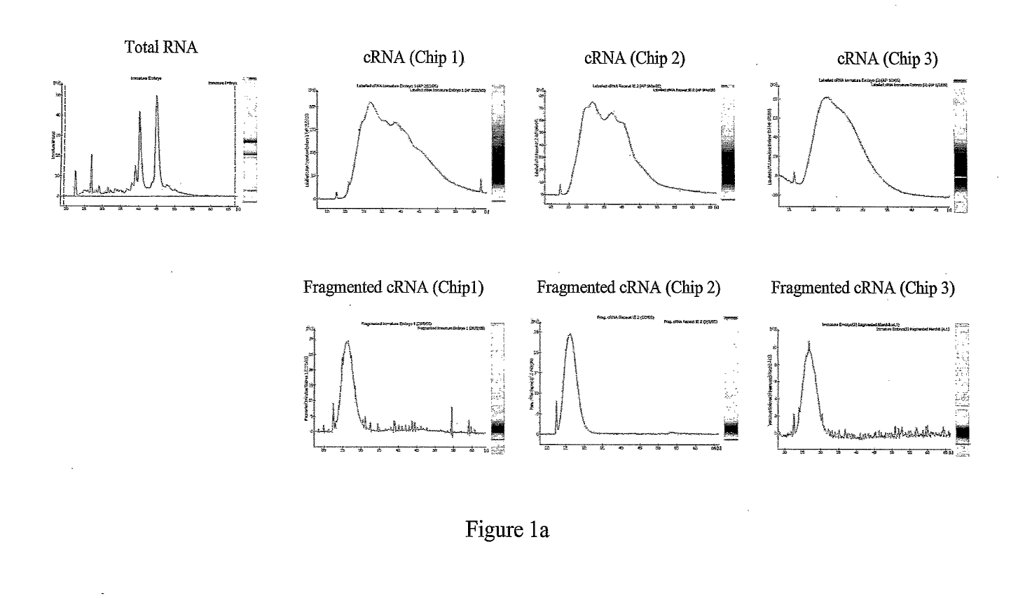

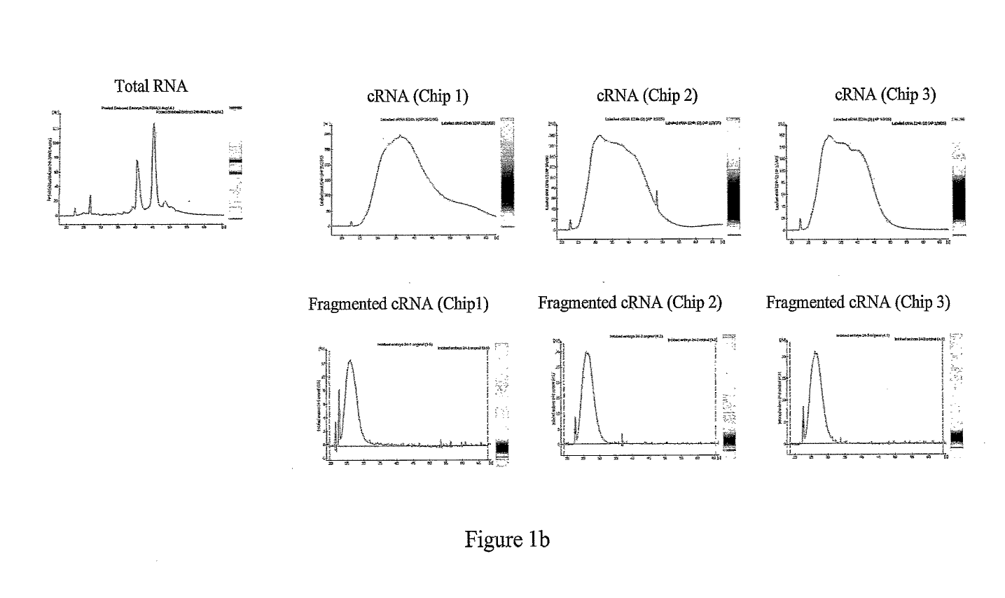

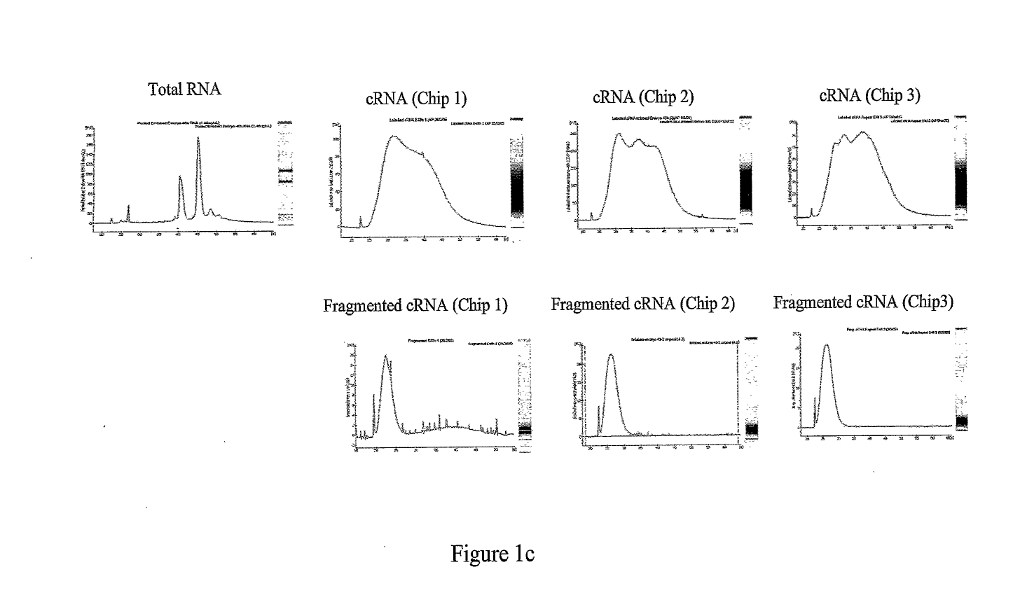

Immature wheat embryos (12-14 days post anthesis) and imbibed seed (24 hours or 48 hours) material were harvested, RNA extracted and further purified, and the quality and yield of RNA confirmed (FIGS. 1a, 1b, 1c). The RNA was labelled and hybridised to GeneChip® Wheat Genome Arrays and the data analysed to derive lists of genes in rank order.

Microarray expression was analysed using AVADIS™ software (Strand Genomics Pvt. Ltd. Bangalore). The raw data for all microarray analysis were imported into AVADIS and the RMA algorithm (Irazarry et al., Biostatistics 4(2): 249-264, 2003) was applied for background correction, normalisation and probe aggregation. Absolute calls and p-values were generated for each gene and all probe sets that did not hybridize to nucleic acid in a sample, i.e., were absent (absolute call), across all arrays were removed from the analysis.

For determination of transcripts preferentially or selectively expressed in seeds, two differential expression analyses were conducted where either immature embryo was compared to embryo that had been imbibed for 24 hours, or alternatively, immature embryo was compared to that had been imbibed for 48 hours. For the analysis of expression in immature embryo compared to 24 hr-imbibed embryo, only genes present (absolute call) in all immature embryo arrays and absent (absolute call) in the 24 hr-imbibed embryo were retained. For the analysis of expression in immature embryo compared to 48 hr-imbibed embryo, only genes present (absolute call) in all immature embryo arrays and absent (absolute call) in 48 hr-imbibed embryo were retained. The two datasets were exported to Excel and combined to create a list of genes expressed in immature embryo but not in either the 24 hr-imbibed or 48 hr-imbibed embryos. The mean, standard deviation and % CV of the fold change values were calculated. The gene list was ranked on the p-value of differential expression levels and filtered to retain only those genes expressed differentially by greater than 10-fold and more than 6000 the mean signal for expression in immature embryo.

Based on these criteria a list of candidate genes was prepared whose function was unknown, and for which no corresponding upstream genomic sequence was available in public domain databases.

Sequences for the candidate genes present on the Affymetrix GeneChip® Wheat Genome Arrays were obtained through the NetAffx web portal (http://wvvw.affymetrix.com/analysis/netaffx/index.affx).

The Affymetrix sequences and the corresponding public sequences from GenBank were downloaded and aligned using Sequencher™ software. In obvious cases, e.g. long stretches of poly-T at the start of the sequence, sequences were reverse-complemented to yield “sense” orientation, exported from Sequencher™ and consequently used for the primer design. In all other, non-obvious cases it was assumed that the sequences were in the “sense” orientation. The GenBank sequences were used as input files for primer design.

Primers for RT-QPCR validation were designed using the “TaqMan MGB probe and primer design” module of PrimerExpress™ version 1.5 used with the default settings. Two primer pairs were identified for each target candidate gene and internal standard.

RT-QPCR was performed using SYBR® Green fluorescence to detect amplification of candidate gene sequences from the cDNA samples used for the microarray experiments. A standard real-time PCR mixture for each candidate gene contained 1×SYBR® Green master mix, 200-300 nM of each primer, 2 μl of cDNA (about 20 ng) and water to a final volume of 25 μl. The thermo-cycling conditions for the PCR were: 1 cycle of 95° C. for 10 minutes followed by 40 cycles of 95° C. for 30 seconds, 60° C. for 1 minute. Real-time PCRs and data analysis was performed on a Stratagene MX3000p Real Time PCR machine. The dissociation protocol was used to demonstrate single amplicons with the correct Tm.

The sequence of one seed specific candidate gene from the Affymetrix clone Ta.10021.1_at, corresponding to clone wdk2c.pk009.e4:fis, a full insert mRNA sequence from Triticum aestivum (gb:BT008988.1/DB_XREF=gi:32128539/TID=Ta.10021.1/CNT=38/FEA=mRNA/TIER=ConsEnd/STK=1/UG=Ta.10021) is presented as SEQ ID NO: 1. The expression pattern of this gene was validated to be seed specific by RT-QPCR. For the purposes of nomenclature, the

The sequence of another seed specific candidate gene from the Affymetrix clone Ta.9233.2.S1 corresponding to the Tria27 mRNA for 27K protein (gb:CD906555/DB_XREF=gi:32680884/DB_XREF=G468.105B18R010929/CLONE=G468105B18/TID=Ta.9233.2/CNT=132/FEA=EST/TIER=Stack/STK=10/UG=Ta.9233) is presented as SEQ ID NO: 2. The expression pattern of this gene was also validated to be seed specific by RT-QPCR.

### Example 2

### Isolation of Endosperm-Selective Promoters from Wheat Genes Expressed Selectively in Developing Wheat Seeds

This example provides support for the isolation of the wheat-derived promoters of the present invention designated WP05 and WP07.

For the purposes of nomenclature, the promoter designated herein as “WP05” is operably linked in its native context to the Affymetrix clone Ta.10021.1, and the promoter designated herein as “WP07” is operably linked in its native context to the Affymetrix clone Ta.9233.2.S1.

To clone the promoter regions of the Affymetrix clones Ta.10021.1 and Ta.9233.2.S1, genome walking was performed using the Genome Walker™ kit available from Clontech Laboratories, Inc, (Mountain View, Calif., USA). Briefly, Genomic DNA was extracted from Triticum aestivum cultivar Bobwhite 26 and digested with the blunt end restriction enzymes SspI, ScaI, EcoRV, StuI, DraI. The resulting fragments were then used to create several Genome Walker™ libraries comprising wheat genomic DNA. Digested DNA was then purified with phenol chloroform and redissolved in TE buffer (10 mM Tris HCl, 0.1 mM EDTA, pH 7.5) and ligated to adaptors from the Genome Walker™ kit. The resulting libraries were designated:

### 1. DL 1—SspI

### 2. DL 2—DraI

### 3. DL 3—ScaI

### 4. DL 4—EcoRV

### 5. DL 5—StuI

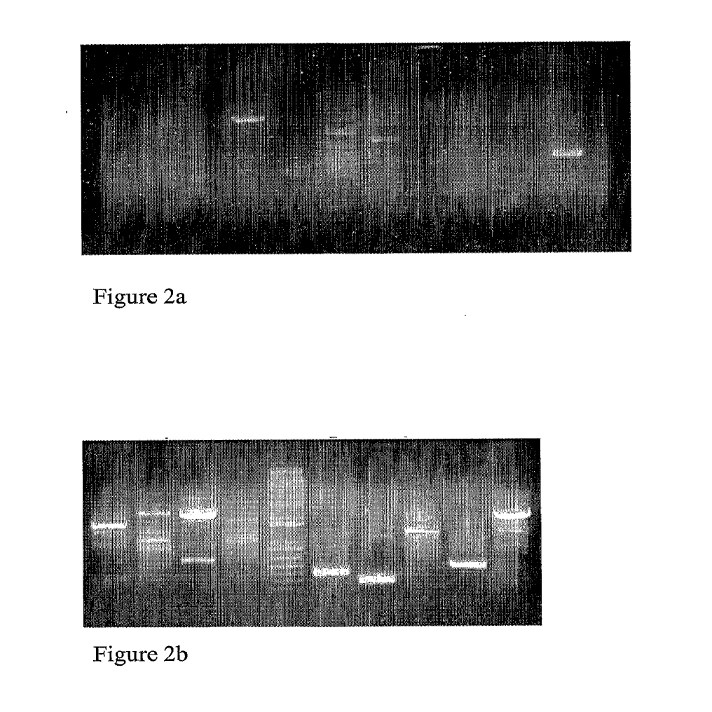

Nested PCR was performed on the wheat DNA library templates with adapter and sequence-specific primers. PCR products were resolved using electrophoresis using 0.7% (w/v) agarose gels (FIGS. 2a, 2b). Fragments with sizes around or greater than 1.0 kb in length were excised from the gels, purified and ligated into the vector pGEM-T Easy essentially according to manufacturer's instructions (Promega Corporation, Madison, Wis., USA). Fragments were sequenced and aligned with sequence data from Affymetrix and GenBank for each target candidate gene. Promoter sequences designated WP05 and WP07 were identified from alignments as those regions upstream of predicted open reading frames.

A total of 5 separate PCR amplification products were isolated for the Affymetrix clone Ta.10021.1.S1_at (Table 2), and the WP05 promoter fragment was determined to be localized in a 1.60 kb fragment (fragment WPRO5.2.1). A total of 6 separate PCR amplification products were isolated for the Affymetrix clone Ta.9233.2.S1_a_at (Table 2), and the WP07 promoter fragment was determined to be localized in a 2.70 kb fragment (fragment WPRO7.5.1).

The sequence of the WP05 promoter is set forth in SEQ ID NO: 3, and the sequences of two variants of the WP07 promoter are set forth in SEQ ID NOs: 4 and 5 (a 2400 bp variant and a 2066 bp variant, respectively).

TABLE 2

| | No Genome | | | |
|---|---|---|---|---|
| Affymetrix | Walker | Fragment | Fragment | Contig |
| Code | Bands | Codes | Size (kb) | result |
| Ta.10021.1.S1_at | 5 | WPR05.1.1 | 1.20 | |
| | | WPR05.2.1 | 1.60 | WP05 |
| | | | | promoter |
| | | WPR05.5.1 | 0.6 | |
| | | WPF05.1.1 | 1.60 | |
| | | WPF05.4.1 | 3.0 | |
| Ta.9233.2.S1_a_at | 5 | WPR07.1.1 | 1.0 | |
| | | WPR07.1.2 | 0.50 | |
| | | WPR07.2.1 | 2.50 | |
| | | WPR07.3.1 | 1.00 | |
| | | WPR07.4.1 | 2.10 | |
| | | WPR07.5.1 | 2.70 | WP07 |
| | | | | promoter |

### Example 3

### Validation of Functionality of Endosperm-Selective Promoters WP05 and WP07

This example provides support for the functionality of the isolated wheat-derived promoters of the present invention designated WP05 and WP07 in conferring expression selectively or specifically in endosperm of developing seeds, by virtue of the promoters regulating expression of a reporter gene selectively or specifically in developing endosperm of at least wheat and maize transformants.

### 1. Plant Transformation Methods

### a) Wheat Transformation Vectors

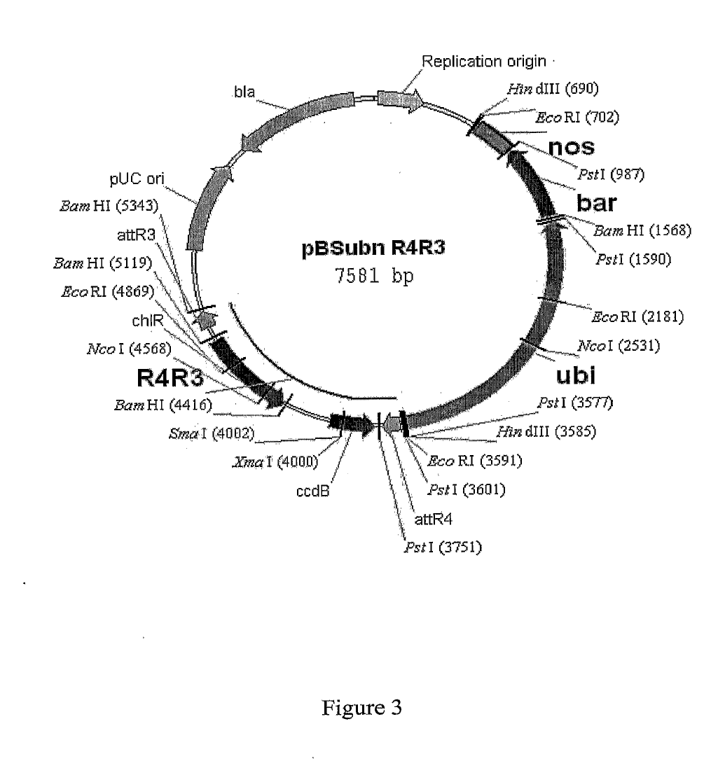

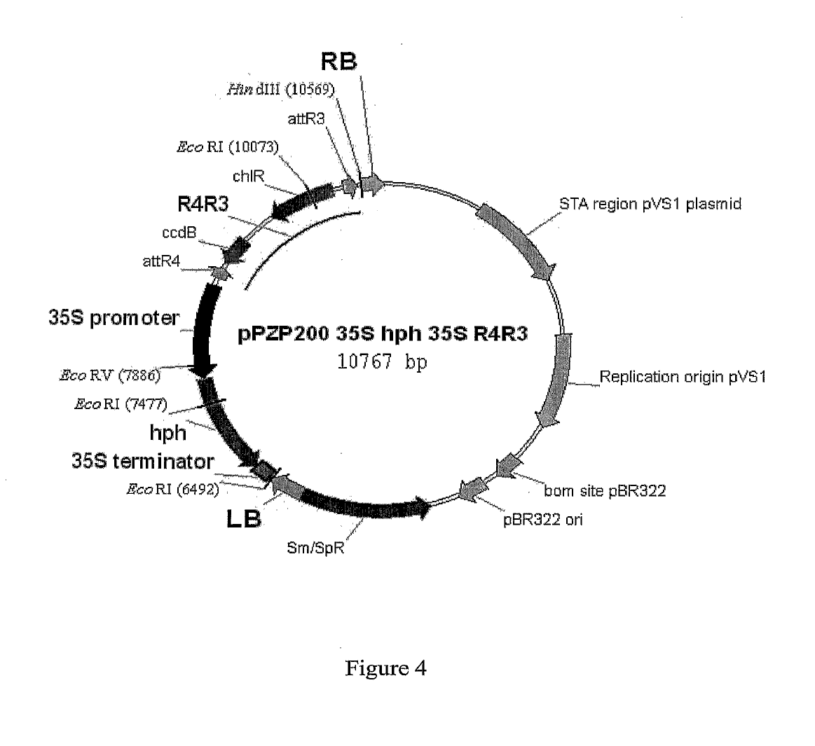

A base vector pBSubn R4R3 (FIG. 3; SEQ ID NO: 10) was used as a source of a selectable marker cassette wherein a ubiquitin promoter regulates expression of the bar selectable marker gene operably linked to the nopaline synthase (NOS) gene terminator i.e., Ubi::bar-nos. A base vector pPZP200 35D hph 35S R4R3 (FIG. 4; SEQ ID NO: 11) was used as a source of a selectable marker cassette wherein a CaMV 35S promoter regulates expression of the hygromycin phosphotransferase (hph) selectable marker gene operably linked to the CaMV 35S gene terminator i.e., 35S::hph-35S. Binary vectors were generated from the base vectors, for use in the transformation of plants. Briefly, reporter gene cassettes comprising each of the wheat promoters (SEQ ID NOs: 4-6) operably linked to the green fluorescent protein gene (gfp) and either CaMV 35S or NOS terminator were produced, amplified by PCR using Gateway™ (Invitrogen) adapted primers, and cloned into entry vectors. These were subsequently cloned using recombination into destination vectors containing the conventionally cloned selectable marker cassettes. All vectors were fully sequenced following strict quality assurance protocols.

Each binary vector produced has the pPZP200 vector backbone (Hajdukiewicz et al., Plant Mol. Biol. 25:989-94, 1994) and contains a chimeric reporter gene cassette and selectable marker cassette as follows:

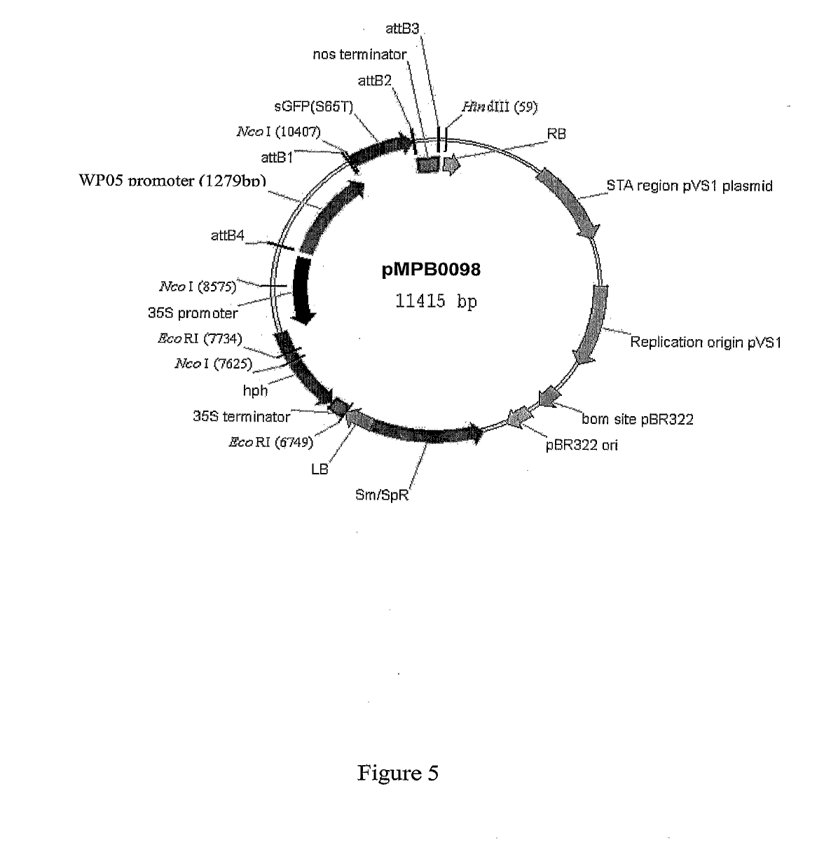

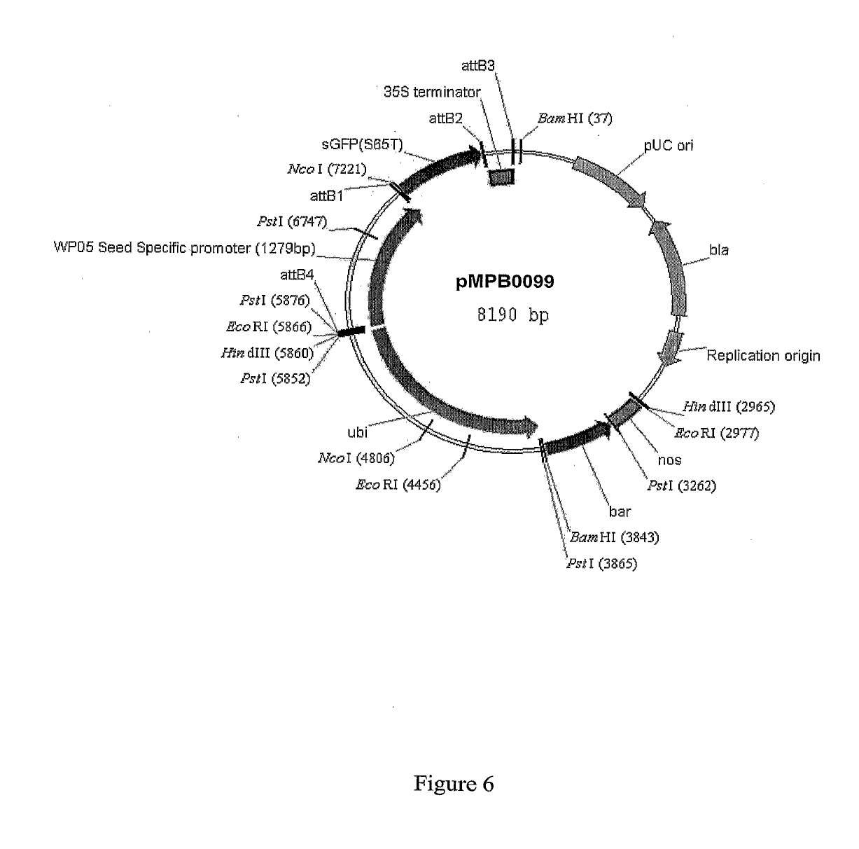

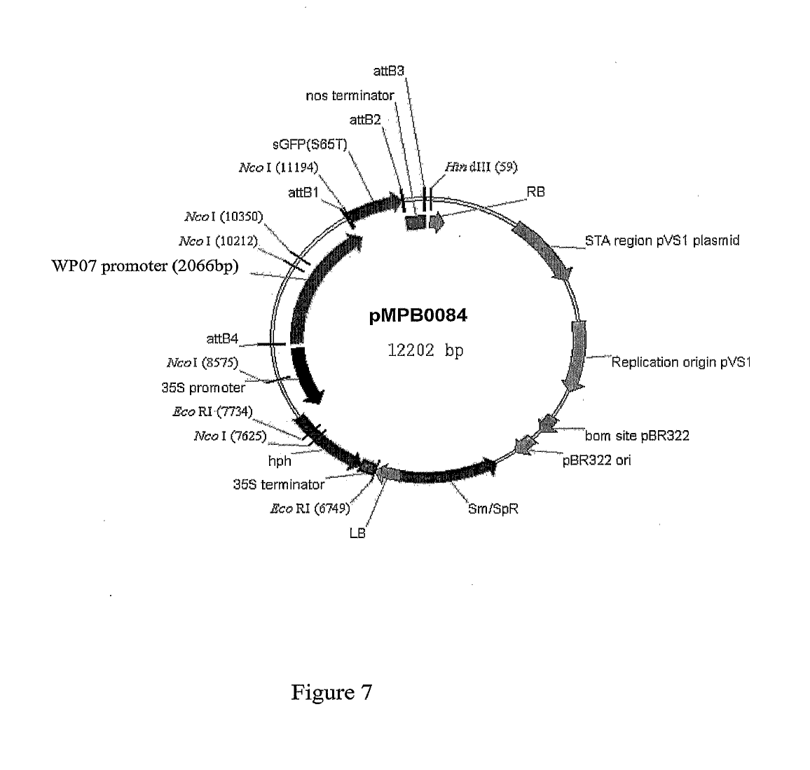

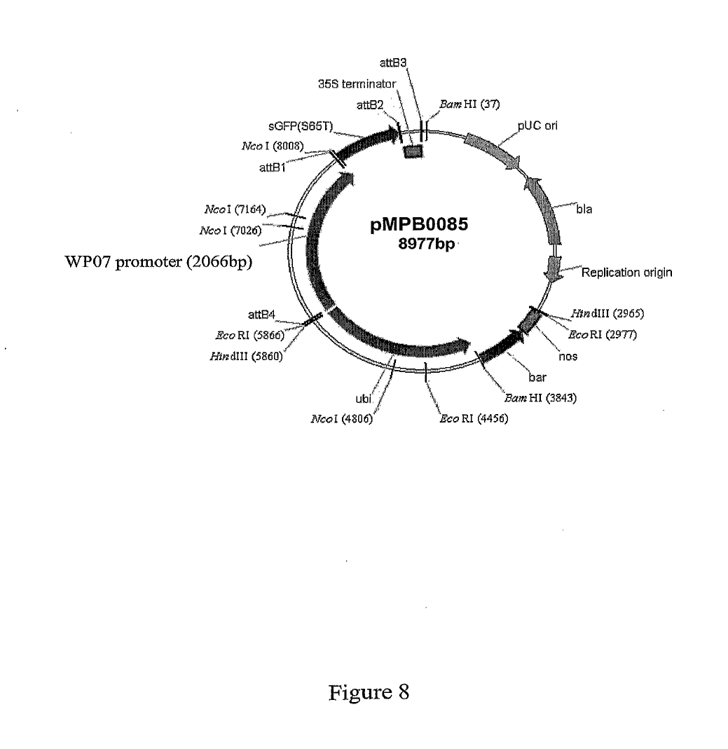

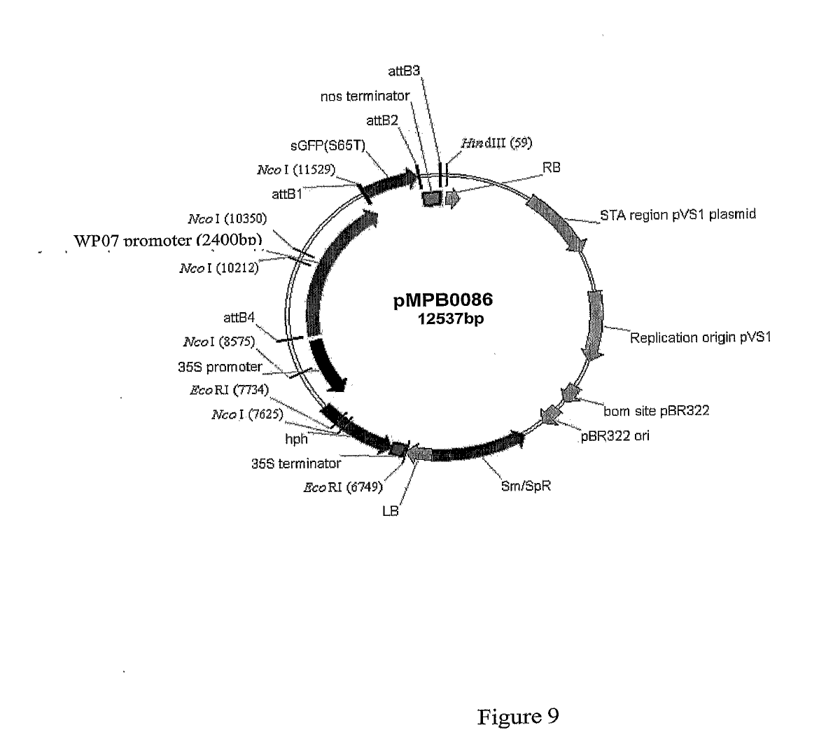

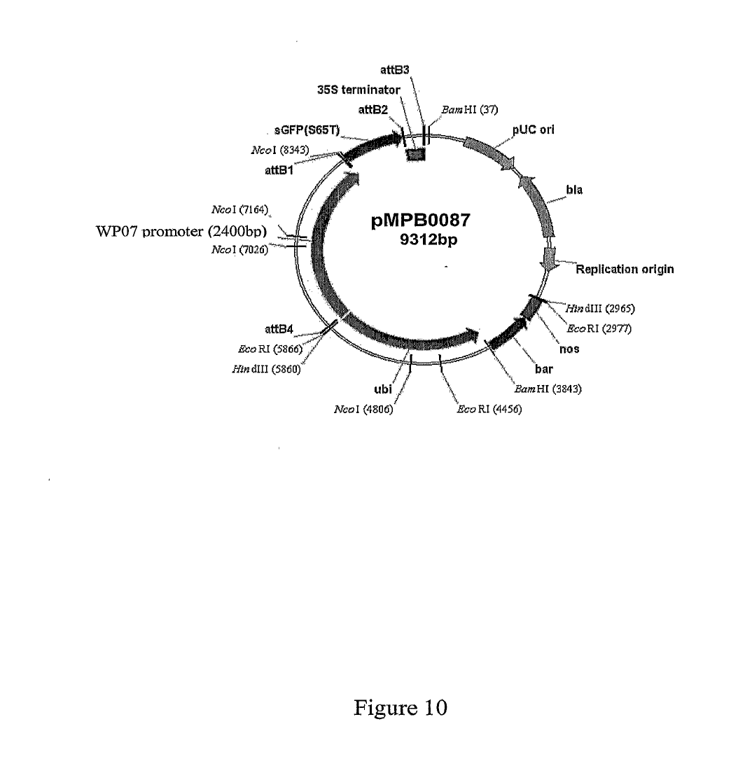

(i) WP05::sgfp-nos reporter gene cassette and 35S::hph-35S selectable marker cassette (pMPB0098; FIG. 5; SEQ ID NO: 12);
(ii) WP05::sgfp-nos reporter gene cassette and Ubi::bar-nos selectable marker cassette (pMPB0099; FIG. 6; SEQ ID NO: 13);
(iii) WP07::sgfp-nos reporter gene cassette wherein the WP07 promoter is the 2066 bp promoter fragment, and 35S::hph-35S selectable marker cassette (pMPB0084; FIG. 7; SEQ ID NO: 14);
(iv) WP07::sgfp-nos reporter gene cassette wherein the WP07 promoter is the 2066 bp promoter fragment, and Ubi::bar-nos selectable marker cassette (pMPB0085; FIG. 8; SEQ ID NO: 15);
(v) WP07::sgfp-nos reporter gene cassette wherein the WP07 promoter is the 2400 bp promoter fragment, and 35S::hph-35S selectable marker cassette (pMPB0086; FIG. 9; SEQ ID NO: 16); and
(vi) WP07::sgfp-nos reporter gene cassette wherein the WP07 promoter is the 2400 bp promoter fragment, and Ubi::bar-nos selectable marker cassette (pMPB0087; FIG. 10; SEQ ID NO: 17).

### b) Maize Transformation Vectors

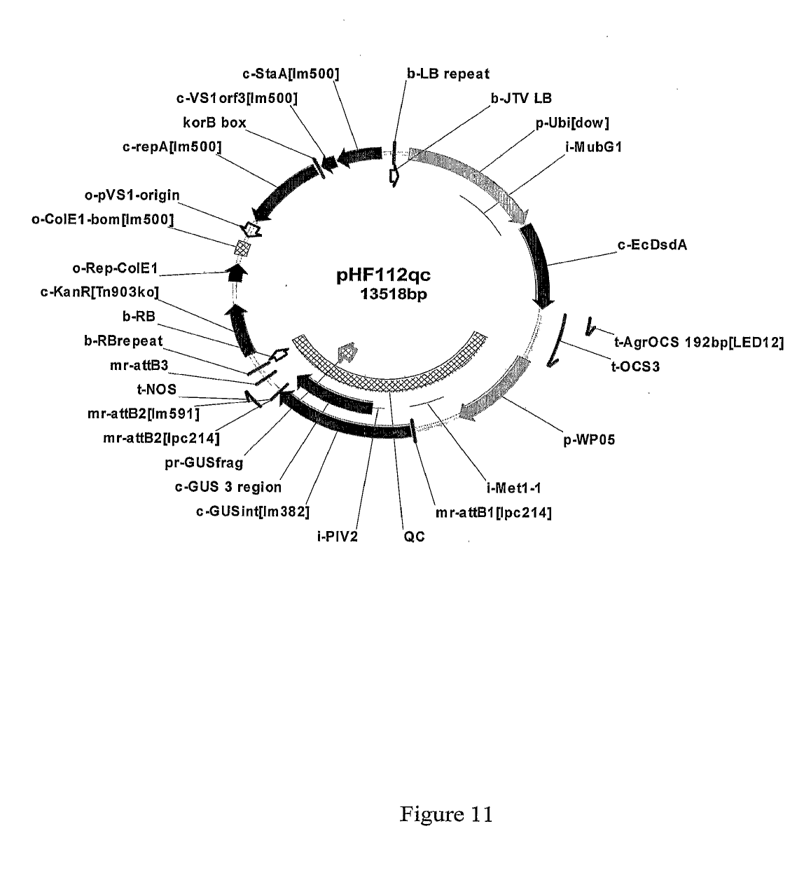

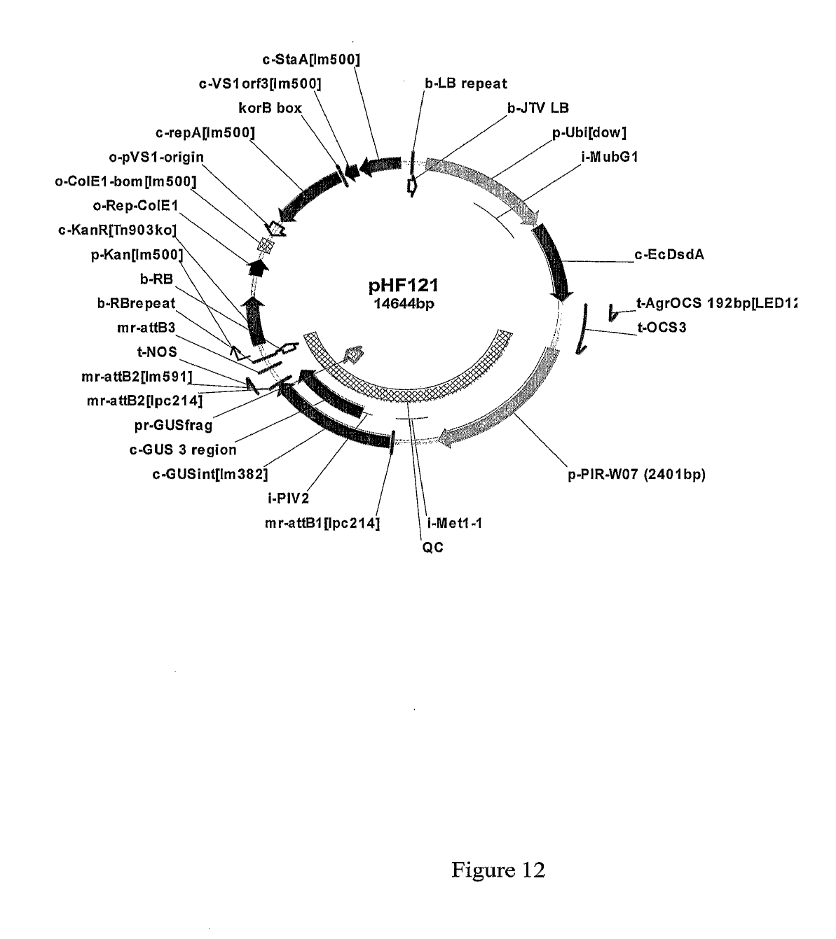

To generate expression vectors to validate functionality of the WP05 and WP07 promoters in maize, the promoters (SEQ ID Nos: 3 and 4) were amplified and cloned into pENTRTM 5′-TOPO TA Cloning vector (Invitrogen, Carlsbad, Calif., USA). The resulting vectors were used as Gateway entry vectors to generate the binary vectors RHF112 (FIG. 11; SEQ ID NO: 18) comprising the WP05 promoter regulating expression of the beta-glucuronidase (GUS) gene operably linked to a NOS gene terminator, and RHF121 (FIG. 12; SEQ ID NO: 19) comprising the 2400 bp WP07 promoter regulating expression of the beta-glucuronidase (GUS) gene operably linked to a NOS gene terminator.

c) Biolistic Transformation of Wheat (Triticum aestivum L)

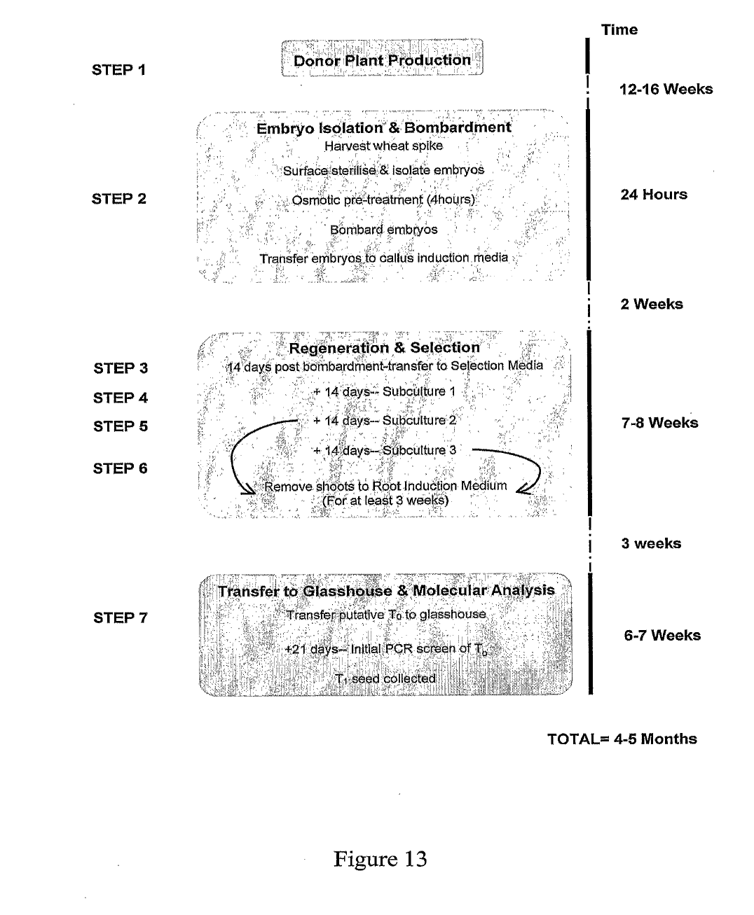

The wheat transformation vectors described herein above were used for biolistic transformation of wheat (Triticum aestivum L. MPB Bobwhite 26). A schematic of the transformation procedure is depicted in FIG. 13. The transformation procedure includes the following steps:

Step 1 (Donor Plant Production):

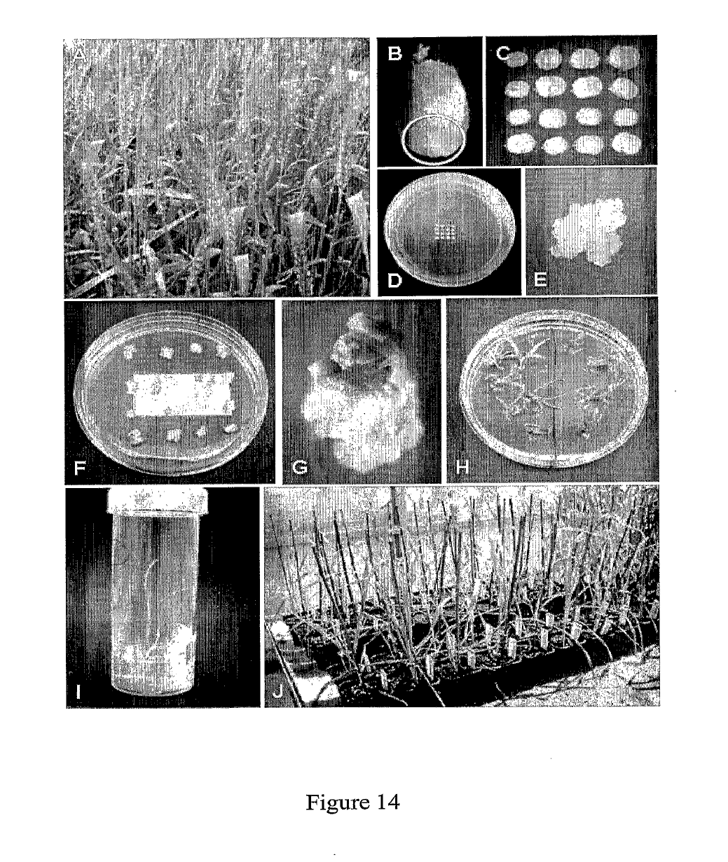

Triticum aestivum (Bobwhite 26) seed was used for the production of donor plant material. Wheat plants were grown in a nursery mix consisting of composted pine bark, perlite and vermiculite, with five plants per pot to a maximum pot size of 20 cm. Plants were kept under glasshouse conditions at approximately 22-24° C. for 12-16 weeks (FIG. 14a). Once the first spike emerged from the flag leaf, plants were tagged and embryos collected from the tallest heads 12-15 days post anthesis.

Step 2 (Day 1)

Spikes at the desired stage of development were harvested. Caryopses were removed from the spikes and surface sterilised for 20 minutes in a 0.8% (v/v) NaOCl solution and rinsed at least four times in sterile distilled water. Embryos up to 10 mm in length were aseptically excised from each caryopsis (removing the axis) using a dissecting microscope and cultured axial side down on an osmotic medium (E3maltose) consisting of 2× Murashige and Skoog (1962) macronutrients, 1× micronutrients and organic vitamins, 40 mg/L thiamine, 150 mg/L L-asparagine, supplemented with 15% (w/v) maltose, 0.8% (w/v) Sigma-agar and 2.5 mg/L 2,4-D. Embryos were cultured on 60 mm×15 mm clear polypropylene Petri dishes with 15 mL of media. Culture plates were incubated at 24° C. in the dark for 4 hours prior to bombardment. Embryos were bombarded using a BioRad PDS1000 gene gun at 900 psi and at 6 cm with 1 μg of vector plasmid DNA precipitated onto 0.6 μm gold particles. Following bombardment, embryos were incubated overnight in the dark on the osmotic media. This step is shown in FIGS. 14b, 14c and 14d.

Step 3 (Day 2):

Embryos were transferred to a callus induction medium (E3calli) consisting of 2× Murashige and Skoog (1962) macronutrients and 1× micronutrients and organic vitamins, 40 mg/L thiamine, 150 mg/L L-asparagine, supplemented with 6% (w/v) sucrose, 0.8% (w/v) Sigma-agar and 2.5 mg/L 2,4-D. Embryos were cultured for two weeks at 24° C. in the dark.

Step 4 (Day16):

After 2 weeks of culture on E3 calli, embryos producing embryogenic callus were subcultured onto a selection medium (E3Select) consisting of 2× Murashige and Skoog (1962) macronutrients and 1× micronutrients and organic vitamins, 40 mg/L thiamine, 150 mg/L L-asparagine, supplemented with 2% (w/v) sucrose, 0.8% (w/v) Sigma-agar, 5 mg/L of D,L phosphinothricin (PPT) and no plant growth regulators. Cultures were incubated for further 14 days on E3Select at 24° C. in the light and a 12-hour photoperiod. This step is shown in FIGS. 14e,14f.

Step 5 (Day 30):

After 14 days culture on E3Select, embryogenic calli were sub-cultured onto fresh E3Select for a further 14 days.

Step 6 (Day 44):

After about 4 weeks on E3Select, developing plantlets (FIGS. 14g, 14h) were excised from the embryonic callus mass and grown for a further three weeks in 65 mm×80 mm or 65 mm×150 mm polycarbonate tissue culture vessels containing root induction medium (RM) as shown in FIG. 14i. Root induction medium consists of 1× Murashige and Skoog (1962) macronutrients, micronutrients and organic vitamins, 40 mg/L thiamine, 150 mg/L L-asparagine, supplemented with 2% (w/v) sucrose, 0.8% (w/v) Sigma-agar, and 5 mg/L of PPT. Remaining embryogenic callus is sub-cultured onto E3Select for another 14 days.

Step 7 (Day 65+):

Regenerated plantlets surviving greater than 3 weeks on root induction medium with healthy root formation were potted into a nursery mix consisting of peat and sand (1:1) and kept at 22-24° C. with elevated humidity under a nursery humidity chamber system (FIG. 14h). After two weeks, plants were removed from the humidity chamber and hand watered and liquid fed Aquasol™ weekly until maturity. The T₀ plants were sampled for genomic DNA and molecular analysis. T1 seeds are collected and planted for high-throughput Q-PCR analysis.

c) Agrobacterium-Mediated Transformation of Arabidopsis thaliana

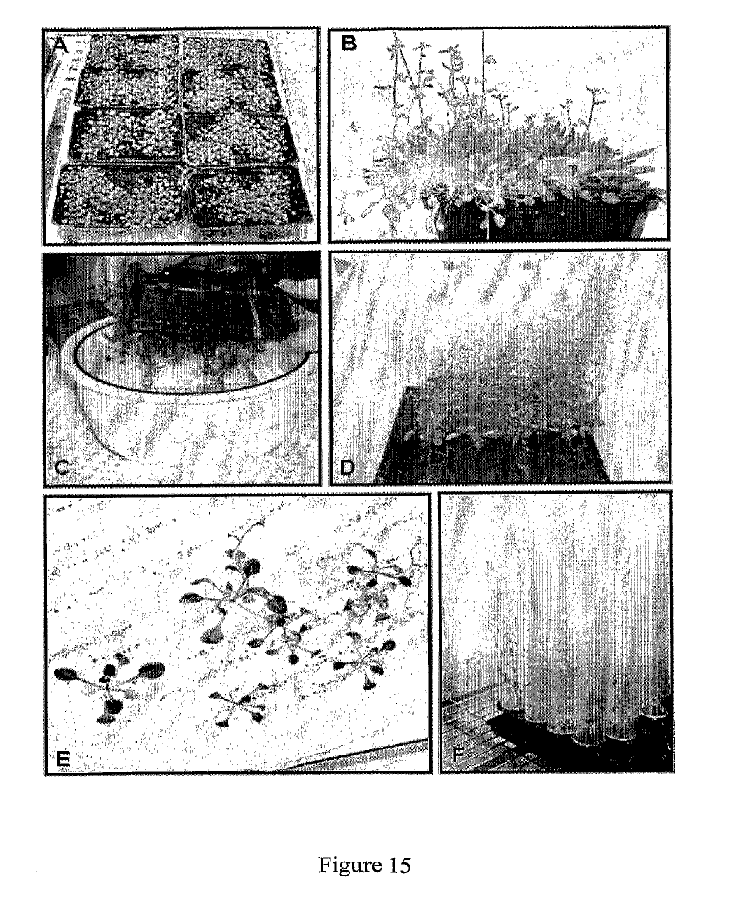

Binary vectors described herein above are transformed into the Agrobacterium tumefaciens strain AGL1 and in planta transformation of Arabidopsis thaliana is performed via vacuum infiltration of floral tissues. Briefly, a container (500 or 1,000 mL capacity) is placed inside a vacuum dessicator and filled with bacterial suspension. A punnet containing approximately 4-week-old Arabidopsis plants is inverted and immersed in the bacterial suspension, including rosette leaves. The lid of the dessicator was attached and vacuum applied until the gauge read approximately 250 mm (10 inches) Hg. Plants are left under vacuum for two minutes. Plants are then removed and excess bacterial suspension is allowed to drain from the plants. The plants are returned to the growth room, covered with a dome or plastic wrap and kept away from direct light overnight. The following day plants are returned to direct light and the dome or plastic wrap is removed. Plants are allowed to grow until the siliques are fully developed and dry seed is harvested. Arabidopsis seed is surface-sterilised and plated on selective media and putative transgenic Arabidopsis plants transferred to soil for the recovery of T₂ transgenic seed. These steps are shown in FIG. 15.

### d) Agrobacterium-Mediated Transformation of Maize

The transformation of maize is performed using, for example, a technique described in International Patent Publication No. WO 2006/136596 A2 and/or WO 2007/014744 A2.

Step 1: Preparation of Agrobacterium

Briefly, inoculums of Agrobacterium were streaked from glycerol stocks onto YP agar medium containing appropriate antibiotics (e.g. 50 mg/L spectinomycin and/or 10 mg/L tetracycline). The bacterial cultures are incubated in the dark at 28° C. for 1 to 3 days, or until single colonies are visible. The obtained plate is stored at 4° C. for 1 month and used as a master plate to streak out fresh cells. Fresh cells are streaked onto YP agar with the appropriate antibiotic from a single colony on the master plate, at least 2 days in advance of transformation. These bacterial cultures are incubated in the dark at 28° C. for 1 to 3 days.

Alternatively a frozen Agrobacterium stock is prepared by streaking Agrobacterium cells from a frozen stock onto a plate B-YP-002 (YP+50 mg/L spectinomycin+10 mg/L tetracycline), and grown at 28° C. for 2 to 3 days. A master plate is produced and stored at 4° C. for up to a month. From the master plate, cells are picked and added to a flask containing 25 ml liquid B-YP-000 medium supplemented with 50 mg/L Spectinomycin+10 mg/L tetracycline. The flask is incubated at 28° C. on a shaker set at 300 rpm for 2 to 3 days. A frozen Agrobacterium stock is prepared by mixing 1 part of the resulting culture with 1 part of sterile 30% glycerol. The mixture is then vortexed to mix well and 10 μl of the Agrobacterium/glycerol mixture dispensed to an Eppendorf tube. This stock is stored at −80° C.

To prepare cells for infection, cells from the bacterial culture described in the previous paragraphs are suspended in 1.0 to 1.8 mL LS-inf medium supplemented with 100 μM acetosyringone. This yields a bacterial suspension with approximate optical density (OD600) between 0.5 and 2.0. The mixture is vortexed for 0.5 to 3 hours. Approximately 100 μL of the Agrobacterium cell suspension is mixed with 900 μL of LS-inf solution in a cuvette, and the optical density (OD600) is measured. The optical density (OD600) of the Agrobacterium solution is adjusted to between about 0.6 and about 2.0 with LS-Inf (with 100 μM acetosyringone) solution. This Agrobacterium suspension is vortexed in the LS-inf+acetosyringone media for at least 0.5 to 3 hours prior to infection.

Alternatively, Agrobacterium suspensions for maize transformation are prepared as follows, two days before transformation, Agrobacteria solution from a frozen stock is streaked onto a plate containing B-YP-002 (solidified YP+50 mg/L spectinomycin+10 mg/L tetracycline) and grown at 28° C. in the dark for two days. About 1 to 4 hrs before transformation, a sample of bacterial cells is added to 1.5 ml M-LS-002 medium (LSinf+200 μM acetosyringone) in a 2 ml Eppendorf tube and the sample vortexed at about 1000 rpm for 1 to 4 hrs. The OD600 of the resulting solution should be in the range of about 0.6 to about 1.0 or about 108 cfu/mL.

For the purpose of the following example maize are transformed with Agrobacterium tumefaciens strain LBA4404 or disarmed Agrobacterium strain K599 (NCPPB 2659) transformed with a binary vector containing an acetohydroxyacid synthase (ahas gene) (as a selectable marker) and a GUS reporter gene.

Step 2: Surface Sterilization of Maize Ear and Isolation of Immature Embryos

Maize ears are harvested from one or more plants in a greenhouse 8 to 12 days after pollination. All husk and silks are removed and ears are transported into a tissue culture laboratory. A large pair of forceps is inserted into the basal end of the ear and the forceps are used as a handle for handling the cob.

Optionally, when insects/fungus are present on the ear, the ear is sterilized with 20% commercial bleach for 10 min (alternatively 30% Clorox solution for 15 min), and then rinsed with sterilized water three times. While holding the cob by the forceps, the ear is completely sprayed with 70% ethanol and then rinsed with sterile ddH2O.

Step 3: Inoculation

Method 1: the Modified “Tube” Method

The cob with the forceps handle is placed in a large Petri plate. The top portion (approximately two thirds) of each kernel is removed, e.g., with a scalpel. The immature embryos are then excised from the kernels on the cob, e.g., with a scalpel. In this respect, the scalpel blade is inserted on an angle into one end of a kernel, and the endosperm is lifted upwards away from the embryo which is positioned under the endosperm. Excised embryos are collected in a microfuge tube (or a small Petri plate) containing roughly 1.5 to 1.8 mL of Agrobacterium suspension in LS-inf liquid medium containing acetosyringone. The tube containing embryos is hand-mixed several times, and the incubated at room temperature (20 to 25° C.) for 30 min. Excess bacterial suspension is removed from the tube/plate with a pipette. Immature embryos and bacteria are transferred in the residue LS-inf medium to a Petri plate containing co-cultivation agar medium. The immature embryos are placed on the co-cultivation medium with the flat side down (scutellum upward). The majority of the excess bacterial suspension is removed with a pipette. A small amount of liquid is left on the plate to avoid drying of the embryos while plating.

The plate cover is left open in a sterile hood for about 15 min to evaporate excess moisture covering immature embryos. Petri dishes are sealed and incubated in the dark at 22° C. for 2 to 3 days. A selection of immature embryos (e.g., three to five embryos) is removed for GUS staining if a GUS construct is used to assess transient GUS expression.

Method 2: the “Drop” Method

Excised immature embryos are directly placed onto co-cultivation medium with the flat side down (scutellum upward). Five microlitres of diluted Agrobacterium cell suspension is added each immature embryo. Excess moisture covering immature embryos is evaporated by leaving the plate cover open in the hood for about 15 min. The plate is then sealed and incubated in the dark at 22° C. for 2 to 3 days. A selection of immature embryos (e.g., three to five embryos) is then analysed for GUS staining if a GUS construct is used to assess transient GUS expression.

### Step 4: Recovery

After co-cultivation, the embryos are transferred to recovery media and incubated in the dark at 27° C. for about 5 to 10 days, with the scutellum side up.

### Step 5: Selection

Immature embryos are transferred to first selection media. Petri plates are sealed and incubated in the dark at 27° C. for 10 to 14 days (First selection). All immature embryos that produce variable calli are subcultured into second selection media. At this stage, any shoots that have formed are removed. Plates are then sealed and incubated in the dark at 27° C. for about 2 weeks under the same conditions for the first selection. Regenerable calli are then excised from the scutellum under a stereoscopic microscope. Calli are transferred to fresh the 2nd selection media, sealed and incubated in the dark at 27° C. for 2 weeks.

Step 6: Regeneration and Transplanting of Transformed Plants

Proliferating calli are excised in the same manner as for second selection and transferred to regeneration media in 25×100 mm plates. Plates are sealed and placed under light (ca. 2,000 lux; 14/10 hr light/dark) at 25° C. or 27° C. for two to three weeks, or until shoot-like structures are visible.

Calli sections with regenerated shoots or shoot-like structures are transferred to a Phytatray or Magenta box containing rooting medium and incubated for 2 weeks under the same conditions discussed in the previous paragraph, or until rooted plantlets have developed. After 2 to 4 weeks on rooting media, calli that still have green regions are transferred to fresh rooting Phytatrays. Seedling samples are taken for TaqMan analysis to determine the number of transfer DNA (T-DNA) insertions.

Rooted seedlings are then transferred to Metromix soil in greenhouse and covered with a plastic dome until seedlings have established, which is generally about one week. Plants are maintained with daily watering, and liquid fertilizer twice a week. When plants reach the 3 to 4-leaf stage, they are fertilized with Osmocote™. If needed, putative transgenic plants are sprayed with 70 to 100 g/ha Pursuit™, and grown in the greenhouse for another two weeks. Non-transgenic plants generally develop herbicidal symptoms or die within this time. Surviving plants are transplanted into 10 inch pots with Metromix and 1 teaspoon Osmocote™.

At the flowering stage, tassels of transgenic plants are bagged with brown paper bags to prevent pollen escape. Pollination is performed on the transgenic plants. If silking and anthesis are not synchronized, a wild-type pollen donor or recipient plant with same genetic background as the transgenic T₀ plant is used for cross-pollination. T₁ seeds are harvested, dried and stored properly with adequate label on the seed bag. After harvesting the transgenic T₁ seeds, T₀ plants including the soil and pot may be sterilized by heat-treatment in an autoclave.

Using such a procedure, the binary vectors pRHF112 and pRHF121 were used to produce transformed maize.

### 2. Plant Transformation Results

### a) Expression of Reporter Gene in Wheat Under Control of WP05 and WP07

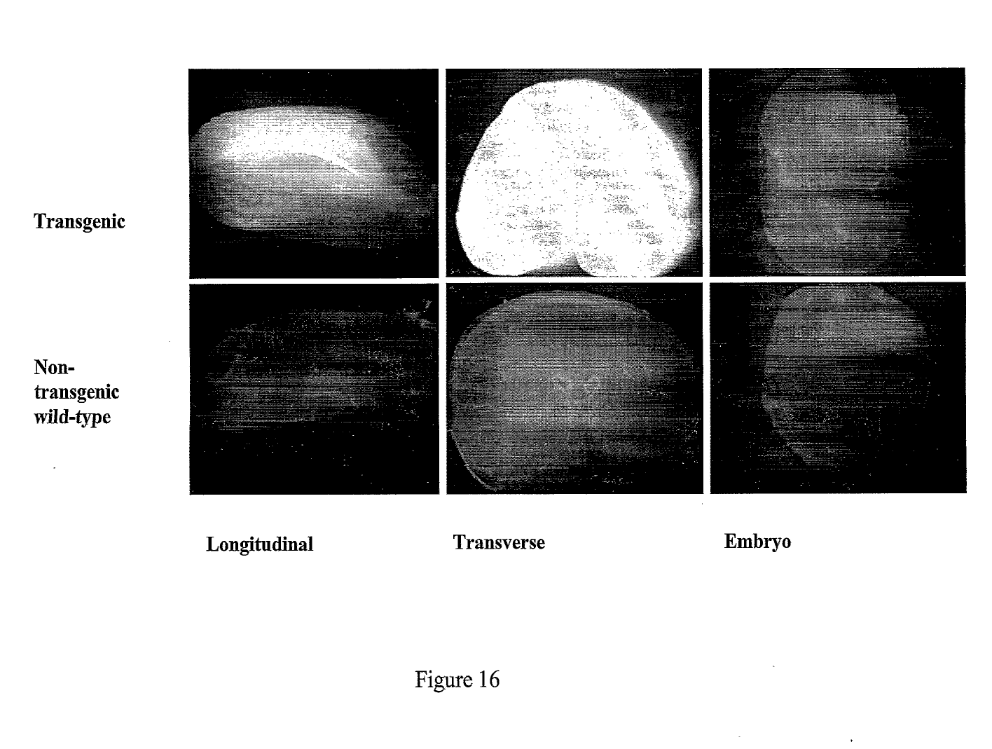

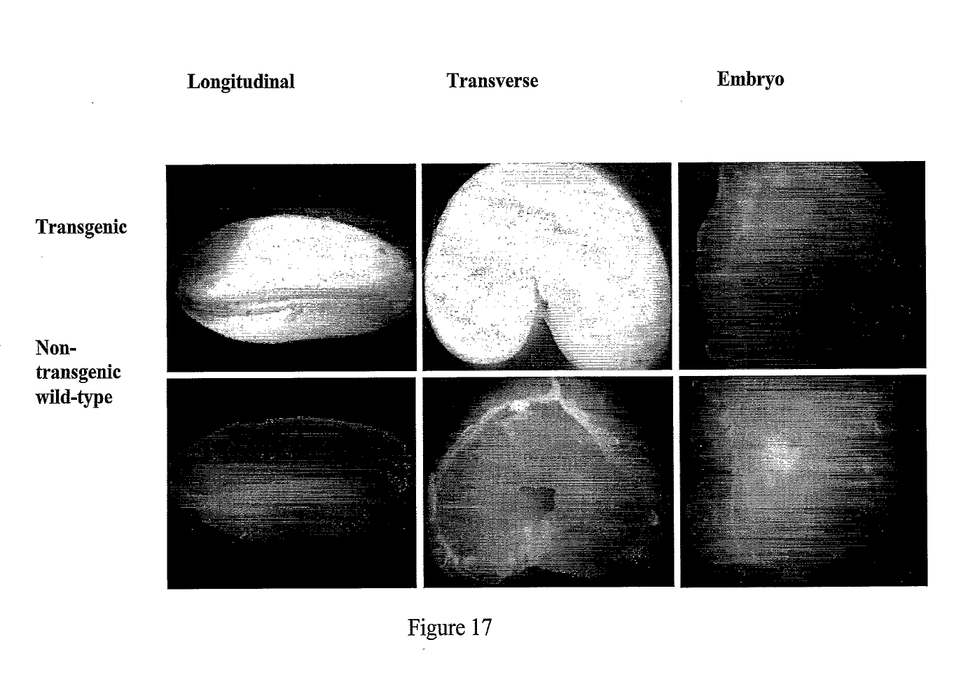

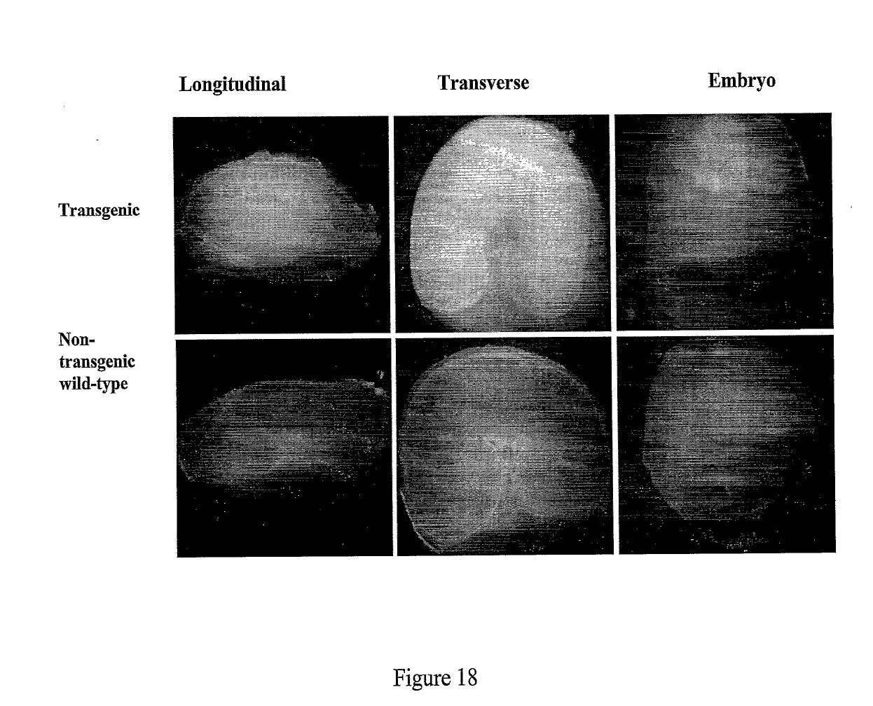

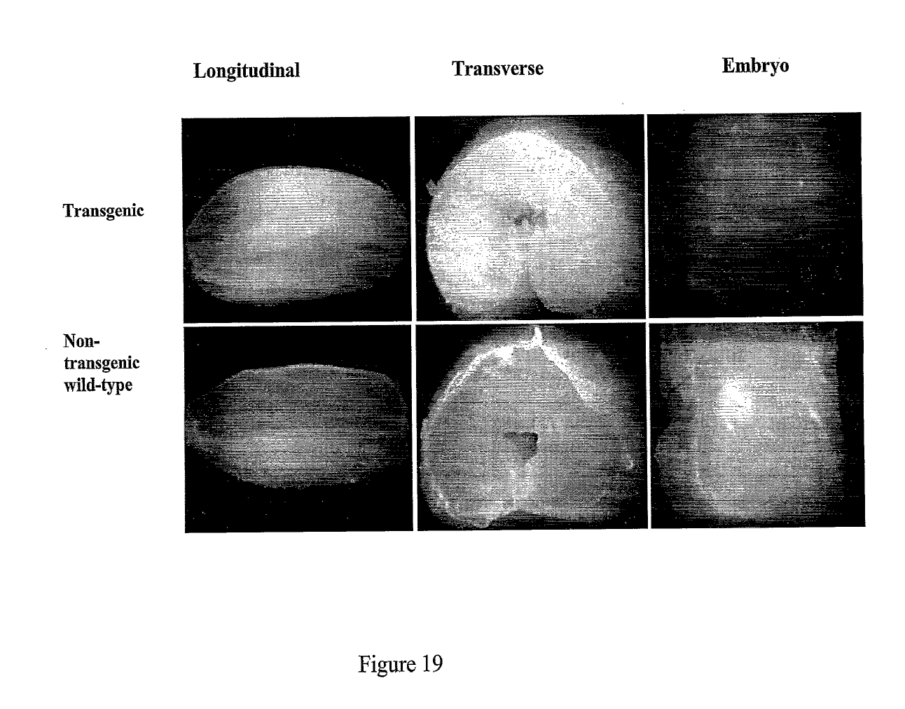

The WP05::sgfp-nos and WP07::sgfp-nos transformation vectors were used for biolistic transformation of wheat (Triticum aestivum L. MPS Bobwhite 26) and the resulting transgenics were sectioned and analysed for presence of GFP to demonstrate the spatial expression of the wheat promoters (FIGS. 16-19). Expression of GFP under control of both the WP05 and WP07 promoters was detected predominantly in the endosperm of the developing seed about ten days after pollination (DAP) and continuing to about 30 DAP (FIGS. 47-50). This corresponds to the period of grain filling. No expression was evident in vegetative organs e.g., leaves, root, stem node, stem internode or glumes, or in the reproductive tissues e.g., anthers, ovaries or pollen, or in mature seed (data not shown). These data indicate that the WP05 promoter and WP07 promoter both confer endosperm-selective expression, and most likely strictly endosperm-specific expression, on a gene to which the promoter is operably connected in developing seeds of wheat.

b) Expression of Reporter Gene in Maize Under Control of WP05 and WP07

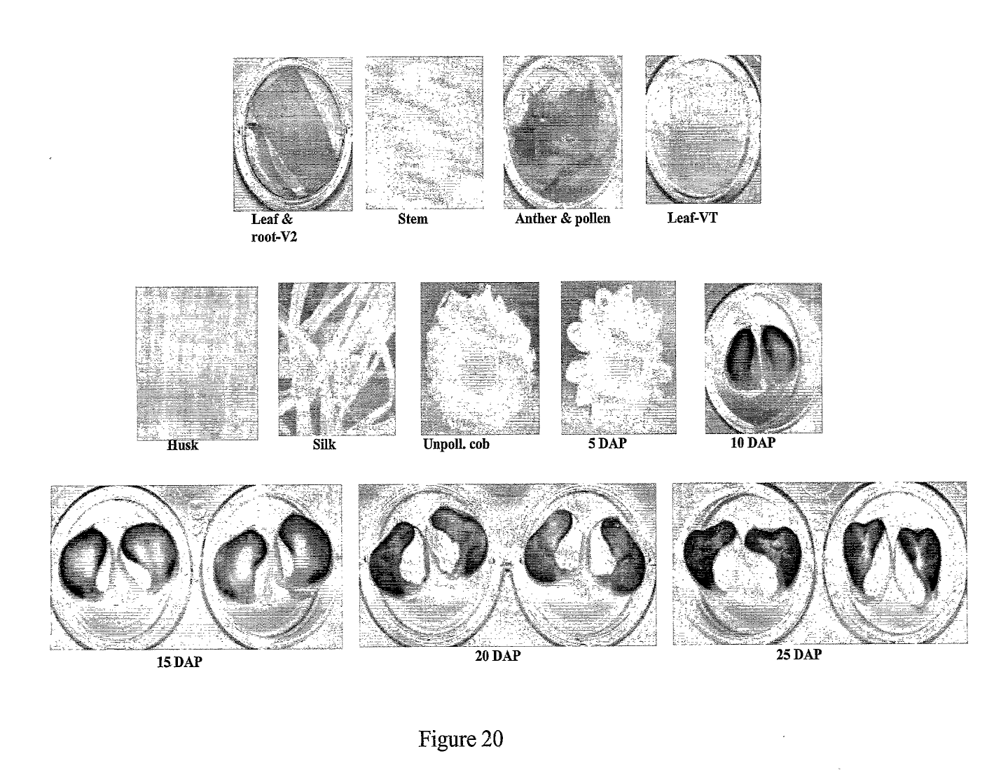

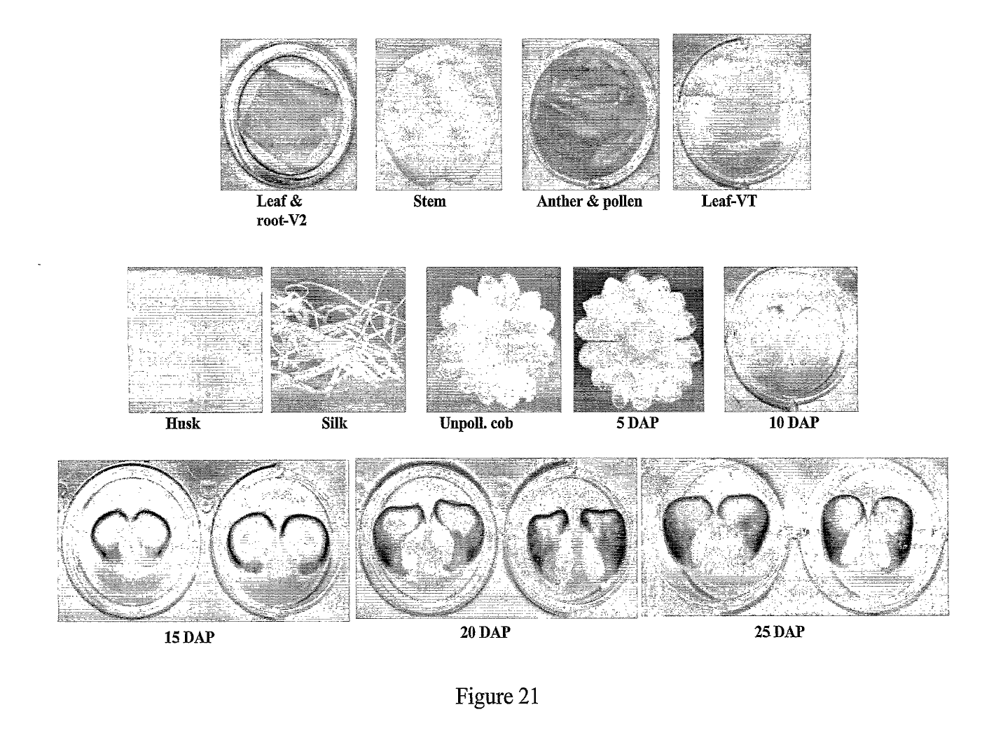

The binary vectors RHF112 (FIG. 11; SEQ ID NO: 18) and RHF121 (FIG. 12; SEQ ID NO: 19), each comprising a GUS expression cassette driven by the wheat WP05 promoter (vector RHF112) or wheat WP07 promoter (vector RHF121) was used to transform maize plants. The resulting transgenics were sectioned and analyzed for GUS expression. Data presented in FIGS. 20 and 21 demonstrate that expression of the GUS reporter gene under control of the WP05 promoter (FIG. 20) and the WP07 promoter (FIG. 21) is predominantly localized to the endosperm. The WP05 promoter conferred strong expression in the endosperm of maize, compared to the expression conferred by the WP07 promoter. Similar to the expression conferred in wheat by these promoters, GUS activity was observed 5-10 days after pollination (DAP) in maize endosperm, continuing throughout grain development to at least 25 DAP. Slightly earlier expression was detectable for the WP05 promoter than the WP07 promoter, possibly due to the stronger activity of the WP05 promoter in developing maize seeds. AS with expression in wheat, no reporter expression was apparent in vegetative organs e.g., leaves, root or stem, or in the reproductive tissues e.g., anthers, ovaries or pollen, or in husks or silks. These data indicate that the WP05 promoter and WP07 promoter both confer endosperm-selective expression, and most likely strictly endosperm-specific expression, on a gene to which the promoter is operably connected in developing seeds of maize.

### Example 4

### Characterization of WP05 and WP07 Equivalents from Monocots

This example provides support for a sub-genus of endosperm-selective promoters in monocotyledonous plants that are equivalents to the isolated wheat-derived promoters WP05 and/or WP07 e.g., by virtue of regulating genes that are structurally related to the genes that the WP05 and/or WP07 promoters control in their native contexts.

1. Equivalents of WP05 in maize, barley and rice

To identify equivalent promoters to WP05, the wheat Affymetrix Consensus Ta.10021.1.S1_at sequence was used as a BLASTN query against the NCBI non-redundant nucleotide database and a database of wheat assembled ESTs downloaded from the Plant Genome Database (http://www.plantgdb.org/). This approach identified two sequences in the GenBank non-redundant database, a wheat sequence assigned Accession No. BT008988.1 with 93% maximum identity to WP05, and a barley sequence Accession No. AK252536.1 with 87% maximum identity to WP05. A search of the wheat assembled ESTs also identified a sequence with 100% maximum identity assigned Accession No. PUT-153a-Triticum_{—}aestivum-124535. An alignment of Accession Nos. BT008988.1 and PUT-153a-Triticum_{—}aestivum-124535 to the Affymetrix Consensus Ta.10021.1.S1_at sequence and Genome Walker primer sequences CTTCAACGACCGCATACTGC and GAGGACGGCATGATGATC confirmed their relatedness (not shown).

The PUT-153a-Triticum_{—}aestivum-124535 sequence was used to search cDNA sequences extracted from the database of rice pseudomolecules produced by the TIGR Rice Genome Annotation Project (http://blast.jcvi.org/euk-blast/index.cgi) using the BLASTN algorithm with a nucleotide mismatch penalty (−q) of −1. A number of related sequences were identified, including Accession No. LOC_Os01 g01290.1, a histone like transcription factor. The positioning of LOC_Os01g01290.1 is viewable in the TIGR genome browser. The MPSS expression profile of the rice LOC_Os01g01290.1 indicates that this gene is expressed in 6 day old developing seed libraries in rice, e.g., consistent with the expression pattern for SEQ ID NO: 1 which is regulated in its native context by the WP05 promoter.

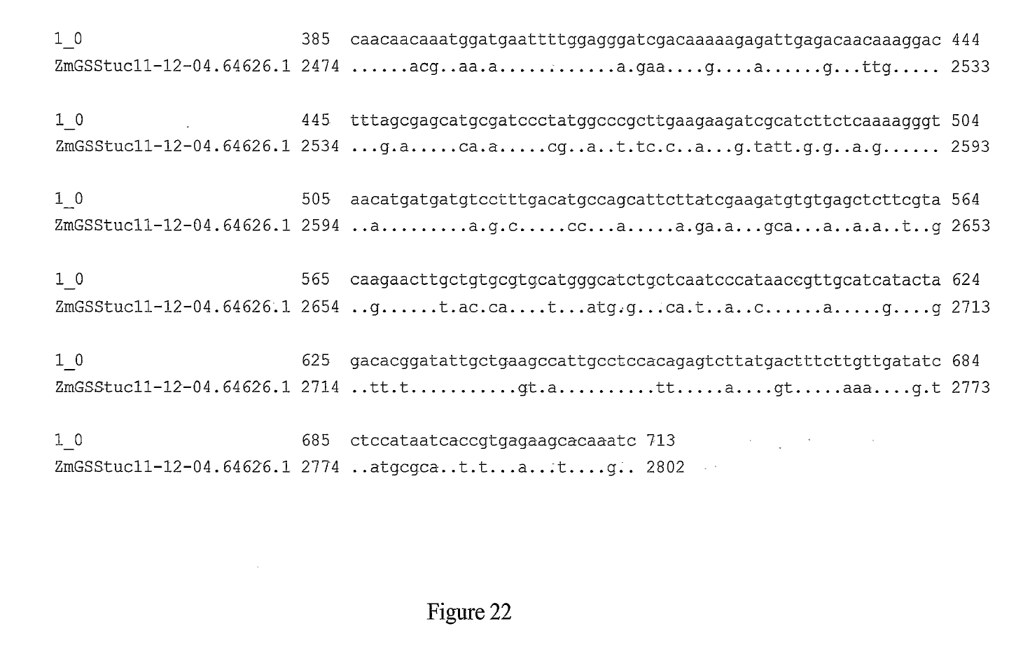

Contig assemblies of the maize genome assembled by the Plant Genome Database (http://www.plantgdb.org/) were downloaded and searched using the complete genomic sequence of LOC_Os01g01290.1 with a nucleotide mismatch penalty (−q) of −1. One maize genomic DNA assembly, assigned Accession No. ZmGSStuc11-12-04.64626 was identified having close sequence identity to residues 385-713 of LOC_Os01g01290.1 (FIG. 22). Multiple Alignment of Affymetrix Consensus Ta.10021.1.S1_at, Accession No. PUT-153a-Triticum_{—}aestivum-124535, Accession No. LOC_Os01 g01290.1, Accession No. ZmGSStuc11-12-04.64626 and another cDNA sequence (Accession No. PUT-153a-Triticum_{—}aestivum-124587) that showed similarity to the PUT-153a-Triticum_{—}aestivum-124535, permitted identification of a putative translation start codon (not shown). The 3′-end of the WP05 promoter sequence (SEQ ID NO: 3) aligned to these sequences upstream of this putative translation start codon.

These data suggest that Accession No. LOC_Os01 g01290.1, Accession No. ZmGSStuc11-12-04.64626, Accession No. PUT-153a-Triticum_{—}aestivum-124587 and Accession No. PUT-153a-Triticum_{—}aestivum-124535 comprise equivalents, e.g., functional and/or structural equivalents, to the WP05 promoter exemplified herein. The sequence of the 5′-upstream region of LOC_Os01 g01290.1 is presented in SEQ ID NO: 6. The sequence of the 5′-upstream region of ZmGSStuc1′-12-04.64626 is presented in SEQ ID NO: 7.

### 2. Equivalents of WP07 in Maize, Sorghum and Rice

To identify equivalent promoters to WP07, the wheat cDNA (SEQ ID NO: 2) was used as a BLASTN query against the GenBank non-redundant nucleotide database. This approach identified eight sequences (Table 3).

TABLE 3

| Accession No. | Description | Max_identity |
|---|---|---|
| AB085212.1 | Wheat Tria27 | 93% |
| CT831595.1 | Indica rice cDNA clone: | 87% |
| | OSIGCSA059P08 | |
| AK106050.1 | Japonica rice cDNA clone: | 87% |
| | 001-206-F01 | |
| CT832278.1 | Indica rice cDNA clone: | 90% |
| | OSIGCRA121J01 | |
| NM_001056362.1 | Japonica rice clone: | 87% |
| | Os03g0295800 | |
| AK071633.1 | Japonica rice clone: J023102J23 | 87% |
| DQ244863.1 | Zea mays clone 11235 | 85% |
| | mRNA sequence | |
| AC118670.2 | Nipponbare rice clone: | 94% |
| | OSJNBb0036D03 | |

The rice cDNA clone OSIGCSA059P08a (GenBank Accession No. CT831595.1) was used to search gene sequences extracted from the database of rice pseudomolecules produced by the TIGR Rice Genome Annotation Project (http://blast.jcvi.org/euk-blast/index.cgi) using the BLASTN algorithm. This approach identified Accession No. LOC_Os03g18454. The structure of LOC_Os03g18454 suggests two transcripts that are alternatively spliced (not shown) wherein exon 1 of transcript 2 is similar to exon 1 of transcript 1. The MPSS expression profile of the rice LOC_Os03g18454 indicates that this gene is expressed in 6 day old developing seed libraries in rice, e.g., consistent with the expression pattern for SEQ ID NO: 1 which is regulated in its native context by the WP07 promoter.

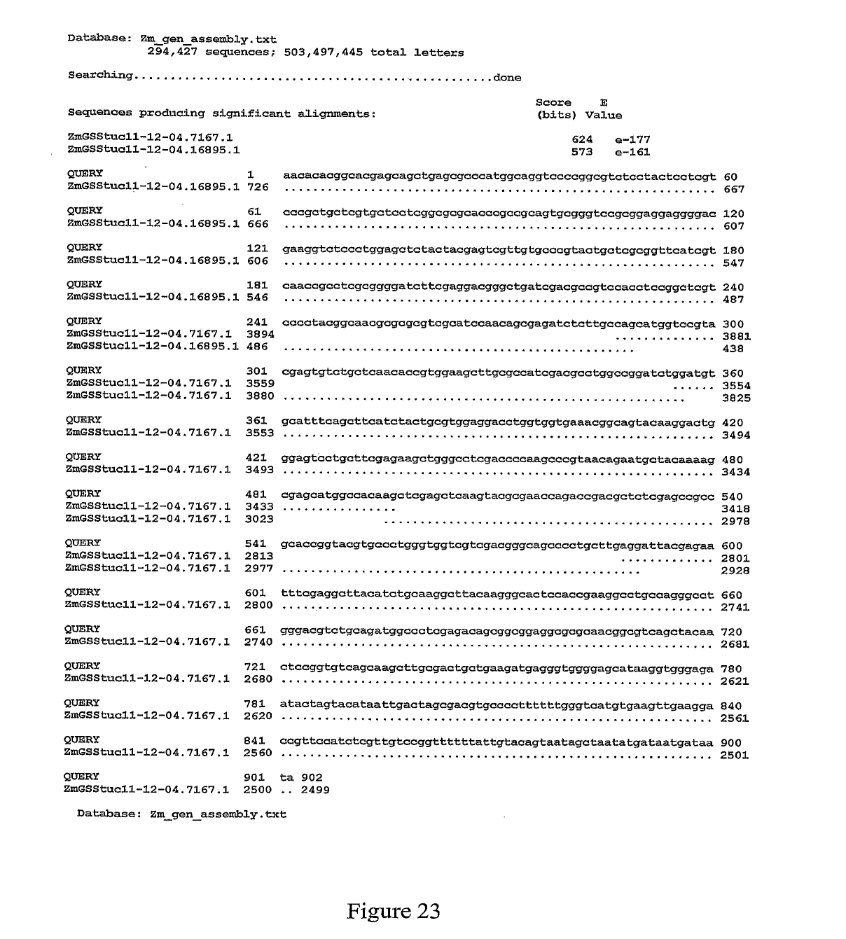

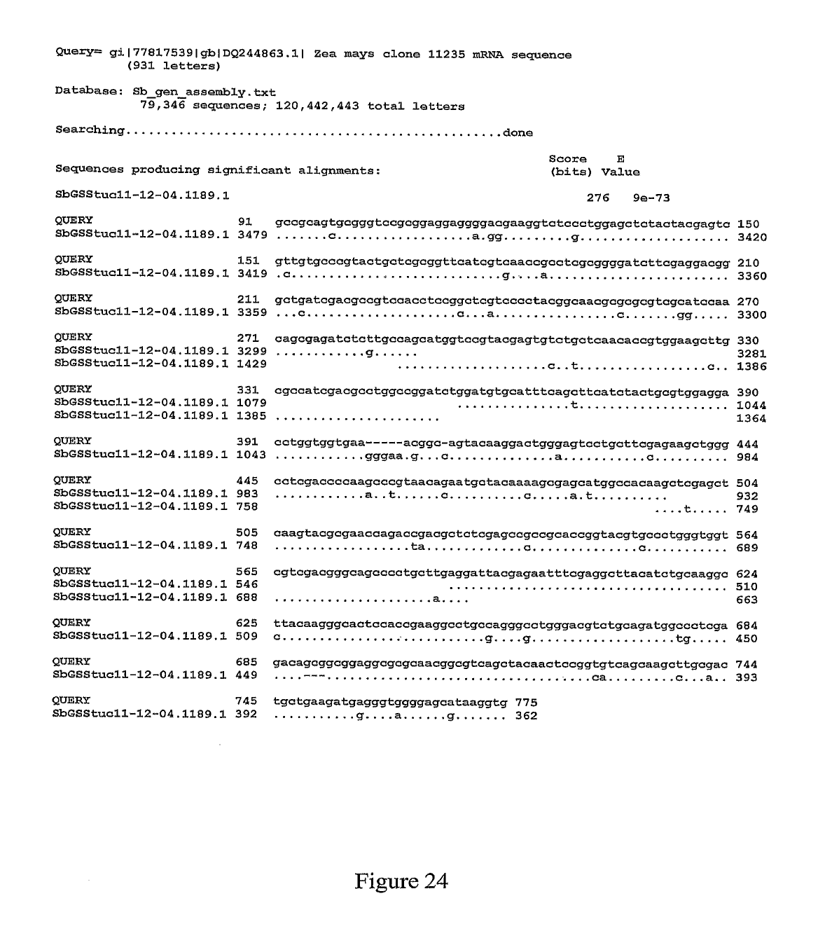

Contig assemblies of the sorghum and maize genomes assembled by the Plant Genome Database (http://www.plantgdb.org/) were downloaded and searched using the cDNA sequence of GenBank Accession No. DQ244863.1. Two non-overlapping maize genomic DNA assemblies, assigned Accession Nos. ZmGSStuc11-12-04.7167.1 and ZmGSStuc11-12-04.16895.1 (FIG. 23), and one sorghum DNA assembly assigned Accession No. SbGSStuc11-12-04.1189.1 (FIG. 24), were identified having close sequence identity to the query sequence. Multiple Alignment of SEQ ID NO: 2 and maize and sorghum genomic sequences comprising the sequences set forth in FIGS. 23 and 24, and other cDNAs of wheat having identity to SEQ ID NO: 2 (e.g., Table 3), permitted identification of a putative translation start codon (not shown).

These data suggest that Accession No. LOC_Os03g18454, Accession No. ZmGSStuc11-12-04.7167.1, Accession No. ZmGSStuc11-12-04.16895.1 and Accession No. SbGSStuc11-12-04.1189.1 comprise equivalents, e.g., functional and/or structural equivalents, to the WP07 promoter exemplified herein. The sequence of the 5′-upstream region of ZmGSStuc11-12-04.16895.1 is presented in SEQ ID NOs: 8 and 9.

### Example 5

### Structural Analysis of Promoters

This example provides support for structural conservation between the functional endosperm promoters WP05 (SEQ ID NO: 3) and WP07 (SEQ ID NO: 4) and the 5′-upstream sequences of Accession No. LOC_Os01 g01290.1 (SEQ ID NO: 6), Accession No. ZmGSStuc11-12-04.64626 (SEQ ID NO: 7) and Accession No. ZmGSStuc11-12-04.16895.1 (SEQ ID NO: 8).

Briefly, the nucleotide sequences of the wheat promoters were analyzed to determine cis-acting elements in the promoters, using PLACE (Plant cis-acting DNA elements) as described in Higo et al., Nucl. Acids Res. 27: 297-300, 1999, and available from National Institute of Agrobiological Sciences, Ibaraki, Japan. The results of this analysis are set forth in Tables 4-8.

TABLE 4
PLACE analysis results of the WP05 (1279 bp) promoter

| SITE_NAME | POSITION | STRAND | CONSENSUS |
|---|---|---|---|
| -300ELEMENT | 106 | (+) | TGHAAARK |
| -300ELEMENT | 254 | (−) | TGHAAARK |
| 2SSEEDPROTBANAPA | 1283 | (+) | CAAACAC |
| ABRELATERD1 | 1023 | (+) | ACGTG |
| ABRELATERD1 | 1270 | (+) | ACGTG |
| ABRELATERD1 | 775 | (−) | ACGTG |
| ABRERATCAL | 774 | (−) | MACGYGB |
| ABRERATCAL | 980 | (−) | MACGYGB |
| ACGTATERD1 | 776 | (+) | ACGT |
| ACGTATERD1 | 1023 | (+) | ACGT |
| ACGTATERD1 | 1270 | (+) | ACGT |
| ACGTATERD1 | 776 | (−) | ACGT |
| ACGTATERD1 | 1023 | (−) | ACGT |
| ACGTATERD1 | 1270 | (−) | ACGT |
| ACGTOSGLUB1 | 1268 | (+) | GTACGTG |
| ANAERO2CONSENSUS | 761 | (−) | AGCAGC |
| ANAERO2CONSENSUS | 1027 | (−) | AGCAGC |
| ARFAT | 419 | (+) | TGTCTC |
| ARR1AT | 72 | (+) | NGATT |
| ARR1AT | 936 | (+) | NGATT |
| ARR1AT | 1239 | (+) | NGATT |
| ARR1AT | 1258 | (+) | NGATT |
| ARR1AT | 286 | (+) | NGATT |
| ARR1AT | 414 | (+) | NGATT |
| ARR1AT | 111 | (−) | NGATT |
| BIHD1OS | 183 | (+) | TGTCA |
| BIHD1OS | 387 | (+) | TGTCA |
| BOXIINTPATPB | 10 | (+) | ATAGAA |
| BOXIINTPATPB | 363 | (+) | ATAGAA |
| BOXIINTPATPB | 468 | (+) | ATAGAA |
| BOXLCOREDCPAL | 514 | (+) | ACCWWCC |
| BOXLCOREDCPAL | 1110 | (+) | ACCWWCC |
| BOXLCOREDCPAL | 233 | (−) | ACCWWCC |
| BP5OSWX | 775 | (−) | CAACGTG |
| CAATBOX1 | 141 | (+) | CAAT |
| CAATBOX1 | 174 | (+) | CAAT |
| CAATBOX1 | 186 | (+) | CAAT |
| CAATBOX1 | 252 | (+) | CAAT |
| CAATBOX1 | 1011 | (+) | CAAT |
| CAATBOX1 | 53 | (−) | CAAT |
| CAATBOX1 | 74 | (−) | CAAT |
| CAATBOX1 | 88 | (−) | CAAT |
| CAATBOX1 | 288 | (−) | CAAT |
| CAATBOX1 | 1241 | (−) | CAAT |
| CACTFTPPCA1 | 260 | (+) | YACT |
| CACTFTPPCA1 | 331 | (+) | YACT |
| CACTFTPPCA1 | 357 | (+) | YACT |
| CACTFTPPCA1 | 390 | (+) | YACT |
| CACTFTPPCA1 | 1117 | (+) | YACT |
| CACTFTPPCA1 | 272 | (+) | YACT |
| CACTFTPPCA1 | 311 | (+) | YACT |
| CACTFTPPCA1 | 327 | (+) | YACT |
| CACTFTPPCA1 | 1058 | (+) | YACT |
| CACTFTPPCA1 | 1231 | (+) | YACT |
| CACTFTPPCA1 | 116 | (−) | YACT |
| CACTFTPPCA1 | 153 | (−) | YACT |
| CACTFTPPCA1 | 527 | (−) | YACT |
| CACTFTPPCA1 | 630 | (−) | YACT |
| CACTFTPPCA1 | 771 | (−) | YACT |
| CACTFTPPCA1 | 1200 | (−) | YACT |
| CANBNNAPA | 1283 | (+) | CNAACAC |
| CARGCW8GAT | 1082 | (+) | CWWWWWWWWG |
| CARGCW8GAT | 1154 | (+) | CWWWWWWWWG |
| CARGCW8GAT | 1082 | (−) | CWWWWWWWWG |
| CARGCW8GAT | 1154 | (−) | CWWWWWWWWG |
| CARGNCAT | 1081 | (+) | CCWWWWWWWWGG |
| CARGNCAT | 1081 | (−) | CCWWWWWWWWGG |
| CATATGGMSAUR | 179 | (+) | CATATG |
| CATATGGMSAUR | 179 | (−) | CATATG |
| CCAATBOX1 | 140 | (+) | CCAAT |
| CCAATBOX1 | 74 | (−) | CCAAT |
| CDA1ATCAB2 | 1289 | (+) | CAAAACGC |
| CGACGOSAMY3 | 711 | (−) | CGACG |
| CGCGBOXAT | 978 | (+) | VCGCGB |
| CGCGBOXAT | 980 | (+) | VCGCGB |
| CGCGBOXAT | 978 | (−) | VCGCGB |
| CGCGBOXAT | 980 | (−) | VCGCGB |
| CIACADIANLELHC | 379 | (−) | CAANNNNATC |
| CMSRE1IBSPOA | 632 | (+) | TGGACGG |
| CTRMCAMV35S | 66 | (−) | TCTCTCTCT |
| CURECORECR | 512 | (+) | GTAC |
| CURECORECR | 772 | (+) | GTAC |
| CURECORECR | 1268 | (+) | GTAC |
| CURECORECR | 1311 | (+) | GTAC |
| CURECORECR | 512 | (−) | GTAC |
| CURECORECR | 772 | (−) | GTAC |
| CURECORECR | 1268 | (−) | GTAC |
| CURECORECR | 1311 | (−) | GTAC |
| DOFCOREZM | 15 | (+) | AAAG |
| DOFCOREZM | 29 | (+) | AAAG |
| DOFCOREZM | 40 | (+) | AAAG |
| DOFCOREZM | 156 | (+) | AAAG |
| DOFCOREZM | 161 | (+) | AAAG |
| DOFCOREZM | 318 | (+) | AAAG |
| DOFCOREZM | 408 | (+) | AAAG |
| DOFCOREZM | 432 | (+) | AAAG |
| DOFCOREZM | 446 | (+) | AAAG |
| DOFCOREZM | 622 | (+) | AAAG |
| DOFCOREZM | 652 | (+) | AAAG |
| DOFCOREZM | 993 | (+) | AAAG |
| DOFCOREZM | 1007 | (+) | AAAG |
| DOFCOREZM | 1088 | (+) | AAAG |
| DOFCOREZM | 1318 | (+) | AAAG |
| DOFCOREZM | 4 | (−) | AAAG |
| DOFCOREZM | 262 | (−) | AAAG |
| DOFCOREZM | 333 | (−) | AAAG |
| DOFCOREZM | 929 | (−) | AAAG |
| DPBFCOREDCDC3 | 737 | (+) | ACACNNG |
| DPBFCOREDCDC3 | 948 | (+) | ACACNNG |
| DPBFCOREDCDC3 | 1045 | (+) | ACACNNG |
| DPBFCOREDCDC3 | 877 | (−) | ACACNNG |
| DPBFCOREDCDC3 | 1101 | (−) | ACACNNG |
| E2FCONSENSUS | 403 | (−) | WTTSSCSS |
| EBOXBNNAPA | 179 | (+) | CANNTG |
| EBOXBNNAPA | 225 | (+) | CANNTG |
| EBOXBNNAPA | 374 | (+) | CANNTG |
| EBOXBNNAPA | 451 | (+) | CANNTG |
| EBOXBNNAPA | 877 | (+) | CANNTG |
| EBOXBNNAPA | 179 | (−) | CANNTG |
| EBOXBNNAPA | 225 | (−) | CANNTG |
| EBOXBNNAPA | 374 | (−) | CANNTG |
| EBOXBNNAPA | 451 | (−) | CANNTG |
| EBOXBNNAPA | 877 | (−) | CANNTG |
| GATABOX | 379 | (+) | GATA |
| GATABOX | 467 | (+) | GATA |
| GATABOX | 803 | (+) | GATA |
| GATABOX | 1020 | (+) | GATA |
| GATABOX | 1229 | (+) | GATA |
| GATABOX | 144 | (−) | GATA |
| GATABOX | 239 | (−) | GATA |
| GATABOX | 569 | (−) | GATA |
| GATABOX | 675 | (−) | GATA |
| GATABOX | 1202 | (−) | GATA |
| GT1CONSENSUS | 366 | (+) | GRWAAW |
| GT1CONSENSUS | 604 | (+) | GRWAAW |
| GT1CONSENSUS | 605 | (+) | GRWAAW |
| GT1CONSENSUS | 255 | (−) | GRWAAW |
| GT1CONSENSUS | 298 | (−) | GRWAAW |
| GT1CONSENSUS | 567 | (−) | GRWAAW |
| GT1GMSCAM4 | 605 | (+) | GAAAAA |
| GT1GMSCAM4 | 255 | (−) | GAAAAA |
| GT1MOTIFPSRBCS | 296 | (−) | KWGTGRWAAWRW |
| GTGANTG10 | 117 | (+) | GTGA |
| GTGANTG10 | 348 | (+) | GTGA |
| GTGANTG10 | 863 | (+) | GTGA |
| GTGANTG10 | 902 | (+) | GTGA |
| GTGANTG10 | 259 | (−) | GTGA |
| GTGANTG10 | 330 | (−) | GTGA |
| GTGANTG10 | 356 | (−) | GTGA |
| GTGANTG10 | 389 | (−) | GTGA |
| GTGANTG10 | 479 | (−) | GTGA |
| GTGANTG10 | 600 | (−) | GTGA |
| GTGANTG10 | 733 | (−) | GTGA |
| IBOX | 803 | (+) | GATAAG |
| IBOXCORE | 803 | (+) | GATAA |
| IBOXCORE | 568 | (−) | GATAA |
| INRNTPSADB | 250 | (+) | YTCANTYY |
| INRNTPSADB | 329 | (+) | YTCANTYY |
| INRNTPSADB | 258 | (+) | YTCANTYY |
| INRNTPSADB | 1239 | (−) | YTCANTYY |
| MYB1AT | 537 | (+) | WAACCA |
| MYB2AT | 1250 | (+) | TAACTG |
| MYB2CONSENSUSAT | 1250 | (+) | YAACKG |
| MYBCORE | 1250 | (−) | CNGTTR |
| MYBCOREATCYCB1 | 957 | (−) | AACGG |
| MYBPZM | 233 | (−) | CCWACC |
| MYBST1 | 675 | (−) | GGATA |
| MYCCONSENSUSAT | 179 | (+) | CANNTG |
| MYCCONSENSUSAT | 225 | (+) | CANNTG |
| MYCCONSENSUSAT | 374 | (+) | CANNTG |
| MYCCONSENSUSAT | 451 | (+) | CANNTG |
| MYCCONSENSUSAT | 877 | (+) | CANNTG |
| MYCCONSENSUSAT | 179 | (−) | CANNTG |
| MYCCONSENSUSAT | 225 | (−) | CANNTG |
| MYCCONSENSUSAT | 374 | (−) | CANNTG |
| MYCCONSENSUSAT | 451 | (−) | CANNTG |
| MYCCONSENSUSAT | 877 | (−) | CANNTG |
| NODCON2GM | 92 | (+) | CTCTT |
| NODCON2GM | 392 | (+) | CTCTT |
| NODCON2GM | 701 | (+) | CTCTT |
| NODCON2GM | 65 | (−) | CTCTT |
| NTBBF1ARROLB | 261 | (+) | ACTTTA |
| NTBBF1ARROLB | 28 | (−) | ACTTTA |
| OSE2ROOTNODULE | 92 | (+) | CTCTT |
| OSE2ROOTNODULE | 392 | (+) | CTCTT |
| OSE2ROOTNODULE | 701 | (+) | CTCTT |
| OSE2ROOTNODULE | 65 | (−) | CTCTT |
| PALBOXAPC | 633 | (−) | CCGTCC |
| POLASIG1 | 36 | (+) | AATAAA |
| POLASIG1 | 50 | (−) | AATAAA |
| POLASIG2 | 940 | (−) | AATTAAA |
| POLLEN1LELAT52 | 12 | (+) | AGAAA |
| POLLEN1LELAT52 | 158 | (+) | AGAAA |
| POLLEN1LELAT52 | 365 | (+) | AGAAA |
| POLLEN1LELAT52 | 444 | (+) | AGAAA |
| POLLEN1LELAT52 | 470 | (+) | AGAAA |
| POLLEN1LELAT52 | 620 | (+) | AGAAA |
| POLLEN1LELAT52 | 716 | (−) | AGAAA |
| PREATPRODH | 1118 | (+) | ACTCAT |
| PRECONSCRHSP70A | 1103 | (−) | SCGAYNRNNNNNNNNNNNN |
| PYRIMIDINEBOXOSRAMY1A | 928 | (+) | CCTTTT |
| PYRIMIDINEBOXOSRAMY1A | 39 | (−) | CCTTTT |
| QARBNEXTA | 774 | (−) | AACGTGT |
| RAV1AAT | 1043 | (+) | CAACA |
| RAV1BAT | 225 | (−) | CACCTG |
| RAV1BAT | 877 | (−) | CACCTG |
| RBCSCONSENSUS | 412 | (−) | AATCCAA |
| REALPHALGLHCB21 | 1214 | (−) | AACCAA |
| RHERPATEXPA7 | 1023 | (−) | KCACGW |
| RHERPATEXPA7 | 1270 | (−) | KCACGW |
| ROOTMOTIFTAPOX1 | 142 | (−) | ATATT |
| ROOTMOTIFTAPOX1 | 187 | (−) | ATATT |
| ROOTMOTIFTAPOX1 | 1157 | (−) | ATATT |
| RYREPEATBNNAPA | 809 | (+) | CATGCA |
| RYREPEATBNNAPA | 821 | (+) | CATGCA |
| RYREPEATBNNAPA | 1279 | (+) | CATGCA |
| RYREPEATBNNAPA | 55 | (−) | CATGCA |
| RYREPEATBNNAPA | 505 | (−) | CATGCA |
| RYREPEATBNNAPA | 811 | (−) | CATGCA |
| RYREPEATBNNAPA | 819 | (−) | CATGCA |
| RYREPEATBNNAPA | 823 | (−) | CATGCA |
| RYREPEATBNNAPA | 1277 | (−) | CATGCA |
| RYREPEATGMGY2 | 809 | (+) | CATGCAT |
| RYREPEATGMGY2 | 821 | (+) | CATGCAT |
| RYREPEATGMGY2 | 810 | (−) | CATGCAT |
| RYREPEATGMGY2 | 822 | (−) | CATGCAT |
| RYREPEATLEGUMINBOX | 809 | (+) | CATGCAY |
| RYREPEATLEGUMINBOX | 821 | (+) | CATGCAY |
| RYREPEATLEGUMINBOX | 810 | (−) | CATGCAY |
| RYREPEATLEGUMINBOX | 822 | (−) | CATGCAY |
| RYREPEATLEGUMINBOX | 818 | (−) | CATGCAY |
| RYREPEATLEGUMINBOX | 1276 | (−) | CATGCAY |
| RYREPEATVFLEB4 | 809 | (+) | CATGCATG |
| RYREPEATVFLEB4 | 821 | (+) | CATGCATG |
| RYREPEATVFLEB4 | 809 | (−) | CATGCATG |
| RYREPEATVFLEB4 | 821 | (−) | CATGCATG |
| S1FBOXSORPS1L21 | 1265 | (+) | ATGGTA |
| SEBFCONSSTPR10A | 386 | (+) | YTGTCWC |
| SEBFCONSSTPR10A | 418 | (+) | YTGTCWC |
| SEF3MOTIFGM | 1127 | (+) | AACCCA |
| SEF4MOTIFGM7S | 305 | (−) | RTTTTTR |
| SITEIIATCYTC | 721 | (−) | TGGGCY |
| SORLIP1AT | 1092 | (+) | GCCAC |
| SORLIP1AT | 528 | (−) | GCCAC |
| SORLIP1AT | 912 | (−) | GCCAC |
| SORLIP1AT | 1300 | (−) | GCCAC |
| SORLIP2AT | 954 | (+) | GGGCC |
| SORLIP2AT | 721 | (−) | GGGCC |
| SURECOREATSULTR11 | 857 | (+) | GAGAC |
| SURECOREATSULTR11 | 420 | (−) | GAGAC |
| SV40COREENHAN | 536 | (−) | GTGGWWHG |
| T/GBOXATPIN2 | 775 | (−) | AACGTG |
| TAAAGSTKST1 | 28 | (+) | TAAAG |
| TAAAGSTKST1 | 155 | (+) | TAAAG |
| TAAAGSTKST1 | 431 | (+) | TAAAG |
| TAAAGSTKST1 | 1087 | (+) | TAAAG |
| TAAAGSTKST1 | 262 | (−) | TAAAG |
| TATABOX4 | 1083 | (+) | TATATAA |
| TATABOX5 | 35 | (−) | TTATTT |
| TATABOXOSPAL | 33 | (−) | TATTTAA |
| TATAPVTRNALEU | 1083 | (−) | TTTATATA |
| TBOXATGAPB | 3 | (+) | ACTTTG |
| TBOXATGAPB | 407 | (−) | ACTTTG |
| TRANSINITMONOCOTS | 696 | (−) | RMNAUGGC |
| WBOXATNPR1 | 184 | (−) | TTGAC |
| WBOXHVISO1 | 213 | (+) | TGACT |
| WBOXNTCHN48 | 212 | (+) | CTGACY |
| WBOXNTERF3 | 213 | (+) | TGACY |
| WRKY71OS | 213 | (+) | TGAC |
| WRKY71OS | 184 | (−) | TGAC |
| WRKY71OS | 388 | (−) | TGAC |

TABLE 5
PLACE analysis results of the WP07 (2400 bp) promoter

| SITE_NAME | POSITION | STRAND | CONSENSUS |
|---|---|---|---|
| -10PEHVPSBD | 473 | (+) | TATTCT |
| -300ELEMENT | 604 | (+) | TGHAAARK |
| -300ELEMENT | 666 | (+) | TGHAAARK |
| 2SSEEDPROTBANAPA | 842 | (+) | CAAACAC |
| 2SSEEDPROTBANAPA | 535 | (−) | CAAACAC |
| ABRELATERD1 | 588 | (−) | ACGTG |
| ACGTATERD1 | 133 | (+) | ACGT |
| ACGTATERD1 | 497 | (+) | ACGT |
| ACGTATERD1 | 589 | (+) | ACGT |
| ACGTATERD1 | 133 | (−) | ACGT |
| ACGTATERD1 | 497 | (−) | ACGT |
| ACGTATERD1 | 589 | (−) | ACGT |
| ACGTCBOX | 132 | (+) | GACGTC |
| ACGTCBOX | 132 | (−) | GACGTC |
| ANAERO2CONSENSUS | 16 | (+) | AGCAGC |
| ANAERO2CONSENSUS | 19 | (+) | AGCAGC |
| ANAERO2CONSENSUS | 102 | (+) | AGCAGC |
| ANAERO2CONSENSUS | 105 | (+) | AGCAGC |
| ANAERO2CONSENSUS | 108 | (+) | AGCAGC |
| ANAERO2CONSENSUS | 233 | (−) | AGCAGC |
| ANAERO2CONSENSUS | 354 | (−) | AGCAGC |
| ARR1AT | 466 | (+) | NGATT |
| ARR1AT | 361 | (+) | NGATT |
| ARR1AT | 563 | (−) | NGATT |
| ARR1AT | 609 | (−) | NGATT |
| ARR1AT | 671 | (−) | NGATT |
| ARR1AT | 860 | (−) | NGATT |
| ASF1MOTIFCAMV | 131 | (+) | TGACG |
| ASF1MOTIFCAMV | 498 | (−) | TGACG |
| ASF1MOTIFCAMV | 652 | (−) | TGACG |
| CAATBOX1 | 765 | (+) | CAAT |
| CAATBOX1 | 859 | (+) | CAAT |
| CACTFTPPCA1 | 442 | (+) | YACT |
| CACTFTPPCA1 | 772 | (+) | YACT |
| CACTFTPPCA1 | 252 | (+) | YACT |
| CACTFTPPCA1 | 370 | (+) | YACT |
| CACTFTPPCA1 | 479 | (+) | YACT |
| CACTFTPPCA1 | 791 | (−) | YACT |
| CANBNNAPA | 842 | (+) | CNAACAC |
| CANBNNAPA | 535 | (−) | CNAACAC |
| CATATGGMSAUR | 432 | (+) | CATATG |
| CATATGGMSAUR | 432 | (−) | CATATG |
| CBFHV | 177 | (+) | RYCGAC |
| CBFHV | 240 | (+) | RYCGAC |
| CBFHV | 63 | (−) | RYCGAC |
| CBFHV | 135 | (−) | RYCGAC |
| CCAATBOX1 | 764 | (+) | CCAAT |
| CGACGOSAMY3 | 155 | (+) | CGACG |
| CGACGOSAMY3 | 158 | (+) | CGACG |
| CGACGOSAMY3 | 179 | (+) | CGACG |
| CGACGOSAMY3 | 737 | (+) | CGACG |
| CGACGOSAMY3 | 62 | (−) | CGACG |
| CGACGOSAMY3 | 80 | (−) | CGACG |
| CGACGOSAMY3 | 134 | (−) | CGACG |
| CGACGOSAMY3 | 274 | (−) | CGACG |
| CGACGOSAMY3 | 277 | (−) | CGACG |
| CGCGBOXAT | 256 | (+) | VCGCGB |
| CGCGBOXAT | 1 | (+) | VCGCGB |
| CGCGBOXAT | 163 | (+) | VCGCGB |
| CGCGBOXAT | 256 | (−) | VCGCGB |
| CGCGBOXAT | 1 | (−) | VCGCGB |
| CGCGBOXAT | 163 | (−) | VCGCGB |
| CMSRE1IBSPOA | 725 | (−) | TGGACGG |
| CURECORECR | 89 | (+) | GTAC |
| CURECORECR | 484 | (+) | GTAC |
| CURECORECR | 89 | (−) | GTAC |
| CURECORECR | 484 | (−) | GTAC |
| DOFCOREZM | 801 | (+) | AAAG |
| DOFCOREZM | 805 | (+) | AAAG |
| DOFCOREZM | 358 | (−) | AAAG |
| DOFCOREZM | 558 | (−) | AAAG |
| DOFCOREZM | 635 | (−) | AAAG |
| DRE2COREZMRAB17 | 135 | (−) | ACCGAC |
| DRECRTCOREAT | 240 | (+) | RCCGAC |
| DRECRTCOREAT | 63 | (−) | RCCGAC |
| DRECRTCOREAT | 135 | (−) | RCCGAC |
| E2FCONSENSUS | 818 | (−) | WTTSSCSS |
| EBOXBNNAPA | 401 | (+) | CANNTG |
| EBOXBNNAPA | 432 | (+) | CANNTG |
| EBOXBNNAPA | 501 | (+) | CANNTG |
| EBOXBNNAPA | 600 | (+) | CANNTG |
| EBOXBNNAPA | 662 | (+) | CANNTG |
| EBOXBNNAPA | 401 | (−) | CANNTG |
| EBOXBNNAPA | 432 | (−) | CANNTG |
| EBOXBNNAPA | 501 | (−) | CANNTG |
| EBOXBNNAPA | 600 | (−) | CANNTG |
| EBOXBNNAPA | 662 | (−) | CANNTG |
| EECCRCAH1 | 703 | (−) | GANTTNC |
| GATABOX | 489 | (+) | GATA |
| GATABOX | 385 | (−) | GATA |
| GATABOX | 628 | (−) | GATA |
| GCCCORE | 119 | (−) | GCCGCC |
| GCCCORE | 147 | (−) | GCCGCC |
| GCCCORE | 150 | (−) | GCCGCC |
| GCCCORE | 167 | (−) | GCCGCC |
| GT1CONSENSUS | 489 | (+) | GRWAAW |
| GT1CONSENSUS | 605 | (+) | GRWAAW |
| GT1CONSENSUS | 667 | (+) | GRWAAW |
| GT1CONSENSUS | 798 | (+) | GRWAAW |
| GT1CONSENSUS | 821 | (+) | GRWAAW |
| GT1GMSCAM4 | 605 | (+) | GAAAAA |
| GT1GMSCAM4 | 667 | (+) | GAAAAA |
| GT1GMSCAM4 | 798 | (+) | GAAAAA |
| GTGANTG10 | 130 | (+) | GTGA |
| GTGANTG10 | 611 | (−) | GTGA |
| GTGANTG10 | 654 | (−) | GTGA |
| GTGANTG10 | 673 | (−) | GTGA |
| GTGANTG10 | 692 | (−) | GTGA |
| GTGANTG10 | 734 | (−) | GTGA |
| GTGANTG10 | 775 | (−) | GTGA |
| GTGANTG10 | 866 | (−) | GTGA |
| HEXAMERATH4 | 61 | (+) | CCGTCG |
| HEXAMERATH4 | 158 | (−) | CCGTCG |
| HEXAMERATH4 | 737 | (−) | CCGTCG |
| HEXMOTIFTAH3H4 | 497 | (+) | ACGTCA |
| HEXMOTIFTAH3H4 | 131 | (−) | ACGTCA |
| IBOXCORE | 489 | (+) | GATAA |
| IBOXCORE | 384 | (−) | GATAA |
| L1BOXATPDF1 | 547 | (−) | TAAATGYA |
| LTRE1HVBLT49 | 462 | (−) | CCGAAA |
| LTRECOREATCOR15 | 241 | (+) | CCGAC |
| LTRECOREATCOR15 | 63 | (−) | CCGAC |
| LTRECOREATCOR15 | 135 | (−) | CCGAC |
| LTRECOREATCOR15 | 278 | (−) | CCGAC |
| MYB1AT | 686 | (+) | WAACCA |
| MYB2AT | 366 | (−) | TAACTG |
| MYB2CONSENSUSAT | 366 | (−) | YAACKG |
| MYBCORE | 325 | (+) | CNGTTR |
| MYBCORE | 366 | (+) | CNGTTR |
| MYBCOREATCYCB1 | 743 | (+) | AACGG |
| MYBCOREATCYCB1 | 748 | (+) | AACGG |
| MYBST1 | 488 | (+) | GGATA |
| MYCCONSENSUSAT | 401 | (+) | CANNTG |
| MYCCONSENSUSAT | 432 | (+) | CANNTG |
| MYCCONSENSUSAT | 501 | (+) | CANNTG |
| MYCCONSENSUSAT | 600 | (+) | CANNTG |
| MYCCONSENSUSAT | 662 | (+) | CANNTG |
| MYCCONSENSUSAT | 401 | (−) | CANNTG |
| MYCCONSENSUSAT | 432 | (−) | CANNTG |
| MYCCONSENSUSAT | 501 | (−) | CANNTG |
| MYCCONSENSUSAT | 600 | (−) | CANNTG |
| MYCCONSENSUSAT | 662 | (−) | CANNTG |
| PALBOXAPC | 725 | (+) | CCGTCC |
| POLASIG1 | 516 | (−) | AATAAA |
| POLASIG3 | 468 | (−) | AATAAT |
| POLASIG3 | 471 | (−) | AATAAT |
| POLLEN1LELAT52 | 797 | (+) | AGAAA |
| POLLEN1LELAT52 | 803 | (+) | AGAAA |
| PRECONSCRHSP70A | 154 | (+) | SCGAYNRNNNNNNNNNN |
| | | | NNNNNHD |
| PRECONSCRHSP70A | 259 | (−) | SCGAYNRNNNNNNNNNN |
| | | | NNNNNHD |
| QELEMENTZMZM13 | 397 | (+) | AGGTCA |
| REBETALGLHCB21 | 487 | (+) | CGGATA |
| RHERPATEXPA7 | 587 | (+) | KCACGW |
| RHERPATEXPA7 | 852 | (+) | KCACGW |
| RHERPATEXPA7 | 734 | (+) | KCACGW |
| SITEIIATCYTC | 286 | (+) | TGGGCY |
| SITEIIATCYTC | 298 | (+) | TGGGCY |
| SORLIP1AT | 770 | (+) | GCCAC |
| SORLIP1AT | 139 | (−) | GCCAC |
| SORLIP2AT | 299 | (+) | GGGCC |
| SORLIP2AT | 678 | (+) | GGGCC |
| SORLIP2AT | 679 | (−) | GGGCC |
| SORLIP2AT | 722 | (−) | GGGCC |
| SREATMSD | 488 | (−) | TTATCC |
| SURECOREATSULTR11 | 204 | (+) | GAGAC |
| SURECOREATSULTR11 | 591 | (−) | GAGAC |
| TGACGTVMAMY | 131 | (+) | TGACGT |
| TGACGTVMAMY | 497 | (−) | TGACGT |
| UPRMOTIFIIAT | 764 | (+) | CCNNNNNNNNNNNNCCACG |
| WBOXHVISO1 | 314 | (+) | TGACT |
| WBOXHVISO1 | 393 | (−) | TGACT |
| WBOXHVISO1 | 787 | (−) | TGACT |
| WBOXNTCHN48 | 313 | (+) | CTGACY |
| WBOXNTCHN48 | 393 | (−) | CTGACY |
| WBOXNTCHN48 | 787 | (−) | CTGACY |
| WBOXNTCHN48 | 398 | (−) | CTGACY |
| WBOXNTERF3 | 314 | (+) | TGACY |
| WBOXNTERF3 | 393 | (−) | TGACY |
| WBOXNTERF3 | 787 | (−) | TGACY |
| WBOXNTERF3 | 398 | (−) | TGACY |
| WRKY71OS | 131 | (+) | TGAC |
| WRKY71OS | 314 | (+) | TGAC |
| WRKY71OS | 394 | (−) | TGAC |
| WRKY71OS | 399 | (−) | TGAC |
| WRKY71OS | 499 | (−) | TGAC |
| WRKY71OS | 653 | (−) | TGAC |
| WRKY71OS | 788 | (−) | TGAC |

TABLE 6
PLACE analysis results of the LOC_Os01g01290.1 upstream region

| SITE_NAME | POSITION (STRAND) | CONSENSUS |
|---|---|---|
| -10PEHVPSBD | 109 (+) | TATTCT |
| AMYBOX1 | 274 (+) | TAACARA |
| ARR1AT | 162 (+) | NGATT |
| ARR1AT | 181 (+) | NGATT |
| BIHD1OS | 8 (+) | TGTCA |
| BOXIINTPATPB | 135 (−) | ATAGAA |
| BOXLCOREDCPAL | 34 (+) | ACCWWCC |
| CAATBOX1 | 40 (+) | CAAT |
| CAATBOX1 | 222 (+) | CAAT |
| CAATBOX1 | 14 (−) | CAAT |
| CACTFTPPCA1 | 30 (+) | YACT |
| CACTFTPPCA1 | 98 (+) | YACT |
| CACTFTPPCA1 | 140 (+) | YACT |
| CACTFTPPCA1 | 146 (+) | YACT |
| CACTFTPPCA1 | 45 (−) | YACT |
| CACTFTPPCA1 | 82 (−) | YACT |
| CACTFTPPCA1 | 122 (−) | YACT |
| CACTFTPPCA1 | 259 (−) | YACT |
| CACTFTPPCA1 | 272 (−) | YACT |
| CACTFTPPCA1 | 281 (−) | YACT |
| CCAATBOX1 | 39 (+) | CCAAT |
| CPBCSPOR | 49 (−) | TATTAG |
| CURECORECR | 145 (+) | GTAC |
| CURECORECR | 145 (−) | GTAC |
| DOFCOREZM | 257 (+) | AAAG |
| DOFCOREZM | 267 (+) | AAAG |
| DOFCOREZM | 279 (+) | AAAG |
| DOFCOREZM | 320 (+) | AAAG |
| DOFCOREZM | 206 (−) | AAAG |
| DOFCOREZM | 244 (−) | AAAG |
| DPBFCOREDCDC3 | 4 (−) | ACACNNG |
| EBOXBNNAPA | 69 (+) | CANNTG |
| EBOXBNNAPA | 69 (−) | CANNTG |
| GAREAT | 274 (+) | TAACAAR |
| GATABOX | 264 (+) | GATA |
| GATABOX | 201 (−) | GATA |
| GT1CONSENSUS | 87 (+) | GRWAAW |
| GT1CONSENSUS | 153 (+) | GRWAAW |
| GT1CONSENSUS | 264 (+) | GRWAAW |
| GT1CONSENSUS | 61 (−) | GRWAAW |
| GT1CONSENSUS | 208 (−) | GRWAAW |
| GT1GMSCAM4 | 208 (−) | GAAAAA |
| GTGANTG10 | 151 (+) | GTGA |
| GTGANTG10 | 161 (+) | GTGA |
| IBOXCORE | 264 (+) | GATAA |
| MYB2CONSENSUSAT | 69 (−) | YAACKG |
| MYBCORE | 69 (+) | CNGTTR |
| MYBGAHV | 274 (+) | TAACAAA |
| MYBPZM | 35 (+) | CCWACC |
| MYCCONSENSUSAT | 69 (+) | CANNTG |
| MYCCONSENSUSAT | 69 (−) | CANNTG |
| NODCON2GM | 204 (+) | CTCTT |
| OSE2ROOTNODULE | 204 (+) | CTCTT |
| POLASIG3 | 107 (−) | AATAAT |
| POLASIG3 | 305 (−) | AATAAT |
| POLLEN1LELAT52 | 63 (−) | AGAAA |
| POLLEN1LELAT52 | 134 (−) | AGAAA |
| POLLEN1LELAT52 | 210 (−) | AGAAA |
| RAV1AAT | 316 (+) | CAACA |
| RAV1AAT | 235 (−) | CAACA |
| REALPHALGLHCB21 | 37 (+) | AACCAA |
| ROOTMOTIFTAPOX1 | 12 (+) | ATATT |
| TAAAGSTKST1 | 266 (+) | TAAAG |
| TATABOX3 | 124 (+) | TATTAAT |
| WRKY71OS | 9 (−) | TGAC |

TABLE 7
PLACE analysis results of the ZmGSStuc11-12-04.64626.1 upstream region

| SITE_NAME | POSITION (STRAND) | CONSENSUS |
|---|---|---|
| -10PEHVPSBD | 586 (+) | TATTCT |
| -10PEHVPSBD | 690 (+) | TATTCT |
| -10PEHVPSBD | 914 (−) | TATTCT |
| -300CORE | 19 (−) | TGTAAAG |
| -300ELEMENT | 232 (+) | TGHAAARK |
| -300ELEMENT | 820 (+) | TGHAAARK |
| -300ELEMENT | 18 (−) | TGHAAARK |
| AACACOREOSGLUB1 | 31 (+) | AACAAAC |
| AACACOREOSGLUB1 | 35 (+) | AACAAAC |
| AACACOREOSGLUB1 | 618 (−) | AACAAAC |
| AGMOTIFNTMYB2 | 132 (+) | AGATCCAA |
| AMYBOX1 | 385 (+) | TAACARA |
| AMYBOX1 | 619 (−) | TAACARA |
| AMYBOX2 | 217 (+) | TATCCAT |
| AMYBOX2 | 517 (−) | TATCCAT |
| ANAERO1CONSENSUS | 30 (+) | AAACAAA |
| ANAERO1CONSENSUS | 34 (+) | AAACAAA |
| ARR1AT | (+) | NGATT |
| ARR1AT | 396 (+) | NGATT |
| ARR1AT | 404 (−) | NGATT |
| ARR1AT | 897 (−) | NGATT |
| ARR1AT | 941 (−) | NGATT |
| ARR1AT | 1018 (−) | NGATT |
| BIHD1OS | 184 (+) | TGTCA |
| BIHD1OS | 325 (+) | TGTCA |
| BIHD1OS | 86 (−) | TGTCA |
| BOXIINTPATPB | 166 (+) | ATAGAA |
| BOXIINTPATPB | 530 (+) | ATAGAA |
| BOXIINTPATPB | 588 (−) | ATAGAA |
| CAATBOX1 | 226 (+) | CAAT |
| CAATBOX1 | 633 (+) | CAAT |
| CAATBOX1 | 715 (+) | CAAT |
| CAATBOX1 | 944 (+) | CAAT |
| CAATBOX1 | 222 (−) | CAAT |
| CAATBOX1 | (−) | CAAT |
| CAATBOX1 | 826 (−) | CAAT |
| CACTFTPPCA1 | 830 (+) | YACT |
| CACTFTPPCA1 | 380 (+) | YACT |
| CACTFTPPCA1 | 512 (+) | YACT |
| CACTFTPPCA1 | 784 (+) | YACT |
| CACTFTPPCA1 | 870 (+) | YACT |
| CACTFTPPCA1 | 44 (−) | YACT |
| CACTFTPPCA1 | 182 (−) | YACT |
| CACTFTPPCA1 | 193 (−) | YACT |
| CACTFTPPCA1 | 230 (−) | YACT |
| CACTFTPPCA1 | 237 (−) | YACT |
| CACTFTPPCA1 | 260 (−) | YACT |
| CACTFTPPCA1 | 344 (−) | YACT |
| CACTFTPPCA1 | 606 (−) | YACT |
| CACTFTPPCA1 | 818 (−) | YACT |
| CACTFTPPCA1 | 864 (−) | YACT |
| CACTFTPPCA1 | 978 (−) | YACT |
| CACTFTPPCA1 | 997 (−) | YACT |
| CARGATCONSENSUS | 714 (+) | CCWWWWWWGG |
| CARGATCONSENSUS | 714 (−) | CCWWWWWWGG |
| CARGCW8GAT | 328 (+) | CWWWWWWWWG |
| CARGCW8GAT | 388 (+) | CWWWWWWWWG |
| CARGCW8GAT | 328 (−) | CWWWWWWWWG |
| CARGCW8GAT | 388 (−) | CWWWWWWWWG |
| CATATGGMSAUR | 991 (+) | CATATG |
| CATATGGMSAUR | 991 (−) | CATATG |
| CBFHV | (+) | RYCGAC |
| CCAATBOX1 | 632 (+) | CCAAT |
| CCAATBOX1 | 714 (+) | CCAAT |
| CEREGLUBOX1PSLEGA | 381 (−) | TGTTAA |
| CPBCSPOR | 575 (+) | TATTAG |
| CPBCSPOR | (−) | TATTAG |
| CURECORECR | 723 (+) | GTAC |
| CURECORECR | 869 (+) | GTAC |
| CURECORECR | 723 (−) | GTAC |
| CURECORECR | 869 (−) | GTAC |
| DOFCOREZM | 170 (+) | AAAG |
| DOFCOREZM | (+) | AAAG |
| DOFCOREZM | 191 (+) | AAAG |
| DOFCOREZM | 235 (+) | AAAG |
| DOFCOREZM | 365 (+) | AAAG |
| DOFCOREZM | 414 (+) | AAAG |
| DOFCOREZM | 455 (+) | AAAG |
| DOFCOREZM | 468 (+) | AAAG |
| DOFCOREZM | 525 (+) | AAAG |
| DOFCOREZM | 604 (+) | AAAG |
| DOFCOREZM | 628 (+) | AAAG |
| DOFCOREZM | 850 (+) | AAAG |
| DOFCOREZM | 976 (+) | AAAG |
| DOFCOREZM | 19 (−) | AAAG |
| DOFCOREZM | (−) | AAAG |
| DOFCOREZM | 253 (−) | AAAG |
| DOFCOREZM | 382 (−) | AAAG |
| DOFCOREZM | 674 (−) | AAAG |
| DOFCOREZM | 699 (−) | AAAG |
| DOFCOREZM | 736 (−) | AAAG |
| DOFCOREZM | 798 (−) | AAAG |
| DOFCOREZM | 1001 (−) | AAAG |
| DRE2COREZMRAB17 | (+) | ACCGAC |
| DRECRTCOREAT | (+) | RCCGAC |
| E2FCONSENSUS | 72 (+) | WTTSSCSS |
| EBOXBNNAPA | 137 (+) | CANNTG |
| EBOXBNNAPA | 665 (+) | CANNTG |
| EBOXBNNAPA | 991 (+) | CANNTG |
| EBOXBNNAPA | 137 (−) | CANNTG |
| EBOXBNNAPA | 665 (−) | CANNTG |
| EBOXBNNAPA | 991 (−) | CANNTG |
| EECCRCAH1 | 889 (+) | GANTTNC |
| GAREAT | 59 (+) | TAACAAR |
| GAREAT | 385 (+) | TAACAAR |
| GAREAT | 619 (−) | TAACAAR |
| GATABOX | 92 (+) | GATA |
| GATABOX | 142 (+) | GATA |
| GATABOX | 173 (+) | GATA |
| GATABOX | (+) | GATA |
| GATABOX | 427 (+) | GATA |
| GATABOX | 449 (+) | GATA |
| GATABOX | 520 (+) | GATA |
| GATABOX | 529 (+) | GATA |
| GATABOX | 544 (+) | GATA |
| GATABOX | 815 (+) | GATA |
| GATABOX | 1006 (+) | GATA |
| GATABOX | 156 (−) | GATA |
| GATABOX | (−) | GATA |
| GATABOX | (−) | GATA |
| GATABOX | 217 (−) | GATA |
| GATABOX | 291 (−) | GATA |
| GATABOX | 349 (−) | GATA |
| GATABOX | 546 (−) | GATA |
| GATABOX | 833 (−) | GATA |
| GATABOX | 957 (−) | GATA |
| GATABOX | 1008 (−) | GATA |
| GATABOX | 1022 (−) | GATA |
| GT1CONSENSUS | 8 (+) | GRWAAW |
| GT1CONSENSUS | 173 (+) | GRWAAW |
| GT1CONSENSUS | 420 (+) | GRWAAW |
| GT1CONSENSUS | 421 (+) | GRWAAW |
| GT1CONSENSUS | 427 (+) | GRWAAW |
| GT1CONSENSUS | 592 (−) | GRWAAW |
| GT1CONSENSUS | 154 (−) | GRWAAW |
| GT1CONSENSUS | 289 (−) | GRWAAW |
| GT1CONSENSUS | 955 (−) | GRWAAW |
| GT1GMSCAM4 | 8 (+) | GAAAAA |
| GTGANTG10 | 607 (+) | GTGA |
| GTGANTG10 | 938 (+) | GTGA |
| GTGANTG10 | 1015 (+) | GTGA |
| GTGANTG10 | (−) | GTGA |
| GTGANTG10 | 661 (−) | GTGA |
| IBOX | 142 (+) | GATAAG |
| IBOXCORE | 142 (+) | GATAA |
| IBOXCORE | 173 (+) | GATAA |
| IBOXCORE | 427 (+) | GATAA |
| IBOXCORE | 155 (−) | GATAA |
| IBOXCORE | 290 (−) | GATAA |
| IBOXCORE | 956 (−) | GATAA |
| IBOXCORENT | 142 (+) | GATAAGR |
| INRNTPSADB | 950 (+) | YTCANTYY |
| LEAFYATAG | 632 (+) | CCAATGT |
| LECPLEACS2 | 569 (−) | TAAAATAT |
| LTRECOREATCOR15 | 433 (+) | CCGAC |
| MYB1AT | 711 (+) | WAACCA |
| MYB1AT | 987 (+) | WAACCA |
| MYB1LEPR | 556 (−) | GTTAGTT |
| MYB1LEPR | 921 (−) | GTTAGTT |
| MYB2CONSENSUSAT | 665 (−) | YAACKG |
| MYBATRD22 | 710 (+) | CTAACCA |
| MYBCORE | 665 (+) | CNGTTR |
| MYBCOREATCYCB1 | 855 (+) | AACGG |
| MYBCOREATCYCB1 | 792 (−) | AACGG |
| MYBGAHV | 385 (+) | TAACAAA |
| MYBGAHV | 619 (−) | TAACAAA |
| MYBPZM | 908 (−) | CCWACC |
| MYBST1 | 448 (+) | GGATA |
| MYBST1 | 519 (+) | GGATA |
| MYBST1 | 528 (+) | GGATA |
| MYBST1 | 814 (+) | GGATA |
| MYBST1 | 1005 (+) | GGATA |
| MYBST1 | 156 (−) | GGATA |
| MYBST1 | 217 (−) | GGATA |
| MYBST1 | 833 (−) | GGATA |
| MYCCONSENSUSAT | 137 (+) | CANNTG |
| MYCCONSENSUSAT | 665 (+) | CANNTG |
| MYCCONSENSUSAT | 991 (+) | CANNTG |
| MYCCONSENSUSAT | 137 (−) | CANNTG |
| MYCCONSENSUSAT | 665 (−) | CANNTG |
| MYCCONSENSUSAT | 991 (−) | CANNTG |
| NODCON1GM | 170 (+) | AAAGAT |
| NODCON1GM | (+) | AAAGAT |
| NODCON1GM | 414 (+) | AAAGAT |
| NODCON1GM | 468 (+) | AAAGAT |
| NTBBF1ARROLB | 381 (+) | ACTTTA |
| OSE1ROOTNODULE | 170 (+) | AAAGAT |
| OSE1ROOTNODULE | (+) | AAAGAT |
| OSE1ROOTNODULE | 414 (+) | AAAGAT |
| OSE1ROOTNODULE | 468 (+) | AAAGAT |
| POLASIG1 | 390 (+) | AATAAA |
| POLASIG1 | 845 (+) | AATAAA |
| POLASIG1 | 1040 (+) | AATAAA |
| POLASIG1 | 573 (−) | AATAAA |
| POLASIG1 | 801 (−) | AATAAA |
| POLASIG2 | 738 (−) | AATTAAA |
| POLASIG3 | 961 (−) | AATAAT |
| POLLEN1LELAT52 | 7 (+) | AGAAA |
| POLLEN1LELAT52 | 168 (+) | AGAAA |
| POLLEN1LELAT52 | 363 (+) | AGAAA |
| POLLEN1LELAT52 | 532 (+) | AGAAA |
| POLLEN1LELAT52 | 852 (+) | AGAAA |
| POLLEN1LELAT52 | 877 (+) | AGAAA |
| POLLEN1LELAT52 | 982 (+) | AGAAA |
| POLLEN1LELAT52 | 7 (+) | AGAAA |
| POLLEN1LELAT52 | 594 (−) | AGAAA |
| POLLEN1LELAT52 | 772 (−) | AGAAA |
| POLLEN1LELAT52 | 795 (−) | AGAAA |
| PREATPRODH | 994 (−) | ACTCAT |
| PRECONSCRHSP70A | 433 (+) | SCGAYNRNNNNNNN |
| | | NNNNNNNNHD |
| PRECONSCRHSP70A | 244 (+) | SCGAYNRNNNNNNN |
| | | NNNNNNNNHD |
| PROLAMINBOXOSGLUB1 | 232 (+) | TGCAAAG |
| PROLAMINBOXOSGLUB1 | (+) | TGCAAAG |
| PYRIMIDINEBOXOSRAMY1A | 673 (+) | CCTTTT |
| PYRIMIDINEBOXOSRAMY1A | 735 (+) | CCTTTT |
| PYRIMIDINEBOXOSRAMY1A | 454 (−) | CCTTTT |
| RAV1AAT | 538 (+) | CAACA |
| RAV1AAT | 552 (+) | CAACA |
| RAV1AAT | 600 (+) | CAACA |
| RAV1AAT | 726 (+) | CAACA |
| RAV1AAT | 885 (−) | CAACA |
| RAV1AAT | 932 (−) | CAACA |
| REALPHALGLHCB21 | 712 (+) | AACCAA |
| ROOTMOTIFTAPOX1 | 376 (+) | ATATT |
| ROOTMOTIFTAPOX1 | 569 (+) | ATATT |
| ROOTMOTIFTAPOX1 | 585 (+) | ATATT |
| ROOTMOTIFTAPOX1 | 347 (−) | ATATT |
| ROOTMOTIFTAPOX1 | 375 (−) | ATATT |
| SEF4MOTIFGM7S | (−) | RTTTTTR |
| SREATMSD | 155 (+) | TTATCC |
| SV40COREENHAN | 305 (−) | GTGGWWHG |
| TAAAGSTKST1 | (+) | TAAAG |
| TAAAGSTKST1 | 19 (−) | TAAAG |
| TAAAGSTKST1 | (−) | TAAAG |
| TAAAGSTKST1 | 382 (−) | TAAAG |
| TATABOX2 | 491 (−) | TATAAAT |
| TATABOX3 | 125 (+) | TATTAAT |
| TATABOX3 | 126 (−) | TATTAAT |
| TATABOX4 | 779 (−) | TATATAA |
| TATABOX5 | 369 (+) | TTATTT |
| TATABOX5 | 802 (+) | TTATTT |
| TATABOX5 | 56 (−) | TTATTT |
| TATABOX5 | 389 (−) | TTATTT |
| TATABOX5 | 844 (−) | TTATTT |
| TATABOX5 | 984 (−) | TTATTT |
| TATABOX5 | 1 (−) | TTATTT |
| TATABOXOSPAL | 370 (+) | TATTTAA |
| TATCCAOSAMY | 217 (+) | TATCCA |
| TATCCAOSAMY | 833 (+) | TATCCA |
| TATCCAOSAMY | 518 (−) | TATCCA |
| TATCCAOSAMY | 813 (−) | TATCCA |
| TATCCAOSAMY | 1004 (−) | TATCCA |
| TATCCAYMOTIFOSRAMY3D | 217 (+) | TATCCAY |
| TATCCAYMOTIFOSRAMY3D | 517 (−) | TATCCAY |
| TBOXATGAPB | 234 (−) | ACTTTG |
| TBOXATGAPB | 603 (−) | ACTTTG |
| UP2ATMSD | 283 (−) | AAACCCTA |
| WBOXNTERF3 | 501 (+) | TGACY |
| WBOXNTERF3 | 608 (+) | TGACY |
| WRKY71OS | 86 (+) | TGAC |
| WRKY71OS | 501 (+) | TGAC |
| WRKY71OS | 608 (+) | TGAC |
| WRKY71OS | 185 (−) | TGAC |
| WRKY71OS | 326 (−) | TGAC |

TABLE 8
PLACE analysis results of the ZmGSStuc11-12-04.16895.1 upstream region

| SITE_NAME | POSITION (STRAND) | CONSENSUS |
|---|---|---|
| -10PEHVPSBD | 698 (−) | TATTCT |
| 2SSEEDPROTBANAPA | 722 (+) | CAAACAC |
| 2SSEEDPROTBANAPA | 317 (−) | CAAACAC |
| 2SSEEDPROTBANAPA | 488 (−) | CAAACAC |
| ABRELATERD1 | 531 (−) | ACGTG |
| ACGTABOX | 327 (+) | TACGTA |
| ACGTABOX | 327 (−) | TACGTA |
| ACGTATERD1 | 328 (+) | ACGT |
| ACGTATERD1 | 532 (+) | ACGT |
| ACGTATERD1 | 328 (−) | ACGT |
| ACGTATERD1 | 532 (−) | ACGT |
| AMYBOX1 | 661 (+) | TAACARA |
| ANAERO2CONSENSUS | 19 (+) | AGCAGC |
| ANAERO2CONSENSUS | 22 (+) | AGCAGC |
| ANAERO2CONSENSUS | 69 (+) | AGCAGC |
| ANAERO2CONSENSUS | 72 (+) | AGCAGC |
| ANAERO2CONSENSUS | 737 (+) | AGCAGC |
| ANAERO2CONSENSUS | 200 (−) | AGCAGC |
| ARFAT | 460 (+) | TGTCTC |
| ARR1AT | 417 (+) | NGATT |
| ARR1AT | 291 (+) | NGATT |
| ARR1AT | 555 (−) | NGATT |
| ARR1AT | 703 (−) | NGATT |
| ASF1MOTIFCAMV | 440 (+) | TGACG |
| BOXLCOREDCPAL | 360 (+) | ACCWWCC |
| CAATBOX1 | 341 (+) | CAAT |
| CAATBOX1 | 108 (−) | CAAT |
| CAATBOX1 | 426 (−) | CAAT |
| CAATBOX1 | 438 (−) | CAAT |
| CACTFTPPCA1 | 219 (+) | YACT |
| CACTFTPPCA1 | 331 (+) | YACT |
| CACTFTPPCA1 | 43 (−) | YACT |
| ACTFTPPCA1 | 316 (−) | YACT |
| CACTFTPPCA1 | 346 (−) | YACT |
| CANBNNAPA | 722 (+) | CNAACAC |
| CANBNNAPA | 317 (−) | CNAACAC |
| CANBNNAPA | 488 (−) | CNAACAC |
| CAREOSREP1 | 392 (+) | CAACTC |
| CATATGGMSAUR | 380 (+) | CATATG |
| CATATGGMSAUR | 380 (−) | CATATG |
| CBFHV | 144 (+) | RYCGAC |
| CBFHV | 207 (+) | RYCGAC |
| CBFHV | 114 (−) | RYCGAC |
| CGACGOSAMY3 | 95 (+) | CGACG |
| CGACGOSAMY3 | 122 (+) | CGACG |
| CGACGOSAMY3 | 125 (+) | CGACG |
| CGACGOSAMY3 | 146 (+) | CGACG |
| CGACGOSAMY3 | 628 (+) | CGACG |
| CGACGOSAMY3 | 113 (−) | CGACG |
| CGACGOSAMY3 | 572 (−) | CGACG |
| CGCGBOXAT | 223 (+) | VCGCGB |
| CGCGBOXAT | 4 (+) | VCGCGB |
| CGCGBOXAT | 223 (−) | VCGCGB |
| CGCGBOXAT | 4 (−) | VCGCGB |
| CIACADIANLELHC | 366 (+) | CAANNNNATC |
| CURECORECR | 44 (+) | GTAC |
| CURECORECR | 330 (+) | GTAC |
| CURECORECR | 429 (+) | GTAC |
| CURECORECR | 44 (−) | GTAC |
| CURECORECR | 330 (−) | GTAC |
| CURECORECR | 429 (−) | GTAC |
| DOFCOREZM | 667 (+) | AAAG |
| DOFCOREZM | 671 (+) | AAAG |
| DOFCOREZM | 676 (+) | AAAG |
| DOFCOREZM | 684 (+) | AAAG |
| DOFCOREZM | 312 (−) | AAAG |
| DPBFCOREDCDC3 | 596 (+) | ACACNNG |
| DPBFCOREDCDC3 | 725 (+) | ACACNNG |
| DPBFCOREDCDC3 | 454 (−) | ACACNNG |
| DRE1COREZMRAB17 | 705 (−) | ACCGAGA |
| DRECRTCOREAT | 207 (+) | RCCGAC |
| DRECRTCOREAT | 114 (−) | RCCGAC |
| EBOXBNNAPA | 24 (+) | CANNTG |
| EBOXBNNAPA | 380 (+) | CANNTG |
| EBOXBNNAPA | 446 (+) | CANNTG |
| EBOXBNNAPA | 543 (+) | CANNTG |
| EBOXBNNAPA | 739 (+) | CANNTG |
| EBOXBNNAPA | 24 (−) | CANNTG |
| EBOXBNNAPA | 380 (−) | CANNTG |
| EBOXBNNAPA | 446 (−) | CANNTG |
| EBOXBNNAPA | 543 (−) | CANNTG |
| EBOXBNNAPA | 739 (−) | CANNTG |
| ELRECOREPCRP1 | 354 (−) | TTGACC |
| GAREAT | 661 (+) | TAACAAR |
| GATABOX | 434 (+) | GATA |
| GATABOX | 611 (−) | GATA |
| GCCCORE | 117 (−) | GCCGCC |
| GT1CONSENSUS | 434 (+) | GRWAAW |
| GT1CONSENSUS | 548 (+) | GRWAAW |
| GT1CONSENSUS | 470 (−) | GRWAAW |
| GT1CONSENSUS | 471 (−) | GRWAAW |
| GT1GMSCAM4 | 548 (+) | GAAAAA |
| GTGANTG10 | 681 (+) | GTGA |
| GTGANTG10 | 537 (−) | GTGA |
| GTGANTG10 | 557 (−) | GTGA |
| GTGANTG10 | 613 (−) | GTGA |
| GTGANTG10 | 625 (−) | GTGA |
| HEXAMERATH4 | 112 (+) | CCGTCG |
| HEXAMERATH4 | 95 (−) | CCGTCG |
| HEXAMERATH4 | 125 (−) | CCGTCG |
| HEXAMERATH4 | 628 (−) | CCGTCG |
| IBOXCORE | 434 (+) | GATAA |
| INRNTPSADB | 467 (+) | YTCANTYY |
| LTRE1HVBLT49 | 413 (−) | CCGAAA |
| LTRECOREATCOR15 | 208 (+) | CCGAC |
| LTRECOREATCOR15 | 114 (−) | CCGAC |
| MYB2CONSENSUSAT | 31 (−) | YAACKG |
| MYBCORE | 31 (+) | CNGTTR |
| MYBCORE | 98 (+) | CNGTTR |
| MYBCORE | 366 (−) | CNGTTR |
| MYBCOREATCYCB1 | 634 (+) | AACGG |
| MYBCOREATCYCB1 | 31 (−) | AACGG |
| MYBCOREATCYCB1 | 617 (−) | AACGG |
| MYBGAHV | 661 (+) | TAACAAA |
| MYBPZM | 365 (+) | CCWACC |
| MYBST1 | 433 (+) | GGATA |
| MYCCONSENSUSAT | 24 (+) | CANNTG |
| MYCCONSENSUSAT | 380 (+) | CANNTG |
| MYCCONSENSUSAT | 446 (+) | CANNTG |
| MYCCONSENSUSAT | 543 (+) | CANNTG |
| MYCCONSENSUSAT | 739 (+) | CANNTG |
| MYCCONSENSUSAT | 24 (−) | CANNTG |
| MYCCONSENSUSAT | 380 (−) | CANNTG |
| MYCCONSENSUSAT | 446 (−) | CANNTG |
| MYCCONSENSUSAT | 543 (−) | CANNTG |
| MYCCONSENSUSAT | 739 (−) | CANNTG |
| POLASIG3 | 700 (+) | AATAAT |
| POLLEN1LELAT52 | 669 (+) | AGAAA |
| PRECONSCRHSP70A | 121 (+) | SCGAYNRNNNNNNNNNNNNNNNHD |
| PRECONSCRHSP70A | 95 (−) | SCGAYNRNNNNNNNNNNNNNNNHD |
| RAV1AAT | 35 (−) | CAACA |
| RAV1AAT | 495 (−) | CAACA |
| RAV1BAT | 543 (+) | CACCTG |
| REBETALGLHCB21 | 432 (+) | CGGATA |
| RHERPATEXPA7 | 530 (+) | KCACGW |
| RHERPATEXPA7 | 732 (+) | KCACGW |
| RHERPATEXPA7 | 625 (+) | KCACGW |
| S1FBOXSORPS1L21 | 335 (−) | ATGGTA |
| SEF4MOTIFGM7S | 710 (+) | RTTTTTR |
| IIATCYTC | 260 (+) | TGGGCY |
| IIATCYTC | 265 (+) | TGGGCY |
| IIATCYTC | 275 (+) | TGGGCY |
| SORLIP1AT | 588 (+) | GCCAC |
| SORLIP1AT | 103 (−) | GCCAC |
| SORLIP2AT | 266 (+) | GGGCC |
| SORLIP2AT | 276 (+) | GGGCC |
| SORLIP2AT | 605 (−) | GGGCC |
| SORLIP4AT | 347 (+) | GTATGATGG |
| SREATMSD | 433 (−) | TTATCC |
| SURECOREATSULTR11 | 171 (+) | GAGAC |
| SURECOREATSULTR11 | 461 (−) | GAGAC |
| SURECOREATSULTR11 | 534 (−) | GAGAC |
| TAAAGSTKST1 | 312 (−) | TAAAG |
| WBBOXPCWRKY1 | 354 (−) | TTTGACY |
| WBOXATNPR1 | 439 (+) | TTGAC |
| WBOXATNPR1 | 355 (−) | TTGAC |
| WBOXHVISO1 | 653 (−) | TGACT |
| WBOXNTCHN48 | 653 (−) | CTGACY |
| WBOXNTERF3 | 653 (−) | TGACY |
| WBOXNTERF3 | 354 (−) | TGACY |
| WRKY71OS | 440 (+) | TGAC |
| WRKY71OS | 355 (−) | TGAC |
| WRKY71OS | 654 (−) | TGAC |

Notwithstanding the variations in lengths of the promoters and 5′ upstream regulatory sequences analysed, the data presented in Table 4 to Table 8 indicate the presence of several conserved structural features, including e.g., a plurality of each element in the group consisting of an ARR1AT element, an ACGTATERD1 element, a CAATBOX1 element, a CACFTPPCA1 element, a CURECORECR element, a DOFCOREZM element, an EBOXBNNAPA element, a GATABOX element, a GT1CONSENSUS element, a GTGANTG10 element, and a MYCCONSENSUSAT element in the proximal 750 bp upstream of the translation start site. For example, these elements may each be represented at least 2 or 3 or 4 or 5 or 6 times in a given sequence. Alternatively, or in addition, these elements may be represented as many as 7 or 8 or 9 or 10 or 11 or more times in a given sequence. This means that the sequences may be present on either DNA strand, the only requirement being that they are identified by PLACE analysis.

Of these elements, CACFTPPCA1 elements, DOFCOREZM elements and GT1CONSENSUS elements are consistently highly-abundant with 4 or more occurrence of each in each sequence analyzed. If the shorter rice sequence is excluded from the analysis, then the abundance of the ARR1AT elements, CURECORECR elements, DOFCOREZM elements, EBOXBNNAPA elements, GTGANTG10 elements and MYCCONSENSUSAT elements are also observed to be highly abundant for maize and wheat sequences, with 4 or more occurrence of each element in the proximal 750 bp upstream of the translation start site.

The sequences are also characterized by the presence of at least one element in the group consisting of an IBOXCORE element (1, 2 or 6 occurrences), a MYB2CONSENSUS element, (one occurrence in each sequence), a MYBCORE element (1-3 occurrences) and a WRKY71OS element (1 or 3 or 5 or 7 occurrences) in the proximal 750 bp upstream of the translation start site. Excluding the shorter rice sequence, at least one occurrence of the MYBST1 and MYBCOREATCYCB1 and PRECONSCRHSP70A elements is also found at low copy number (generally 1 or 2 or 3 occurrences) in maize and wheat sequences i.e., the proximal 750 bp upstream of the translation start site.

1-73. (canceled)

74. An isolated promoter, or an active fragment or derivative thereof, capable of conferring selective or specific expression to a gene to which it is operably connected in the endosperm of a developing plant seed, wherein said promoter in its native context confers endosperm-selective expression or preferential endosperm expression to a genomic gene comprising a nucleotide sequence selected from the group consisting of:
(i) the nucleotide sequence of SEQ ID NO: 1 or 2;
(ii) a nucleotide sequence encoding a polypeptide having at least about 50% sequence identity to a polypeptide encoded by the nucleotide sequence of SEQ ID NO: 1 or 2, wherein said polypeptide is expressed selectively in endosperm of developing seed;
(iii) a nucleotide sequence that hybridizes under at least moderate stringency conditions to the sequence of (i) or (ii), or a complementary sequence thereto, wherein said hybridising sequence is expressed selectively in endosperm of developing seed; and
(iv) a nucleotide sequence having homology to the sequence of (i) or (ii) as determined by homology searching using the BLASTN algorithm with a nucleotide mismatch penalty (−q) of at least −1, wherein said homologous sequence is expressed selectively in endosperm of developing seed.

75. The isolated promoter, active fragment, or derivative of claim 74 comprising a nucleotide sequence selected from the group consisting of:
(i) the nucleotide sequence of SEQ ID NO: 3, 4, 5, 6, 7 or 8;
(ii) a nucleotide sequence complementary to the nucleotide sequence of (i);
(iii) a nucleotide sequence having at least about 70% sequence identity to the sequence of (i) or (ii); and
(iv) a nucleotide sequence amplified from genomic DNA using one or more amplification primers, wherein each of said primers comprises a sequence of at least about 12 contiguous nucleotides in length derived from the nucleotide sequence of SEQ ID NO: 1 or 2, or a complementary sequence thereto.

76. The isolated promoter, active fragment, or derivative of claim 74, wherein the promoter, fragment, or derivative is capable of conferring endosperm-selective, endosperm-specific, or preferential endosperm expression to a gene to which it is operably connected in developing seed of a monocotyledonous plant.

77. The isolated promoter, active fragment, or derivative of claim 74, wherein the promoter, fragment, or derivative is from a monocotyledonous plant.

78. The isolated promoter, active fragment, or derivative of claim 74, wherein the promoter, fragment, or derivative is capable of conferring endosperm-selective, endosperm-specific, or preferential endosperm expression to a gene to which it is operably connected during the period of from about 5 days after pollination (DAP) to at least about 25 DAP.

79. The isolated promoter, active fragment, or derivative of claim 74, wherein said promoter, fragment, or derivative does not confer detectable expression in vegetative tissues or organs, reproductive tissues or organs, floral tissues or organs, embryo, or endosperm of a mature seed.

80. The isolated promoter, active fragment, or derivative of claim 74, wherein said promoter, fragment, or derivative comprises one or more nucleotide sequences set forth in Table 4, 5, 6, 7 and/or 8.

81. The isolated promoter, active fragment, or derivative of claim 80, wherein said promoter, fragment, or derivative comprises one or more nucleotide sequences set forth in Table 1.

82. The isolated promoter, active fragment, or derivative of claim 80, wherein said promoter, fragment, or derivative comprises a plurality of each element in the group consisting of an ARR1AT element, an ACGTATERD1 element, a CAATBOX1 element, a CACFTPPCA1 element, a CURECORECR element, a DOFCOREZM element, an EBOXBNNAPA element, a GATABOX element, a GT1CONSENSUS element, a GTGANTG10 element, and a MYCCONSENSUSAT element, in the proximal 750 base pairs upstream of the translation start site of the corresponding genomic gene from which it is derived.

83. The isolated promoter, active fragment, or derivative of claim 82, wherein each element is represented at least 2, 3, 4, 5 or 6 times in the proximal 750 base pairs upstream of the translation start site of the corresponding genomic gene from which it is derived.

84. The isolated promoter, active fragment, or derivative of claim 82, wherein the CACFTPPCA1 elements, DOFCOREZM elements, and GT1CONSENSUS elements are each represented at least 4 times in the proximal 750 base pairs upstream of the translation start site of the corresponding genomic gene from which it is derived.

85. The isolated promoter, active fragment, or derivative of claim 82, wherein the ARR1AT elements, CURECORECR elements, DOFCOREZM elements, EBOXBNNAPA elements, GTGANTG10 elements, and MYCCONSENSUSAT elements are each represented at least 4 times in the proximal 750 base pairs upstream of the translation start site of the corresponding genomic gene from which it is derived.

86. The isolated promoter, active fragment, or derivative of claim 82, further comprising at least one element selected from the group consisting of an IBOXCORE element, a MYB2CONSENSUS element, a MYBCORE element, and a WRKY710S element in the proximal 750 base pairs upstream of the translation start site.

87. The isolated promoter, active fragment, or derivative of claim 82, further comprising at least one element selected from the group consisting of a MYBST1 element, a MYBCOREATCYCB1 element, and a PRECONSCRHSP70A element in the proximal 750 base pairs upstream of the translation start site.

88. An expression construct comprising the isolated promoter of claim 74, or an active fragment or derivative thereof, operably connected to a transgene.

89. An expression vector comprising
(i) the isolated promoter of claim 74, or an active fragment or derivative thereof; or
(ii) an expression construct comprising the promoter, fragment, or derivative of (i) operably connected to a transgene.

90. A method for producing an expression construct, comprising linking the isolated promoter of claim 74, or an active fragment or derivative thereof, to a transgene such that the promoter, fragment, or derivative is capable of conferring endosperm-selective, endosperm-specific, or preferential endosperm expression to said transgene in a cell.

91. A process for producing an expression vector, comprising linking the isolated promoter of claim 74, or an active fragment or derivative thereof, to an empty vector to thereby produce an expression vector.

92. A process for producing an expression vector, comprising linking the expression construct of claim 88 to an empty vector to thereby produce an expression vector.

93. A transgenic plant, plant part, or plant cell comprising
(i) the isolated promoter of claim 74, or an active fragment or derivative thereof;
(ii) an expression construct comprising the promoter, fragment or derivative of (i) operably connected to a transgene; or
(iii) an expression vector comprising the promoter, fragment or derivative of (i) or the expression construct of (ii).

94. The transgenic plant, plant part, or plant cell of claim 93, wherein the expression construct is integrated into the genome of the plant, plant part, or plant cell.

95. A method for producing a transgenic plant, plant part, or plant cell, said method comprising introducing into the plant, plant part, or plant cell
(i) the isolated promoter of claim 74, or an active fragment or derivative thereof;
(ii) an expression construct comprising the promoter, fragment, or derivative of (i) operably connected to a transgene; or
(iii) an expression vector comprising the promoter, fragment, or derivative of (i) or the expression construct of (ii).

96. A method for producing a transgenic plant, plantlet, or plant part, said method comprising:
(i) obtaining the transgenic plant cell produced by the method of claim 95; and
(ii) regenerating the transgenic plant cell of (i), to produce a transgenic plant, plantlet, or plant part.

97. A method of modulating expression of a transgene in developing endosperm comprising transforming a plant with
(i) a transgene operably connected to the promoter of claim 74, or an active fragment or derivative of said promoter;
(ii) an expression construct comprising a transgene and the promoter of claim 74, or an active fragment or derivative of said promoter; or
(iii) an expression vector comprising the expression construct of (ii).


SEQUENCE LISTING (19 sequences)

SEQ ID NO: 1 - DNA, 1409 bp, Triticum aestivum
gcacgagccgaggccagcgctgccggcgccgcagcagcgggcggtggacgggttctggagggagcggcaggaggagatggaggcgacggcggacttcaacgaccgcatactgcccatggcccgcctcaagaggctcatccgcgccgaggaggacggcatgatgatcgccgccgacacgccggcgtacctggccaagctctgcgagctcttcgtgcaggagctcgccgtgcgcgcctgggcgtgcgcccaatcccaccaccgccgcatcatactggaatcggacatcgccgaggccatcgccttcacccagtcgtacgacttcctcgccaccgtgctcctcgagcaccaacgggaggcgcggctggccggccgtgctgctatcccgacaacggttccggtgacggcggcgagggcaaggctcatcaccaggaagcgccacatgccggacccgaatcctccacggccggtgcatggggtgcggagaattcgtcctcgtgcgcttcctatcccgccgccgtcggactttcgctacgtgccggttccatttccgttcacctcggcgccgataggagccgcagcgatggcggaggggctgatgattctcccacccatcaaccacgcgactaccgagcgcgtgttcttcctggacaggaacagcggcactgacttcgcaggtgaaaactctgctgctgaaactatagcatctccgcctcctccggcagggcctgcaggagcagtggcgctgcccactgtccatcctgctgcttactacttgtgcgcttacccggtgaccaacgacgttgaggcctttgccgttggcaacactgatcctgatgtcatcccaccggagattgtagtgggagacgtcgccatcccaccggagattatagagggaaacgtcgccgatggcaacggcgacggcggacagcagcagcagcagagcgaaaaccttggtggtaatggtgagagtgtggtggtgtcgcaaagcaatggtgtgcaggaagatggtgcagatgggatgtttctgaaggagatcctcatggatgaagacctgatgtttcccgacgctgagctttttccgttggtgggcgctgcacctggtccagaggatttcatcgtcgaccaagatgttctcgacgacgtcttcgccaacccgagcagcagcgcaagcagcgactgaaccgaaagaagatcagagcgggacgcagcatcggttgattcatctatcgtctctcgacctgctactctatgctagccgctatatcggttaataaatttgggaataagtttgtgttcgtgcgtgtgacatggactgtatggttcgccctgaatttatcgtattgcaatatatagccgtgattgtgtgtaaaaaaaaaaaaaaaaaaaaaaaaaaaaaaaaaaaaaaaaaa

SEQ ID NO: 2 - DNA, 798 bp, Triticum aestivum
ggggatgtggcaacgggcagaggcagcgagaaggtgcacgtggcaatctactacgagtcgctgtgcccctactcggtccgcttcgtggcgaaccacctcttcaaggcctacagggacggcctgctcgacgccgccaatctcaccctcgtcccctatggcaacgccgtggtccgcaatgacggcaccatctcctgtcagcatggccccgaggaatgccttctcaacaccgtggaggcttgcgccatcgacgcctggccggacgtgaaggtgcatctcggcttcatttactgcgtgtcggacctggtgttgaagaacaagcaccgggagtgggagtcgtgcttccagaaacaggggcttgacccaaagccagtcacggagtgctacaagggcgagcgtggccacaatctatcgctcgagtatgggagacagacagctgagctcgtgcctcctcaccagttcgttccatgggtggtggtcgacggcaagccgctctacaacgattacgggaacttcaaagcttacatctgcaaggcgtacaagggctatcccctcctcgaggcgtgtcgaagcctgggcctggaggcacacgacgatgtgtacggccgactttgatcgtgatgatgatgatgccatcgccaagattaggatggtcctgccggatggtgataataatgattgataaatttatgtttcctctcggggatcatacatctgaacatagggcagttcaatctctctgttaaggttaataaatcaagtggttgtgctctaaaaaaaaaaaaaaaaaaaaaaaaaaaa

SEQ ID NO: 3 - DNA, 1279 bp, Triticum aestivum
tagctaaagttaaataaaaggctgaatttattgcatgggagaagagagagattgggtctggcacattgctcttagttctgcgtgtaaaatccagtgatggggtagctcctgaaataccaatatcttataagtaaagaaaagttttttatacaatacatatgtcaatatacggtgcaaggtgtcgctagctgactcgctaggcaggtgccggtaggtatcggcaccgctcaatttttcactttaatacatactagcatcccccggattgtagcattttaccacaaaaatactagcaaagggcattactcactttttagctaacttgtgaacattcactatatagaaaattccaaatgataggtttgtcactcttgagagggcgccaaagttggatttgtctccacatataaagggctaaacagaaagccagctggtgcttatgagatagaaactgatcacagaacgcatggatcaaaccccttgcatgcgtaccttcccaaaatagtggctagcaaaccacggacacatacatccatccttgagtttatcttaggatgaaacatccatccttgagcatcacggaaaaatacacaaacagaaagcgcgagtggacggctgtatgcagcggaaagcatccaaaatgggaagctgtatcctgtatgtatgtatgcagccatctcttcccggcgtcgtttctggcccagacggctcacacacacgcccctcgcaccctgaaggctgctgtgcagtacacgttgagaggcgctctacggtccatagataagcatgcatgcgtgcatgcatggccggagaagcaagggaagaacgctgcagagacggtgagcaccaggaccaggtgtaggtgcaaccagtcttgcgtgaattagggtggcacgcatccagacctttttaagattttaattaacacgagggccgtttgtgtgcttggctcaagcgcgcgtggggacaaaagctgtttcagcaaagcaatggacagatacgtgctgctaaggctgaagcaacacgggagttcatactggcatggcatcccctgggtcctatataaaggccaccctcctcgtgtccaccttcccactcatcgcaacccacctctggcttccaagtctatacataatatagggaccgagcttgcggttttgccaaggtaacacatctagtatctgcttagcttggttcttcttacagatactcctaagattgagcattaactgatcgattcgatggtacgtgcgtgcatgcaaacacaaaacgcagggtg

SEQ ID NO: 4 - DNA, 2400 bp, Triticum aestivum
ttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgcatggttacgtatacactaaacgtaccgaatgtcagagcacgcagttgtgctgctgtctcgcactcttatagaacggcaaaatcacgctcaccttatcttggattgcttatgtacatatcctgggaaatacgtcagcttgtcaatccaaaactattttctagatggagctatgcgctttcagttagagagtaagtccatcgtgacactgcagcgaactcgttcacctctttccagcacccggctgcacacgcggccgcccggttggcgctaatcaccttctataacactttgccatcacctctgccgcattgctcgatcggctgagcgaacacca

SEQ ID NO: 5 - DNA, 2066 bp, Triticum aestivum
ttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgc

SEQ ID NO: 6 - DNA, 330 bp, Oryza sativa
atccttgtgtcatattgtgttcatcttcatactaccaaccaattagtgctaataggtagcattttctgcagttgcagctcaagtaaggtaatgcacctactagcttattattcttgtgcctagtattaattagtttctatactggtacttgtgaaaatttgtgatttgagcgatgctatttgatttgtaattaagctagctatctcttttttcttggaccgcaatgtaacatcgtgttgctaacttttaaactataaaagtaagataaagaagtaacaaaagtgtgtgcttgctatgtaggtctattattcggagcaacaaagggaagga

SEQ ID NO: 7 - DNA, 1044 bp, Zea mays
gtaactagaaaaacacacctttacaccacaaacaaacaaacatagtaatagagccaaataacaagttaatgtttccccccttaaatgacacgatacacaccttcacaccatagacaaacatccttattaatagatccaaatgataaggtaacctttatcctatgaatagaaagataaagatagtgtcataaaagtgtatatctatctctatacatgtatccattgcaatagtgcaaagtataagcgacaagcctttgagagtgtctaataggttgcacaaaatagggttttatcttgatccaagcattccacgagcatcttagttgtcatatatatgtccacaagtaatatctaggagaaccagaaagttatttaatattactttaacaaataaatgattaaaaatcagcaccaaagatggaaaatgataaaccgactatagcttgaggatataaaaggaatggtgcaaagattgcatcatacataaaacatttataaaatgaccaaaatttacttatggatacaaaggatagaaaacaacaagatatcatcaacaactaacaaccttatattttattagttttatattctattttctacaacaaagtgacccctaggtttgttataaaagccaatgtatgtggtgcacattctcatcgatcaccagttgtgccttttggagttttagctattctacgctttgagagttctaaccaattaaggtacaacatctaccttttaattcttgaaattagtttatatgcgggcatgtttctagttatatactccctccgtttctttttatttgtcgctggatagtgtaaaattgcactatccagcgacaaataaaaagaaacggagggagtatgtacttcgagaaattatgttgagttccaaatctatgcttggtaggagaatacaactaacaacttgttgggtgaatcaatagctcattttatcattattaaaatacaaaaagtaagaaataaccatatgagtgctttggatatctaggtgaatctatcaagaccaaacaagaaataa

SEQ ID NO: 8 - DNA, 751 bp, Zea mays
cggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctccttcttctcggatgggctgggccggcatgggcctgctgaacgctgattaattaactcaagactgctttagtgtttgctatacgtacttaccatcaattagtatgatggtcaaaccttccaaccggatccattcatatgcttgcacaactctgggagtctgggtgttttcggattacaaattgtacggataattgacgccatttgtgcgtgtgtgtctcattcattttcctagaggaaactgtgtttgtgttgtgtggttcaagctgccgctggtataacttggcacgtctcacggcacctgaaaaaaaatcacgggcaggcttgcgtcgttgcatcggtcgccaccacacaccggccggcccctatcacccgtttcctcacgacggaacgggacgatgccgcagcagtcagcgtaacaaaaagaaagcaaagggtgaaaggggaagggggagaataatctcggtttttagcacgcaaacacacggcacgagcagctgagcgccc

SEQ ID NO: 9 - DNA, 491 bp, Zea mays
gggctgggccggcatgggcctgctgaacgctgattaattaactcaagactgctttagtgtttgctatacgtacttaccatcaattagtatgatggtcaaaccttccaaccggatccattcatatgcttgcacaactctgggagtctgggtgttttcggattacaaattgtacggataattgacgccatttgtgcgtgtgtgtctcattcattttcctagaggaaactgtgtttgtgttgtgtggttcaagctgccgctggtataacttggcacgtctcacggcacctgaaaaaaaatcacgggcaggcttgcgtcgttgcatcggtcgccaccacacaccggccggcccctatcacccgtttcctcacgacggaacgggacgatgccgcagcagtcagcgtaacaaaaagaaagcaaagggtgaaaggggaagggggagaataatctcggtttttagcacgcaaacacacggcacgagcagctgagcgccc

SEQ ID NO: 10 - DNA, 7581 bp, Artificial Sequence
  Vector pBSubn R4R3 comprising Ubi::bar-nos selection cassette and the R4R3 multi-site Gateway entry point for promoter, reporter gene and termination sequence Entry Clones.
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcattttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgaattcctgcagccccgccaagctatcaactttgtatagaaaagttgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggggactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcggcaaaagagccgttcatttcaataaaccgggcgacctcagccatcccttcctgattttccgctttccagcgttcggcacgcagacgacgggcttcattctgcatggttgtgcttaccgaaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatcctctagattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctgggggtgcctaatgcggccgccatagtgactggatatgttgtgttttacagtattatgtagtctgttttttatgcaaaatctaatttaatatattgatatttatatcattttacgtttctcgttcaactttattatacatagttgataattcactggccgtcgtgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 11 - DNA, 10767 bp, Artificial Sequence
  Vector pPZP200 35S hph 35S R4R3 comprising 35S::hph-35S selection cassette and the R4R3 multi-site Gateway entry point for promoter, reporter gene and termination sequence Entry Clones.
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcgagctcggtacccggggatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattaggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatccttatagagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtcttcatgatgaagtgacagatagctgggcaatggaatccgaggaggtttcccgaaattaccctttgttggaaagtctcaattgccctttggtcttctgagactgtatccttgatatttttggagtagaccagagtgtcgtgctccaccatgttgacgaagattttcttcttgtcattgagtcgtaagagactctgtatgaactgttcgccagttttcacggcgagttctgttagatcctcgatttgaatctttgactccatggcctttgattcagtaggaactacttttttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttaatgagacctgctgcgtaggcctctctaaccatctgtgggttagcgttctttctgaaattgaagaggctaatcttctcattatcagtggtgaacatagtatcgtcaccttcaccgtcgaactttcttcctagatcgtagagatagaggaagtcgtccattgtaatctccggggcaaaggagatcctctagagtcgagggtacccggggatcctctagagtcgagggtacccggggatcctctagagtcgaatgattacgccaagctatcaactttgtatagaaaagttgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggggactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcggcaaaagagccgttcatttcaataaaccgggcgacctcagccatcccttcctgattttccgctttccagcgttcggcacgcagacgacgggcttcattctgcatggttgtgcttaccgaaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatcctctagattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctgggggtgcctaatgcggccgccatagtgactggatatgttgtgttttacagtattatgtagtctgttttttatgcaaaatctaatttaatatattgatatttatatcattttacgtttctcgttcaactttattatacatagttgataattcactggccgtcgttttacaactcgacctgcaggcatgcaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 12 - DNA, 11415 bp, Artificial Sequence
  Binary vector pMPB0098 comprising WP05::gfp-nos expression cassette and 35S::hph-35S selection cassette for use as Entry Clone with Gateway Destination Clone.
ttatacatagttgataattcactggccgtcgttttacaactcgacctgcaggcatgcaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcgagctcggtacccggggatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattaggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatccttatagagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtcttcatgatgaagtgacagatagctgggcaatggaatccgaggaggtttcccgaaattaccctttgttggaaagtctcaattgccctttggtcttctgagactgtatccttgatatttttggagtagaccagagtgtcgtgctccaccatgttgacgaagattttcttcttgtcattgagtcgtaagagactctgtatgaactgttcgccagttttcacggcgagttctgttagatcctcgatttgaatctttgactccatggcctttgattcagtaggaactacttttttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttaatgagacctgctgcgtaggcctctctaaccatctgtgggttagcgttctttctgaaattgaagaggctaatcttctcattatcagtggtgaacatagtatcgtcaccttcaccgtcgaactttcttcctagatcgtagagatagaggaagtcgtccattgtaatctccggggcaaaggagatcctctagagtcgagggtacccggggatcctctagagtcgagggtacccggggatcctctagagtcgaatgattacgccaagctatcaactttgtatagaaaagttgcttagctaaagttaaataaaaggctgaatttattgcatgggagaagagagagattgggtctggcacattgctcttagttctgcgtgtaaaatccagtgatggggtagctcctgaaataccaatatcttataagtaaagaaaagttttttatacaatacatatgtcaatatacggtgcaaggtgtcgctagctgactcgctaggcaggtgccggtaggtatcggcaccgctcaatttttcactttaatacatactagcatcccccggattgtagcattttaccacaaaaatactagcaaagggcattactcactttttagctaacttgtgaacattcactatatagaaaattccaaatgataggtttgtcactcttgagagggcgccaaagttggatttgtctccacatataaagggctaaacagaaagccagctggtgcttatgagatagaaactgatcacagaacgcatggatcaaaccccttgcatgcgtaccttcccaaaatagtggctagcaaaccacggacacatacatccatccttgagtttatcttaggatgaaacatccatccttgagcatcacggaaaaatacacaaacagaaagcgcgagtggacggctgtatgcagcggaaagcatccaaaatgggaagctgtatcctgtatgtatgtatgcagccatctcttcccggcgtcgtttctggcccagacggctcacacacacgcccctcgcaccctgaaggctgctgtgcagtacacgttgagaggcgctctacggtccatagataagcatgcatgcgtgcatgcatggccggagaagcaagggaagaacgctgcagagacggtgagcaccaggaccaggtgtaggtgcaaccagtcttgcgtgaattagggtggcacgcatccagacctttttaagattttaattaacacgagggccgtttgtgtgcttggctcaagcgcgcgtggggacaaaagctgtttcagcaaagcaatggacagatacgtgctgctaaggctgaagcaacacgggagttcatactggcatggcatcccctgggtcctatataaaggccaccctcctcgtgtccaccttcccactcatcgcaacccacctctggcttccaagtctatacataatatagggaccgagcttgcggttttgccaaggtaacacatctagtatctgcttagcttggttcttcttacagatactcctaagattgagcattaactgatcgattcgatggtacgtgcgtgcatgcaaacacaaaacgcagggtggcaagtttgtacaaaaaagcaggctttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccctcatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagatacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaacttta

SEQ ID NO: 13 - DNA, 8190 bp, Artificial Sequence
  Binary vector pMPB0099 comprising WP05::gfp-nos expression cassette and Ubi::bar-nos selection cassette for use as Entry Clone with Gateway Destination Clone.
ttatacatagttgataattcactggccgtcgtgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcattttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgaattcctgcagccccgccaagctatcaactttgtatagaaaagttgcttagctaaagttaaataaaaggctgaatttattgcatgggagaagagagagattgggtctggcacattgctcttagttctgcgtgtaaaatccagtgatggggtagctcctgaaataccaatatcttataagtaaagaaaagttttttatacaatacatatgtcaatatacggtgcaaggtgtcgctagctgactcgctaggcaggtgccggtaggtatcggcaccgctcaatttttcactttaatacatactagcatcccccggattgtagcattttaccacaaaaatactagcaaagggcattactcactttttagctaacttgtgaacattcactatatagaaaattccaaatgataggtttgtcactcttgagagggcgccaaagttggatttgtctccacatataaagggctaaacagaaagccagctggtgcttatgagatagaaactgatcacagaacgcatggatcaaaccccttgcatgcgtaccttcccaaaatagtggctagcaaaccacggacacatacatccatccttgagtttatcttaggatgaaacatccatccttgagcatcacggaaaaatacacaaacagaaagcgcgagtggacggctgtatgcagcggaaagcatccaaaatgggaagctgtatcctgtatgtatgtatgcagccatctcttcccggcgtcgtttctggcccagacggctcacacacacgcccctcgcaccctgaaggctgctgtgcagtacacgttgagaggcgctctacggtccatagataagcatgcatgcgtgcatgcatggccggagaagcaagggaagaacgctgcagagacggtgagcaccaggaccaggtgtaggtgcaaccagtcttgcgtgaattagggtggcacgcatccagacctttttaagattttaattaacacgagggccgtttgtgtgcttggctcaagcgcgcgtggggacaaaagctgtttcagcaaagcaatggacagatacgtgctgctaaggctgaagcaacacgggagttcatactggcatggcatcccctgggtcctatataaaggccaccctcctcgtgtccaccttcccactcatcgcaacccacctctggcttccaagtctatacataatatagggaccgagcttgcggttttgccaaggtaacacatctagtatctgcttagcttggttcttcttacagatactcctaagattgagcattaactgatcgattcgatggtacgtgcgtgcatgcaaacacaaaacgcagggtggcaagtttgtacaaaaaagcaggctttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccctcatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagatacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaacttta

SEQ ID NO: 14 - DNA, 12202 bp, Artificial Sequence
  Binary vector pMPB0084 comprising WP07::gfp-nos expression cassette wherein WP07 promoter is 2066bp in length, and 35S::hph-35S selection cassette for use as Entry Clone with Gateway Destination Clone.
ttatacatagttgataattcactggccgtcgttttacaactcgacctgcaggcatgcaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcgagctcggtacccggggatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattaggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatccttatagagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtcttcatgatgaagtgacagatagctgggcaatggaatccgaggaggtttcccgaaattaccctttgttggaaagtctcaattgccctttggtcttctgagactgtatccttgatatttttggagtagaccagagtgtcgtgctccaccatgttgacgaagattttcttcttgtcattgagtcgtaagagactctgtatgaactgttcgccagttttcacggcgagttctgttagatcctcgatttgaatctttgactccatggcctttgattcagtaggaactacttttttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttaatgagacctgctgcgtaggcctctctaaccatctgtgggttagcgttctttctgaaattgaagaggctaatcttctcattatcagtggtgaacatagtatcgtcaccttcaccgtcgaactttcttcctagatcgtagagatagaggaagtcgtccattgtaatctccggggcaaaggagatcctctagagtcgagggtacccggggatcctctagagtcgagggtacccggggatcctctagagtcgaatgattacgccaagctatcaactttgtatagaaaagttgctttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgcgcaagtttgtacaaaaaagcaggctttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccctcatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagatacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaacttta

SEQ ID NO: 15 - DNA, 8977 bp, Artificial Sequence
  Binary vector pMPB0085 comprising WP07::gfp-nos expression cassette wherein WP07 promoter is 2066bp in length, and ubi::bar-nos selection cassette for use as Entry Clone with Gateway Destination Clone.
ttatacatagttgataattcactggccgtcgtgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcattttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgaattcctgcagccccgccaagctatcaactttgtatagaaaagttgctttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgcgcaagtttgtacaaaaaagcaggctttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccctcatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagatacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaacttta

SEQ ID NO: 16 - DNA, 12537 bp, Artificial Sequence
  Binary vector pMPB0086 comprising WP07::gfp-nos expression cassette wherein WP07 promoter is 2400bp in length and 35S::hph-35S selection cassette for use as Entry Clone with Gateway Destination Clone.
ttatacatagttgataattcactggccgtcgttttacaactcgacctgcaggcatgcaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcgagctcggtacccggggatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattaggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatccttatagagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtcttcatgatgaagtgacagatagctgggcaatggaatccgaggaggtttcccgaaattaccctttgttggaaagtctcaattgccctttggtcttctgagactgtatccttgatatttttggagtagaccagagtgtcgtgctccaccatgttgacgaagattttcttcttgtcattgagtcgtaagagactctgtatgaactgttcgccagttttcacggcgagttctgttagatcctcgatttgaatctttgactccatggcctttgattcagtaggaactacttttttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttaatgagacctgctgcgtaggcctctctaaccatctgtgggttagcgttctttctgaaattgaagaggctaatcttctcattatcagtggtgaacatagtatcgtcaccttcaccgtcgaactttcttcctagatcgtagagatagaggaagtcgtccattgtaatctccggggcaaaggagatcctctagagtcgagggtacccggggatcctctagagtcgagggtacccggggatcctctagagtcgaatgattacgccaagctatcaactttgtatagaaaagttgctttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgcatggttacgtatacactaaacgtaccgaatgtcagagcacgcagttgtgctgctgtctcgcactcttatagaacggcaaaatcacgctcaccttatcttggattgcttatgtacatatcctgggaaatacgtcagcttgtcaatccaaaactattttctagatggagctatgcgctttcagttagagagtaagtccatcgtgacactgcagcgaactcgttcacctctttccagcacccggctgcacacgcggccgcccggttggcgctaatcaccttctataacactttgccatcacctctgccgcattgctcgatcggctgagcgaacaccatgcaagtttgtacaaaaaagcaggctttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccctcatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagatacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaacttta

SEQ ID NO: 17 - DNA, 9312 bp, Artificial Sequence
  Binary vector pMPB0087 comprising WP07::gfp-nos expression cassette wherein WP07 promoter is 2400bp in length, and ubi::bar-nos selection cassette for use as Entry Clone with Gateway Destination Clone.
ttatacatagttgataattcactggccgtcgtgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcattttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgaattcctgcagccccgccaagctatcaactttgtatagaaaagttgctttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgcatggttacgtatacactaaacgtaccgaatgtcagagcacgcagttgtgctgctgtctcgcactcttatagaacggcaaaatcacgctcaccttatcttggattgcttatgtacatatcctgggaaatacgtcagcttgtcaatccaaaactattttctagatggagctatgcgctttcagttagagagtaagtccatcgtgacactgcagcgaactcgttcacctctttccagcacccggctgcacacgcggccgcccggttggcgctaatcaccttctataacactttgccatcacctctgccgcattgctcgatcggctgagcgaacaccatgcaagtttgtacaaaaaagcaggctttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccctcatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagatacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaacttta

SEQ ID NO: 18 - DNA, 13518 bp, Artificial Sequence
  Binary vector RHF112qc comprising WP05::GUS-nos expression cassette.
gtgattttgtgccgagctgccggtcggggagctgttggctggctggtggcaggatatattgtggtgtaaacaaattgacgcttagacaacttaataacacattgcggacgtctttaatgtactgaattagtactctagtttacagcactcgtctccgtcttggtaggttctttgagcttaagaaggttgacgttgtggtgataggtctaaggcggaggctaggctagttgatatcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatcctcatctaagcgcaaagagacgtactatggaaaacgctaaaatgaactcgctcatcgcccagtatccgttggtaaaggatctggttgctcttaaagaaaccacctggtttaatcctggcacgacctcattggctgaaggtttaccttatgttggcctgaccgaacaggatgttcaggacgcccatgcgcgcttatcccgttttgcaccctatctggcaaaagcatttcctgaaactgctgccactggggggattattgaatcagaactggttgccattccagctatgcaaaaacggctggaaaaagaatatcagcaaccgatcagcgggcaactgttactgaaaaaagatagccatttgcccatttccggctccataaaagcacgcggcgggatttatgaagtcctggcacacgcagaaaaactggctctggaagcggggttgctgacgcttgatgatgactacagcaaactgctttctccggagtttaaacagttctttagccaatacagcattgctgtgggctcaaccggaaatctggggttatcaatcggcattatgagcgcccgcattggctttaaggtgacagttcatatgtctgctgatgcccgggcatggaaaaaagcgaaactgcgcagccatggcgttacggtcgtggaatatgagcaagattatggtgttgccgtcgaggaaggacgtaaagcagcgcagtctgacccgaactgtttctttattgatgacgaaaattcccgcacgttgttccttgggtattccgtcgctggccagcgtcttaaagcgcaatttgcccagcaaggccgtatcgtcgatgctgataaccctctgtttgtctatctgccgtgtggtgttggcggtggtcctggtggcgtcgcattcgggcttaaactggcgtttggcgatcatgttcactgcttttttgccgaaccaacgcactccccttgtatgttgttaggcgtccatacaggattacacgatcagatttctgttcaggatattggtatcgacaaccttaccgcagcggatggccttgcagttggtcgcgcatcaggctttgtcgggcgggcaatggagcgtctgctggatggcttctatacccttagcgatcaaaccatgtatgacatgcttggctggctggcgcaggaagaaggtattcgtcttgaaccttcggcactggcgggtatggccggacctcagcgcgtgtgtgcatcagtaagttaccaacagatgcacggtttcagcgcagaacaactgcgtaataccactcatctggtgtgggcgacgggaggtggaatggtgccggaagaagagatgaatcaatatctggcaaaaggccgttaataacgtttcaacgcagcatggatcgtaccgagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgtacaacgcgtaccggttagccttaattaattgtagctaaagttaaataaaaggctgaatttattgcatgggagaagagagagattgggtctggcacattgctcttagttctgcgtgtaaaatccagtgatggggtagctcctgaaataccaatatcttataagtaaagaaaagttttttatacaatacatatgtcaatatacggtgcaaggtgtcgctagctgactcgctaggcaggtgccggtaggtatcggcaccgctcaatttttcactttaatacatactagcatcccccggattgtagcattttaccacaaaaatactagcaaagggcattactcactttttagctaacttgtgaacattcactatatagaaaattccaaatgataggtttgtcactcttgagagggcgccaaagttggatttgtctccacatataaagggctaaacagaaagccagctggtgcttatgagatagaaactgatcacagaacgcatggatcaaaccccttgcatgcgtaccttcccaaaatagtggctagcaaaccacggacacatacatccatccttgagtttatcttaggatgaaacatccatccttgagcatcacggaaaaatacacaaacagaaagcgcgagtggacggctgtatgcagcggaaagcatccaaaatgggaagctgtatcctgtatgtatgtatgcagccatctcttcccggcgtcgtttctggcccagacggctcacacacacgcccctcgcaccctgaaggctgctgtgcagtacacgttgagaggcgctctacggtccatagataagcatgcatgcgtgcatgcatggccggagaagcaagggaagaacgctgcagagacggtgagcaccaggaccaggtgtaggtgcaaccagtcttgcgtgaattagggtggcacgcatccagacctttttaagattttaattaacacgagggccgtttgtgtgcttggctcaagcgcgcgtggggacaaaagctgtttcagcaaagcaatggacagatacgtgctgctaaggctgaagcaacacgggagttcatactggcatggcatcccctgggtcctatataaaggccaccctcctcgtgtccaccttcccactcatcgcaacccacctctggcttccaagtctatacataatatagggaccgagcttgcggttttgccaaggtaacacatctagtatctgcttagcttggttcttcttacagatactcctaagattgagcattaactgatcgattcgatggtacgtgcgtgcatgcaaacacaaaacgcagggtgcagcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcaactttattatacatagttgataattcactgggccggccctgtctatcttgttgggaaaagccgacctacccggacgcgattacttaagcaaaagatactatcgaacgaagaaagctagtaggtagactatatcaggcctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 19 - DNA, 14644 bp, Artificial Sequence
  Binary vector RHF112qc comprising WP07::GUS-nos expression cassette whreint he WP07 promoter is 2400bp in length.
tcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaattagtactctagtttacagcactcgtctccgtcttggtaggttctttgagcttaagaaggttgacgttgtggtgataggtctaaggcggaggctaggctagttgatatcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatcctcatctaagcgcaaagagacgtactatggaaaacgctaaaatgaactcgctcatcgcccagtatccgttggtaaaggatctggttgctcttaaagaaaccacctggtttaatcctggcacgacctcattggctgaaggtttaccttatgttggcctgaccgaacaggatgttcaggacgcccatgcgcgcttatcccgttttgcaccctatctggcaaaagcatttcctgaaactgctgccactggggggattattgaatcagaactggttgccattccagctatgcaaaaacggctggaaaaagaatatcagcaaccgatcagcgggcaactgttactgaaaaaagatagccatttgcccatttccggctccataaaagcacgcggcgggatttatgaagtcctggcacacgcagaaaaactggctctggaagcggggttgctgacgcttgatgatgactacagcaaactgctttctccggagtttaaacagttctttagccaatacagcattgctgtgggctcaaccggaaatctggggttatcaatcggcattatgagcgcccgcattggctttaaggtgacagttcatatgtctgctgatgcccgggcatggaaaaaagcgaaactgcgcagccatggcgttacggtcgtggaatatgagcaagattatggtgttgccgtcgaggaaggacgtaaagcagcgcagtctgacccgaactgtttctttattgatgacgaaaattcccgcacgttgttccttgggtattccgtcgctggccagcgtcttaaagcgcaatttgcccagcaaggccgtatcgtcgatgctgataaccctctgtttgtctatctgccgtgtggtgttggcggtggtcctggtggcgtcgcattcgggcttaaactggcgtttggcgatcatgttcactgcttttttgccgaaccaacgcactccccttgtatgttgttaggcgtccatacaggattacacgatcagatttctgttcaggatattggtatcgacaaccttaccgcagcggatggccttgcagttggtcgcgcatcaggctttgtcgggcgggcaatggagcgtctgctggatggcttctatacccttagcgatcaaaccatgtatgacatgcttggctggctggcgcaggaagaaggtattcgtcttgaaccttcggcactggcgggtatggccggacctcagcgcgtgtgtgcatcagtaagttaccaacagatgcacggtttcagcgcagaacaactgcgtaataccactcatctggtgtgggcgacgggaggtggaatggtgccggaagaagagatgaatcaatatctggcaaaaggccgttaataacgtttcaacgcagcatggatcgtaccgagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgtacaacgcgtaccggttaattaattgttgaaggaaatatgccctagaggcaataataaagttgttatttatatttccttatatcatgataaatgtttattattcatgctagaattgtattaaccggaaacttggtacatgtgtgaatacatagacaaaacaaagtgtccctagtatgcctctacttgactagctcgttaatcaaagacggttaagtttcctgaccatagacatgtgttgtcatttgatgaacaaaatcacataattaggagaatgatgtgatggacatgacccatccgttagcttagcataatgatcgttaagttttattgctattgctttcttcatgacttatacatgttcctttgactatgagattatgcaactcccgtataccggaggaacaccttgtgtgctatgaaacgtcacaacgtaactgggtgattataaagatgctctataggtgtctctgaaggcgtttgttgggtttacatacatcgagtttaggacttgtcactccgagtatcagagaggtatctctgggccctctcggtaatgcgcatcacgataagccttgcaagcaacgtgactagtgagttagttgcgggatgatgcattacggaacgcgtaaagagacttttcagtaacgagattgaactaggtatgaagataccgatgatcgaatctcgggcaagtaacataccgatgacaaaaggaatgacgtatgttgtcattgcggtttgaccaataaagatcttcgtagaacatgtaggaaccaatatgagcatccaggtttcgctgttggttattgaccggagatgtgtctcgatcatgtctacatagttctcgaacccgtagggtccccacacttaacgttcgatgacgatttgtatcatgagttatgtgttttggtgaccgaagattgttcggagtcccgaatgagatcggggtctcgaaatggccgagatgtaaagattgatatattggacgatagtattcagacaccggaattgttttggagtgttttgggttttttcggagtaccgggaggttaccggaaccccccggggaagtaatgggccaacatgggccagaggggagaaagagggaagccctcaaggggtggcgcacccgccccccatgggcggtccgaattggacaaggggagggggcgcaacccccctttccttccccctctccctctcctttcccctttccccctccgaaaggaggaaggggtggccaacttggactaggagtcctagtcggtttccccccatggcaccccccttagaccagcctcctcccctcctttatatacgggggcagggggcaccccaaagcacatcaattgttcttttagccgtgtgaggtgcccccctccacagtttactcctcaggtcatattgccgtagtgcttaggcgaagccctgcgcagatcacatcaccatcaccgtcaccacgccgtcgtgctgacaaaactcttcctcgacactttgctggatcaagagttcgagggacaacatcgagctgaacgtgtgcagaactcggaggtgtcgtacgttcggtgcttgatcggttggatcgcgaagacgttcgactacatcaaccgtgtcaagctaatgcttccgcttttggcctacgggggtatgtggacacactctccccctctcgttgatatgcatcttctagatagatcttgtgtattcgtatggaaacaactcctcccattaaacgaacattgctgaaaagtctgcaataaattcagaaaaatacgagcccgattgtcacgcctagaacttcccgggtgggctggttccaccacaagaaatctaatcatctaagctacactcagtttgcaatccacctcacatattcaacaattagagtagagtgaaatttccgtggcttaaaactgacatcgagcatcgttcccaatgctcagatttgtagcagggaaaatgtgacgacacacatggggtcgtctcgcactttttgtttccatttttcttttgcgcgtaggaaaaaagccagcatagttgttgattgtttttgaaatagccaaccgctcgaacagtagtcaacataattgcatggttacgtatacactaaacgtaccgaatgtcagagcacgcagttgtgctgctgtctcgcactcttatagaacggcaaaatcacgctcaccttatcttggattgcttatgtacatatcctgggaaatacgtcagcttgtcaatccaaaactattttctagatggagctatgcgctttcagttagagagtaagtccatcgtgacactgcagcgaactcgttcacctctttccagcacccggctgcacacgcggccgcccggttggcgctaatcaccttctataacactttgccatcacctctgccgcattgctcgatcggctgagcgaacaccatccggcgccctaggcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcaactttattatacatagttgataattcactgggccggccctgtctatcttgttgggaaaagccgacctacccggacgcgattacttaagcaaaagatactatcgaacgaagaaagctagtaggtagactatatcaggcctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttac